(19) United States
(12) Patent Application Publication
(10) Pub. No.: US 2025/0179181 A1
(43) Pub. Date: Jun. 5, 2025

(54) MULTI-SPECIFIC BINDING PROTEINS AND METHODS OF USE THEREOF

(71) Applicant: Cullinan Therapeutics, Inc., Cambridge, MA (US)

(72) Inventors: Patrick A. Baeuerle, Cambridge, MA (US); Jennifer Michaelson, Brighton, MA (US); Bochong Li, Boston, MA (US); Naveen Mehta, Cambridge, MA (US)

(21) Appl. No.: 19/044,246

(22) Filed: Feb. 3, 2025

Related U.S. Application Data
(63) Continuation of application No. 16/972,460, filed on Aug. 11, 2023.
(60) Provisional application No. 62/681,784, filed on Jun. 7, 2018.

(51) Int. Cl. C07K 16/28; A61K 39/00; A61P 35/00; C07K 16/18

(52) U.S. Cl. CPC C07K 16/2809; A61P 35/00; C07K 16/18; C07K 16/2803; A61K 2039/505; C07K 2317/31; C07K 2317/565; C07K 2317/569; C07K 2317/622; C07K 2317/73; C07K 2317/76; C07K 2317/92

(57) ABSTRACT

The invention relates to multi-specific binding proteins that bind CD19, CD3, and serum albumin. The invention also relates to pharmaceutical compositions comprising these multi-specific binding proteins, expression vectors and host cells for making these multi-specific binding proteins, and methods of use of these multi-specific binding proteins in treating hematologic cancers.

### CROSS-REFERENCE TO RELATED APPLICATIONS

This application is a Continuation of U.S. patent application Ser. No. 16/972,460 filed Aug. 11, 2023, which claims priority to International Application No. PCT/US2019/036177 filed Jun. 7, 2019 and U.S. Provisional Patent Application Ser. No. 62/681,784, filed Jun. 7, 2018, each of which is hereby incorporated by reference in its entirety.

### INCORPORATION BY REFERENCE OF SEQUENCE LISTING

This application contains a Sequence listing that has been submitted in a computer readable format and is hereby incorporated by reference in its entirety. The xml file, created on Jan. 8, 2025, is named 67254US02_SequenceListing.xml, and is 557,584 bytes in size.

### FIELD OF THE INVENTION

The invention relates to multi-specific binding proteins that bind CD19, CD3, and serum albumin. The invention also relates to pharmaceutical compositions comprising these multi-specific binding proteins, expression vectors and host cells for making these multi-specific binding proteins, and methods of use of these multi-specific binding proteins in treating hematologic cancers.

### BACKGROUND

Bispecific molecules available under the trade name BiTE® from Micromet AG (bispecific T-cell engager) constructs are recombinant protein constructs made from two flexibly linked antibody-derived binding domains. One binding domain of BiTE® constructs is specific for a selected tumor-associated surface antigen on target cells, and the second binding domain is specific for CD3, a subunit of the T cell receptor complex on T cells. By this design, BiTE® constructs can transiently connect T cells with target cells and, at the same time, potently activate the inherent cytolytic potential of T cells against target cells.

The CD3 receptor complex is a protein complex composed of four polypeptide chains. In mammals, the complex contains a CD3γ (gamma) chain, a CD3δ (delta) chain, and two CD3ε (epsilon) chains. The CD3γ (gamma), CD3δ (delta), and CD3ε (epsilon) chains are highly related cell-surface proteins of the immunoglobulin superfamily containing a single extracellular immunoglobulin domain. These chains associate with the T cell receptor (TCR) to form a TCR-CD3 complex and to generate an activation signal in T lymphocytes upon antigen engagement. About 95% of T cells express αβ TCR, which contains an a (alpha) chain and a R (beta) chain. Two TCR4 (zeta) chains are also present in the TCR-CD3 complex. The αβ TCR is responsible for recognizing antigens presented by a major histocompatibility complex (MHC). When the TCR engages with antigenic peptide and MHC complex, the T lymphocyte is activated through a series of biochemical events mediated by associated enzymes, co-receptors, specialized adaptor molecules, and activated or released transcription factors.

Earlier BiTE® constructs bind conformational epitopes of CD3 and typically are species-specific (see, PCT Publication No. WO2008119567A2). Improved BiTE® constructs, such as blinatumomab (also called AMG 103; see, PCT Publication No. WO1999054440A1) and solitomab (also called AMG 110; see, PCT Publication No. WO2005040220A1), bind context-independent epitopes at the N-terminus of CD3ε chain (e.g., amino acid residues 1-27 of human CD3ε extracellular domain) and show cross-species specificity for human, Callithrix jacchus, Saguinus Oedipus, and Saimiri sciureus CD3ε chain (see id.). These constructs do not nonspecifically activate T cells to the same degree as observed with the earlier BiTE® constructs, and are therefore believed to bear a lower risk of side effects (see, Brischwein et al. (2007) J. Immunother., 30(8): 798-807).

BiTE® constructs are believed to suffer from rapid clearance from the body. Therefore, whilst they are able to rapidly penetrate many areas of the body, and are quick to produce and easier to handle, their in vivo applications may be limited by their brief persistence in vivo. Prolonged administration by continuous intravenous infusions may be required to achieve therapeutic effects of blinatumomab and solitomab because of their short in vivo half-life. However, such continuous intravenous infusions are inconvenient for patients and may increase the costs of treatment.

Although significant developments have been made in constructing multi-specific binding proteins, there remains a need for new and useful multi-specific binding proteins for treating cancer that have adequate therapeutic efficacy, a format straightforward to manufacture, and favorable pharmacokinetic properties such as a longer half-life.

### SUMMARY OF THE INVENTION

The multi-specific binding proteins disclosed herein comprise a first domain that binds CD19 (e.g., human CD19), a second domain that binds CD3 (e.g., human and/or Macaca CD3), and a third domain that binds serum albumin (e.g., human serum albumin (HSA)). These domains are linked in certain manners for favorable therapeutic efficacy and in vivo half-life. The multi-specific binding proteins can be used to stimulate an immune response against a cell expressing CD19. As a result, the multi-specific binding proteins can be used to treat a disease or disorder associated with aberrant cells expressing CD19, such as certain hematologic B-cell cancers.

In one aspect, the present disclosure provides a multi-specific binding protein comprising: (a) a first antigen-binding site that binds human CD19; (b) a second antigen-binding site that binds human CD3; and (c) a third antigen-binding site that binds human serum albumin (HSA).

In certain embodiments, the multi-specific binding protein comprises a single polypeptide chain. In certain embodiments, the third antigen-binding site is not positioned between the first antigen-binding site and the second antigen-binding site in the polypeptide chain.

In certain embodiments, the third antigen-binding site is positioned N-terminal to both the first antigen-binding site and the second antigen-binding site in the polypeptide chain. In certain embodiments, the third antigen-binding site is positioned N-terminal to the first antigen-binding site, and the first antigen-binding site is positioned N-terminal to the second antigen-binding site in the polypeptide chain. In certain embodiments, the third antigen-binding site is positioned N-terminal to the second antigen-binding site, and the second antigen-binding site is positioned N-terminal to the first antigen-binding site in the polypeptide chain.

In certain embodiments, the third antigen-binding site is positioned C-terminal to both the first antigen-binding site and the second antigen-binding site in the polypeptide chain. In certain embodiments, the first antigen-binding site is positioned N-terminal to the second antigen-binding site, and the second antigen-binding site is positioned N-terminal to the third antigen-binding site in the polypeptide chain. In certain embodiments, the second antigen-binding site is positioned N-terminal to the first antigen-binding site, and the first antigen-binding site is positioned N-terminal of the third antigen-binding site in the polypeptide chain.

In certain embodiments, the first antigen-binding site is positioned N-terminal to the third antigen-binding site, and the third antigen-binding site is positioned N-terminal to the second antigen-binding site in the polypeptide chain. In certain embodiments, the second antigen-binding site is positioned N-terminal to the third antigen-binding site, and the third antigen-binding site is positioned N-terminal binding protein the first antigen-binding site in the polypeptide chain.

In certain embodiments, the first antigen-binding site comprises a single-chain variable fragment (scFv) or a single-domain antibody (sdAb). In certain embodiments, the first antigen-binding site comprises a single-chain variable fragment (scFv). In certain embodiments, the first antigen-binding site binds human CD19 with a dissociation constant (K_{D}) equal to or lower than 20 nM (namely, binding equal to or stronger than 20 nM). In certain embodiments, the first antigen-binding site has a melting temperature of at least 60° C.

In certain embodiments, the first antigen-binding site comprises a heavy chain variable domain (VH) comprising complementarity determining regions HCDR1, HCDR2, and HCDR3, and a light chain variable domain (VL) comprising complementarity determining regions LCDR1, LCDR2, and LCDR3, wherein (i) the HCDR1, HCDR2, HCDR3, LCDR1, LCDR2, and LCDR3 comprise the amino acid sequences set forth in SEQ ID NOs: 3, 4, 5, 6, 7, and 8, respectively; and/or (ii) the VH comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 1, and the VL comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 2. In certain embodiments, the VH comprises the amino acid sequence set forth in SEQ ID NO: 1, and the VL comprises the amino acid sequence set forth in SEQ ID NO: 2.

In certain embodiments, the first antigen-binding site comprises a VH comprising complementarity determining regions HCDR1, HCDR2, and HCDR3, and a VL comprising complementarity determining regions LCDR1, LCDR2, and LCDR3, wherein (i) the HCDR1, HCDR2, HCDR3, LCDR1, LCDR2, and LCDR3 comprise the amino acid sequences set forth in SEQ ID NOs: 12, 13, 14, 15, 16, and 17, respectively; and/or (ii) the VH comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 10, and the VL comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 11. In certain embodiments, the VH comprises the amino acid sequence set forth in SEQ ID NO: 10, and the VL comprises the amino acid sequence set forth in SEQ ID NO: 11.

In certain embodiments, the second antigen-binding site comprises an scFv or an sdAb. In certain embodiments, the second antigen-binding site comprises an scFv. In certain embodiments, the second antigen-binding site binds human CD3ε. In certain embodiments, the second antigen-binding site binds human CD3 with a K_{D} equal to or lower than 10 nM (namely, binding equal to or stronger than 10 nM). In certain embodiments, the second antigen-binding site binds human CD3ε with a K_{D} equal to or lower than 10 nM. In certain embodiments, the second antigen-binding site has a melting temperature of at least 60° C.

In certain embodiments, the second antigen-binding site comprises a VH comprising complementarity determining regions HCDR1, HCDR2, and HCDR3, and a VL comprising complementarity determining regions LCDR1, LCDR2, and LCDR3, wherein (i) the HCDR1, HCDR2, HCDR3, LCDR1, LCDR2, and LCDR3 comprise the amino acid sequences set forth in SEQ ID NOs: 99, 100, 101, 102, 103, and 104, respectively; and/or (ii) the VH comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 97, and the VL comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 98. In certain embodiments, the VH comprises the amino acid sequence set forth in SEQ ID NO: 97, and the VL comprises the amino acid sequence set forth in SEQ ID NO: 98.

In certain embodiments, the third antigen-binding site comprises an scFv or an sdAb. In certain embodiments, the third antigen-binding site comprises an sdAb. In certain embodiments, the third antigen-binding site binds HSA with a K_{D} equal to or lower than 20 nM (namely, binding equal to or stronger than 20 nM). In certain embodiments, the third antigen-binding site has a melting temperature of at least 60° C.

In certain embodiments, the third antigen-binding site comprises a VH comprising complementarity determining regions HCDR1, HCDR2, and HCDR3, wherein (i) the HCDR1, HCDR2, and HCDR3 comprise the amino acid sequences set forth in SEQ ID NOs: 122 or 123, 124 or 125, and 126, respectively; and/or (ii) the VH comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 121. In certain embodiments, the VH comprises the amino acid sequence set forth in SEQ ID NO: 121.

In certain embodiments, at least two adjacent antigen-binding sites are connected by a peptide linker. In certain embodiments, each of the adjacent antigen-binding sites in the multi-specific binding protein are connected by a peptide linker. In certain embodiments, the peptide linker comprises the amino acid sequence of SEQ ID NO: 298, 299, or 302. In certain embodiments, the peptide linker consists of the amino acid sequence of SEQ ID NO: 298, 299, or 302.

In certain embodiments, the multi-specific binding protein does not comprise an antibody Fc region.

In certain embodiments, the molecular weight of the multi-specific binding protein is at least 65 kD. In certain embodiments, the molecular weight of the multi-specific binding protein is in the range of 50-90 kD, 50-80 kD, 50-70 kD, 50-60 kD, 60-90 kD, 60-80 kD, 60-70 kD, 65-90 kD, 65-80 kD, 65-70 kD, 70-90 kD, or 70-80 kD.

In certain embodiments, the serum half-life of the multi-specific binding protein is at least 24, 36, 48, or 60 hours.

In another aspect, the present disclosure provides a pharmaceutical composition comprising a multi-specific binding protein disclosed herein and a pharmaceutically acceptable carrier.

In another aspect, the present disclosure provides an isolated polynucleotide encoding a multi-specific binding protein disclosed herein. In another aspect, the present disclosure provides a vector comprising the polynucleotide. In another aspect, the present disclosure provides a recombinant host cell comprising the polynucleotide or the vector.

In another aspect, the present disclosure provides a method of producing a multi-specific binding protein, the method comprising culturing a host cell disclosed herein under suitable conditions that allow expression of the multi-specific binding protein. In certain embodiments, the method further comprises isolating the multi-specific binding protein. In certain embodiments, the method further comprises formulating the isolated multi-specific binding protein with a pharmaceutically acceptable carrier.

In another aspect, the present disclosure provides a method of stimulating an immune response against a cell expressing CD19, the method comprising exposing the cell and a T lymphocyte to a multi-specific binding protein or pharmaceutical composition disclosed herein.

In another aspect, the present disclosure provides a method of treating a hematologic cancer in a subject in need thereof, the method comprising administering to the subject an effective amount of a multi-specific binding protein or pharmaceutical composition disclosed herein. In certain embodiments, the hematologic cancer is a B-cell hematologic malignancy.

In another aspect, the present disclosure provides a complex comprising a T cell expressing CD3, a B cell expressing CD19, and a multi-specific binding protein disclosed herein, wherein the T cell and the B cell are simultaneously bound by the multi-specific binding protein. In certain embodiments, the complex further comprises HSA.

### BRIEF DESCRIPTION OF THE DRAWINGS

FIG. 1 is a schematic representation of six domain arrangements of single-chain multi-specific binding proteins. The CD19 binding domain in the form of a single-domain antibody (sdAb), the CD3 binding domain in the form of a single-chain variable fragment (scFv), and the HSA binding domain in the form of an sdAb are linked in different orientations. The top of each construct represents the N-terminus and the bottom of each construct represents the C-terminus of a given polypeptide chain.

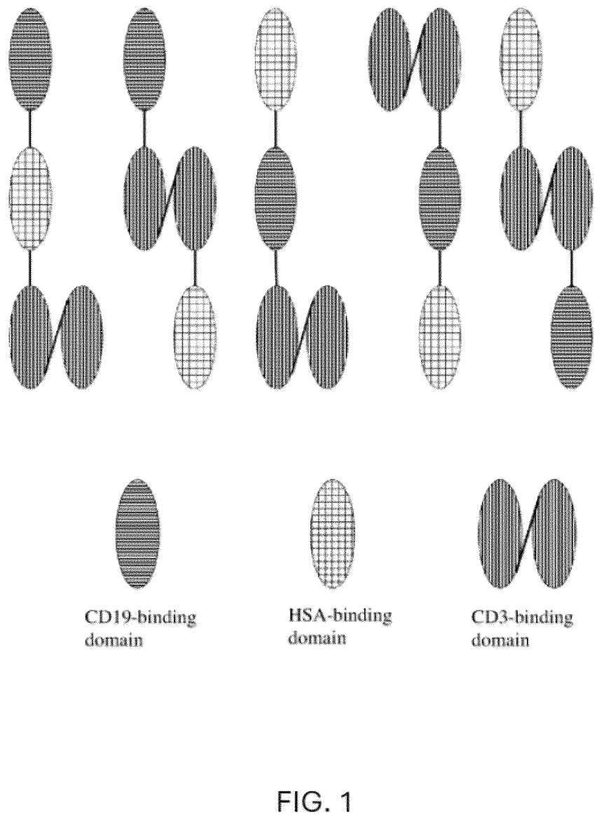

FIG. 2 is a schematic representation of six domain arrangements of single-chain multi-specific binding proteins. The CD19 binding domain in the form of an scFv, the CD3 binding domain in the form of an scFv, and the HSA binding domain in the form of an sdAb are linked in different orientations. The top of each construct represents the N-terminus and the bottom of each construct represents the C-terminus of a given polypeptide chain.

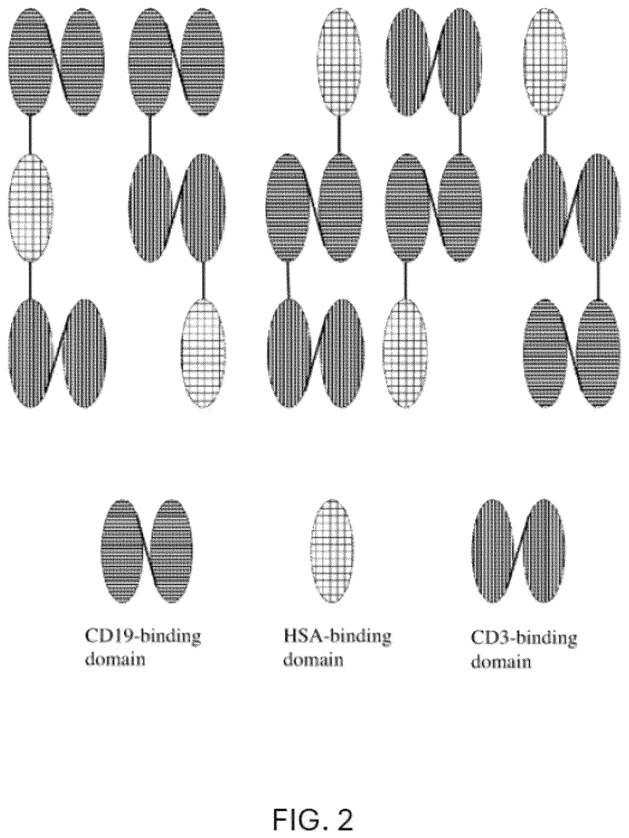

### DETAILED DESCRIPTION

The multi-specific binding proteins disclosed herein comprise a first domain that binds CD19 (e.g., human CD19), a second domain that binds CD3 (e.g., human CD3), and a third domain that binds serum albumin (e.g., HSA). These domains are linked in certain manners for favorable therapeutic efficacy and in vivo half-life. Also provided are pharmaceutical compositions comprising the multi-specific binding proteins, methods of treating a disease or disorder using the multi-specific binding proteins or pharmaceutical compositions, and methods of producing the multi-specific binding proteins. Various aspects of the invention are set forth below in sections; however, aspects of the invention described in one particular section are not to be limited to any particular section.

To facilitate an understanding of the present invention, a number of terms and phrases are defined below.

The term “multi-specific binding protein” refers to a multi-specific molecule in which the structure and/or function is/are based on the structure and/or function of an antibody, e.g., a full-length or whole immunoglobulin molecule, or based on the heavy chain variable domain (VH) and/or light chain variable domain (VL) of an antibody, and/or single chain variants thereof. A multi-specific binding protein is hence capable of binding to its specific target or antigen. Furthermore, any one of the binding domains of a multi-specific binding protein according to the invention comprises the minimum structural requirements of an antibody which allow for the target binding. This minimum requirement may be, e.g., defined by the presence of at least the three heavy chain CDRs (i.e. CDR1, CDR2 and CDR3 of the VH domain) and/or the three light chain CDRs (i.e. CDR1, CDR2 and CDR3 of the VL domain). An alternative approach to defining the minimal structural requirements of an antibody is defining the epitope of a specific target to which the antibody binds, or by referring to a known antibody with which the antibody competes to bind to the same epitope that the known antibody binds. The antibodies on which the constructs according to the invention are based include for example monoclonal, recombinant, chimeric, deimmunized, humanized and human antibodies.

Any one of the binding domains of a multi-specific binding protein according to the invention may comprise the above referred groups of CDRs. Those CDRs may be comprised in the framework of a VH and/or VL. Fd fragments, for example, have two VH domains and often retain some antigen-binding function of the intact antigen-binding domain. Additional examples for formats of antibody fragments, antibody variants or binding domains include: (1) a Fab fragment, a monovalent fragment having the VL, VH, CL and CH1 domains; (2) a F(ab′)₂ fragment, a bivalent fragment having two Fab fragments linked by a disulfide bridge at the hinge region; (3) an Fd fragment having the two VH and CH1 domains; (4) an Fv fragment having the VL and VH domains of a single arm of an antibody; (5) a dAb fragment (Ward et al., (1989) Nature 341:544-546), which has a VH domain; (6) an isolated complementarity determining region (CDR); and (7) a single chain Fv (scFv), which may be derived, for example, from an scFv-library. Exemplary formats of multi-specific binding proteins according to the invention are described in, e.g., WO2000006605A2, WO2005040220A1, WO2008119567A2, WO2010037838A2, WO2013026837A1, WO 2013026833A1, US 20140308285A1, US20140302037A1, WO2014144722A2, WO2014151910A1, and WO2015048272A1.

Multi-specific binding proteins according to the invention may also comprise modified fragments of antibodies, also called antibody variants, such as di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv, “multibodies” such as triabodies or tetrabodies, or single-domain antibodies such as nanobodies or single variable domain antibodies comprising a single variable domain, which might be VH (also called VHH in the context of an sdAb) or VL, that specifically bind an antigen or epitope independently of other V regions or domains.

As used herein, the terms “single-chain Fv,” “single-chain antibody,” and “scFv” refer to a single-polypeptide-chain antibody fragment that comprise the variable regions from both the heavy and light chains, but lack the constant regions. Generally, a single-chain antibody further comprises a peptide linker connecting the VH and VL domains which enables it to form the desired structure to bind to antigen. Single chain antibodies are discussed in detail by Pluckthun in The Pharmacology of Monoclonal Antibodies, vol. 113, Rosenburg and Moore eds. Springer-Verlag, New York, pp. 269-315 (1994). Various methods of generating single chain antibodies are known, including those described in U.S. Pat. Nos. 4,694,778 and 5,260,203; International Patent Application Publication No. WO 88/01649; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041. In specific embodiments, single-chain antibodies can also be bispecific, multispecific, human, humanized and/or synthetic.

Furthermore, the “multi-specific binding protein” described herein can be a monovalent, bivalent or polyvalent/multivalent construct. Moreover, the “multi-specific binding protein” described herein can include a molecule consisting of only one polypeptide chain, or a molecules consisting of more than one polypeptide chain, wherein the chains can be either identical (homodimers, homotrimers or homo oligomers) or different (heterodimer, heterotrimer or heterooligomer). Examples for the above identified antibodies and the variants or derivatives thereof are described, for example, in Harlow and Lane, Antibodies a laboratory manual, CSHL Press (1988); Using Antibodies: a laboratory manual, CSHL Press (1999); Kontermann and Dibel, Antibody Engineering, Springer, 2nd ed. 2010; and Little, Recombinant Antibodies for Immunotherapy, Cambridge University Press 2009.

The domains of the multi-specific binding protein of the present invention may be connected through one or more peptide bonds and/or peptide linkers. The term “peptide linker” comprises in accordance with the present invention an amino acid sequence linking two domains. The peptide linkers can also be used to fuse the third domain to the other domains of the multi-specific binding protein of the invention. An essential technical feature of such peptide linker is that it does not comprise any polymerization activity. Among the suitable peptide linkers are those described in U.S. Pat. Nos. 4,751,180 and 4,935,233 or WO198809344A1.

The multi-specific binding proteins of the present invention may be in vitro generated multi-specific binding proteins. The term “in vitro generated multi-specific binding protein” refers to a multi-specific binding protein according to the above definition where all or part of the variable region (e.g., at least one CDR) is generated by non-immune cell selection, e.g., an in vitro phage display, protein chip or any other method in which candidate sequences can be tested for their ability to bind to an antigen. The multi-specific binding proteins of the present invention may also be generated by genomic rearrangement in an immune cell in an animal. A “recombinant antibody” is an antibody made through the use of recombinant DNA technology or genetic engineering.

The multi-specific binding protein of the invention may be monoclonal. The term “monoclonal,” as used herein, means that the proteins obtained from a population are substantially homogeneous, i.e., the individual proteins in the population are identical except for naturally occurring mutations and/or post-translation modifications (e.g., isomerizations, amidations) that may be present. In the context of antibodies, monoclonal antibodies are highly specific, being directed against a single antigenic side or determinant on the antigen, in contrast to conventional (polyclonal) antibody preparations which typically include different antibodies directed against different determinants (or epitopes). The modifier “monoclonal” indicates the character of the antibody as being obtained from a substantially homogeneous population of antibodies, and is not to be construed as requiring production of the antibody by any particular method.

The multi-specific binding protein of the invention or one or more antigen-binding site thereof may be affinity matured. In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. Two or three rounds of mutation and selection using display methods such as phage display can result in antibody fragments with affinities in the low nanomolar range.

An amino acid substitution variation can be introduced into the multi-specific binding proteins by substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sides (e.g., 6-7 sides) are mutated to generate all possible amino acid substitutions at each side. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sides for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domains. Such contact residues and neighboring residues are candidates for substitution according to the techniques elaborated herein. Once such variants are generated, the panel of variants is subjected to screening as described herein and antibodies with superior properties in one or more relevant assays may be selected for further development.

The multi-specific binding proteins of the present invention specifically can comprise “chimeric” antibodies (immunoglobulins) or fragments thereof in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, so long as they exhibit the desired biological activity (U.S. Pat. No. 4,816,567; Morrison et al. (1984) Proc. Natl. Acad. Sci. U.S.A., 81: 6851-55). Chimeric antibodies of interest herein include “primatized” antibodies comprising variable domain antigen-binding sequences derived from a non-human primate (e.g., Old World Monkey, Ape etc.) or human constant region sequences. A variety of approaches for making chimeric antibodies have been described. See e.g., Morrison et al. (1985) Proc. Natl. Acad. Sci. U.S.A., 81:6851; Takeda et al. (1985) Nature, 314: 452; U.S. Pat. Nos. 4,816,567; 4,816,397; European Patent No. EP0171496; European Patent Application Publication No. EP0173494; and U.K. Patent No. GB2177096.

The term “binding domain” or “domain that binds (an antigen)” characterizes in connection with the present invention a domain which (specifically) binds to or interacts with a given target epitope or a given target side on the target molecules (antigens), e.g. CD19, serum albumin, and CD3, respectively. The structure and function of the first binding domain, the second binding domain, and/or the third binding domain can be based on the structure and/or function of an antibody, e.g. of a full-length or whole immunoglobulin molecule. A binding domain can be drawn from the VH and/or VL or VHH domain of an antibody or fragment thereof. For example, a binding domain can include three light chain CDRs (i.e., CDR1, CDR2 and CDR3 of the VL domain) and/or three heavy chain CDRs (i.e., CDR1, CDR2 and CDR3 of the VH domain). A binding domain can also include VHH CDRs (i.e., CDR1, CDR2 and CDR3 of the VHH region).

The terms “variable domain” and “variable region” are used interchangeably and refer to the portions of the antibody or immunoglobulin domains that exhibit variability in their sequence and that are involved in determining the specificity and binding affinity of a particular antibody. Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three dimensional space to form an antigen-binding surface.

In the present invention, any one of the binding domains of the multi-specific binding protein may comprise a single domain antibody (sdAb). A single domain antibody comprises a single, monomeric antibody variable domain which is able to bind selectively to a specific antigen, independently of other variable regions or domains. The first single domain antibodies were engineered from heavy chain antibodies found in camelids, and these are called VHH fragments. Cartilaginous fishes also have heavy chain antibodies (IgNAR) from which single domain antibodies called V_{NAR} fragments can be obtained. An alternative approach is to split the dimeric variable domains from common immunoglobulins e.g., from humans or rodents into monomers, hence obtaining VH or VL as a single domain antibody. Although most research into single domain antibodies is currently based on heavy chain variable domains, nanobodies derived from light chains have also been shown to bind specifically to target epitopes. Examples of single domain antibodies include nanobodies and single variable domain antibodies.

As used herein, the term “antigen-binding site” refers to the part of an immunoglobulin molecule or a derivative or variant thereof that participates in antigen binding. In human antibodies, the antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide.

The terms “a” and “an” as used herein mean “one or more” and include the plural unless the context is inappropriate.

As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably include humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA (1975).

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Multi-Specific Binding Proteins

In one aspect, the present disclosure provides a multi-specific binding protein that comprises a first domain that binds CD19 (e.g., human CD19); a second domain that binds CD3 (e.g., human and/or Macaca CD3), such as CD3ε (epsilon), CD3δ (delta), and/or CD3γ (gamma); and a third domain that binds serum albumin (e.g., HSA).

In certain embodiments, the first domain is a first antigen-binding site that binds CD19. In certain embodiments, the first antigen-binding site comprises an antibody heavy chain variable domain (VH). In certain embodiments, the first antigen-binding site comprises an antibody light chain variable domain (VL). In certain embodiments, the first antigen-binding site comprises a VH and a VL. In certain embodiments, the first antigen-binding site comprises an sdAb. In certain embodiments, the first antigen-binding site comprises an scFv.

In certain embodiments, the second domain is a second antigen-binding site that binds CD3. In certain embodiments, the second antigen-binding site comprises a VH. In certain embodiments, the second antigen-binding site comprises a VL. In certain embodiments, the second antigen-binding site comprises a VH and a VL. In certain embodiments, the second antigen-binding site comprises an sdAb. In certain embodiments, the second antigen-binding site comprises an scFv.

In certain embodiments, the third domain is a third antigen-binding site that binds serum albumin. In certain embodiments, the third antigen-binding site comprises a VH. In certain embodiments, the third antigen-binding site comprises a VL region. In certain embodiments, the third antigen-binding site comprises a VH region and a VL region. In certain embodiments, the third antigen-binding site comprises an sdAb. In certain embodiments, the third antigen-binding site comprises an scFv.

Alternatively, it is also contemplated that one or more of the binding domains may not comprise an antigen-binding site. For example, U.S. Patent Application Publication No. US20130316952A1 discloses a polypeptide that binds serum albumin having the amino acid sequence of LKEAKEKAIEELKKAGITSDYYFDLINKAKTVEGVNALKDEILKA (SEQ ID NO: 282). Additional exemplary polypeptides that bind HSA are described in Dennis et al. (2002) J. Biol. Chem., 277: 35035-43; Jacobs et al. (2015) Protein Eng. Des. Sel., 28: 385-93; and Zorzi et al. (2017) Nat. Commun., 8: 16092.

In certain embodiments, the multi-specific binding protein further comprises an antibody Fc region. The presence of an Fc region may increase the serum half-life of the multi-specific binding protein. Depending on the specific Fc subtype and variant used, the Fc region may also alter the activity (e.g., cytotoxic activity) of the multi-specific binding protein.

In other embodiments, the multi-specific binding protein does not comprise an antibody Fc region. The absence of Fc contributes to a smaller size of the multi-specific binding protein, which can exhibit improved tissue penetration and pharmacokinetic properties. In certain embodiments, the multi-specific binding proteins consists of or consists essentially of the first, second, and third antigen-binding sites and the linkers between them. In certain embodiments, the multi-specific binding proteins consists essentially of the first, second, and third antigen-binding sites.

In certain embodiments, the multi-specific binding protein binds CD19, CD3, and/or serum albumin monovalently. The exclusion of additional binding domains reduces the risk of non-specific immune cell activation and decreases the size of the multi-specific binding protein.

### A. First Antigen-Binding Site

The first antigen-binding site of the multi-specific binding protein binds CD19 (e.g., human CD19).

CD19, also known as B-cell surface antigen B4 or Leu-12, is a transmembrane protein expressed on B lymphocytes and follicular dendritic cells. CD19 is a co-receptor for the B-cell antigen receptor complex on B lymphocytes (see, Carter et al. (2002) Science, 256: 105-07; van Zelm et al. (2006) N. Eng. J. Med., 354: 1901-12). It associates with CD21, CD81, and Leu-13 and potentiates B cell receptor (BCR) signaling. Together with the BCR, CD19 modulates intrinsic and antigen receptor-induced signaling thresholds critical for clonal expansion of B cells and humoral immunity. Upon activation, the cytoplasmic tail of CD19 becomes phosphorylated, which leads to binding by Src-family kinases and recruitment of PI-3 kinase.

CD19 is a human B-cell surface marker that is expressed from early stages of pre-B cell development through terminal differentiation into plasma cells. It is also expressed on many non-Hodgkin lymphoma (NHL) cells and certain leukemias. Antibodies that bind CD19 have been developed and tested in clinical studies against cancers of lymphoid origin such as B-cell malignancies (see, e.g., Hekman et al. (1991) Cancer Immunol. Immunother., 32: 364-72; Vlasfeld et al. (1995) Cancer Immunol. Immunother., 40: 37-47; Corny et al. (1995) J. Immunother. Emphasis Tumor Immunol., 18: 231-41; and Manzke et al. (2001) Int. J. Cancer, 91: 516-22). Furthermore, a BiTE® construct called blinatumomab has been developed for clinical use.

The first antigen-binding site that binds CD19 can be derived from, for example, MT-103 (a single-chain bispecific CD19/CD3 antibody; see, Hoffman et al. (2005) Int. J. Cancer, 115: 98-104; Schlereth et al. (2006) Cancer Immunol. Immunother. 55: 503-14), a CD19/CD16 diabody (see, Schlenzka et al. (2004) Anti-cancer Drugs 15: 915-19; Kipriyanov et al. (2002) J. Immunol. 169: 137-44), BU12-saporin (see, Flavell et al. (1995) Br. J. Cancer 72: 1373-79), and anti-CD19-idarubicin (see, Rowland et al. (1993) Cancer Immunol. Immunother. 55: 503-14). Additional exemplary antigen-binding sites that bind CD19, from which the instant first antigen-binding site may be derived, are disclosed in U.S. Patent Application Publication Nos. US20170174786A1, US20090042291A1, US20160046730A1, US20070154473A1, US20090142349A1, US20180142018A1, US20090136526A1, US20060257398A1, and US20180230225A1, and PCT Publication No. WO2019057100A1.

A first antigen-binding site that binds CD19 can include a VH comprising three complementarity regions (HCDR1, HCDR2, and HCDR3) and/or a VL comprising three complementarity regions (LCDR1, LCDR2, and LCDR3). Table 1 summarizes, for each variable region, the CDRs of the variable region and scFv constructs based on the given heavy and light chain variable regions. The first antigen-binding site can be derived from the exemplary variable domain and CDR sequences as listed in Table 1.

TABLE 1
Sequences of Exemplary First Antigen-Binding Sites

| Antigen- | | |
|---|---|---|
| Binding | | |
| Site | VH and HCDRs | VL and LCDRs |
| Roche | QVQLVQSGAEVKKPGASVKVSCKASGY | DIVMTQTPLSLSVTPGQPASISCKSS |
| mAb | TFTDYIMHWVRQAPGQGLEWMGYINPY | QSLETSTGTTYLNWYLQKPGQSPQ |
| | NDGSKYTEKFQGRVTMTSDTSISTAYME | LLIYRVSKRFSGVPDRFSGSGSGTDF |
| | LSRLRSDDTAVYYCARGTYYYGPQLFD | TLKISRVEAEDVGVYYCLQLLEDPY |
| | YWGQGTTVTVSS (SEQ ID NO: 1) | TFGQGTKLEIK (SEQ ID NO: 2) |
| | HCDR1: DYIMH (SEQ ID NO: 3) | LCDR1: KSSQSLETSTGTTYLN (SEQ |
| | HCDR2: YINPYNDGSKYTEKFOG (SEQ | ID NO: 6) |
| | ID NO: 4) | LCDR2: RVSKRFS (SEQ ID NO: 7) |
| | HCDR3: GTYYYGPQLEDY (SEQ ID NO: 5) | LCDR3: LQLLEDPYT (SEQ ID NO: 8) |
| | scFv: | |
| | QVQLVQSGAEVKKPGASVKVSCKASGYTFTDYIMHWVRQAPGQGLEWMGYIN | |
| | PYNDGSKYTEKFQGRVTMTSDTSISTAYMELSRLRSDDTAVYYCARGTYYYGPQ | |
| | LFDYWGQGTTVTVSSGGGGSGGGGSGGGGSDIVMTQTPLSLSVTPGQPASISCKS | |
| | SQSLETSTGTTYLNWYLQKPGQSPQLLIYRVSKRFSGVPDRFSGSGSGTDFTLKIS | |
| | RVEAEDVGVYYCLQLLEDPYTFGQGTKLEIK (SEQ ID NO: 9) | |
| SG mAb | QVQLQESGPGLVKPSQTLSLTCTVSGGSI | EIVLTQSPATLSLSPGERATLSCSAS |
| | STSGMGVGWIRQHPGKGLEWIGHIWWD | SSVSYMHWYQQKPGQAPRLLIYDT |
| | DDKRYNPALKSRVTISVDTSKNQFSLKL | SKLASGIPARFSGSGSGTDFTLTISSL |
| | SSVTAADTAVYYCARMELWSYYFDYW | EPEDFAVYYCFQGSVYPFTFGQGTK |
| | GQGTLVTVSS (SEQ ID NO: 10) | LEIKR (SEQ ID NO: 11) |
| | HCDR1: TSGMGVG (SEQ ID NO: 12) | LCDR1: SASSSVSYMH (SEQ ID NO: |
| | HCDR2: HIWWDDDKRYNPALKS (SEQ | 15) |
| | ID NO: 13) | LCDR2: DTSKLAS (SEQ ID NO: 16) |
| | HCDR3: MELWSYYFDY (SEQ ID NO: 14) | LCDR3: FQGSVYPFT (SEQ ID NO: 17) |
| | scFv: | |
| | QVQLQESGPGLVKPSQTLSLTCTVSGGSISTSGMGVGWIRQHPGKGLEWIGHIW | |
| | WDDDKRYNPALKSRVTISVDTSKNQFSLKLSSVTAADTAVYYCARMELWSYYF | |
| | DYWGQGTLVTVSSGGGGSGGGGSGGGGSEIVLTQSPATLSLSPGERATLSCSASS | |
| | SVSYMHWYQQKPGQAPRLLIYDTSKLASGIPARFSGSGSGTDFTLTISSLEPEDFA | |
| | VYYCFQGSVYPFTFGQGTKLEIKR (SEQ ID NO: 18) | |
| Xencor | EVQLVESGGGLVKPGGSLKLSCAASGYT | DIVMTQSPATLSLSPGERATLSCRSS |
| mAb | FTSYVMHWVRQAPGKGLEWIGYINPYN | KSLQNVNGNTYLYWFQQKPGQSPQ |
| | DGTKYNEKFQGRVTISSDKSISTAYMELS | LLIYRMSNLNSGVPDRFSGSGSGTE |
| | SLRSEDTAMYYCARGTYYYGTRVFDYW | FTLTISSLEPEDFAVYYCMQHLEYPI |
| | GQGTLVTVSS (SEQ ID NO: 19) | TFGAGTKLEIK (SEQ ID NO: 20) |
| | HCDR1: SYVMH (SEQ ID NO: 21) | LCDR1: RSSKSLQNVNGNTYLY |
| | HCDR2: WIGYINPYNDGTKY (SEQ ID | (SEQ ID NO: 24) |
| | NO: 22) | LCDR2: RMSNLNS (SEQ ID NO: 25) |
| | HCDR3: GTYYYGTRVFDY (SEQ ID NO: | LCDR3: MQHLEYPIT (SEQ ID NO: |
| | 23) | 26) |
| Abbvie | QVQLQQSGAELVRPGSSVKISCKASGYA | DILLTQTPASLAVSLGQRATISCKAS |
| mAb | FSSYWMNWVKQRPGQGLEWIGQIWPG | QSVDYDGDSYLNWYQQIPGQPPKL |
| | DGDTNYNGKFKGKATLTADESSSTAYM | LIYDASNLVSGIPPRFSGSGSGTDFT |
| | QLSSLASEDSAVYFCARRETTTVGRYYY | LNIHPVEKVDAATYHCQQSTEDPW |
| | AMDYWGQGTSVTVSS (SEQ ID NO: 27) | TFGGGTKLEIK (SEQ ID NO: 28) |
| | HCDR1: SYWMN (SEQ ID NO: 29) | LCDR1: KASQSVDYDGDSYLN (SEQ |
| | HCDR2: QIWPGDGDTNYNGKFKG (SEQ | ID NO: 32) |
| | ID NO: 30) | LCDR2: DASNLVS (SEQ ID NO: 33) |
| | HCDR3: RETTTVGRYYYAMDY (SEQ ID | LCDR3: QQSTEDPWT (SEQ ID NO: |
| | NO: 31) | 34) |
| Immuno | QVQLQQSGAEVKKPGSSVKVSCKASGY | DIQLTQSPSSLSASVGDRVTITCKAS |
| medics | AFSSYWMNWVRQRPGQGLEWIGQIWPG | QSVDYDGDSYLNWYQQIPGKAPKL |
| mAb | DGDTNYNGKFKGRATITADESTNTAYM | LIYDASNLVSGIPPRFSGSGSGTDYT |
| | ELSSLRSEDTAFYSCARRETTTVGRYYY | FTISSLQPEDIATYHCQQSTEDPWTF |
| | AMDYWGQGTTVTVSS (SEQ ID NO: 35) | GGGTKLQIKR (SEQ ID NO: 36) |
| | HCDR1: SYWMN (SEQ ID NO: 29) | LCDR1: KASQSVDYDGDSYLN (SEQ |
| | HCDR2: QIWPGDGDTNYNGKFKG (SEQ | ID NO: 32) |
| | ID NO: 30) | LCDR2: DASNLVS (SEQ ID NO: 33) |
| | HCDR3: RETTTVGRYYYAMDY (SEQ ID | LCDR3: QQSTEDPWT (SEQ ID NO: |
| | NO: 31) | 34) |
| Merck | QVQLEQPGAEVVKPGASVKVSCKTSGY | QIVLTQSPATLSASPGEKATMTCSA |
| mAb | TFTSNWMHWVKQTPGKGLEWIGEIDPS | SSGVNYMHWYQQKPGTSPKRWIY |
| | DSYTNYNQKFDGKAKLTVDKSSSTAYM | DTDKTASGVPARFSGSGSGTSYSLT |
| | EVSDLTAEDSATYYCARGSNPYYYAMD | ISSMEAEDAATYYCHQRGSYTFGG |
| | YWGQGTSVTVSS (SEQ ID NO: 37) | GTKLEIK (SEQ ID NO: 38) |
| | HCDR1: SNWMH (SEQ ID NO: 39) | LCDR1: SASSGVNYMH (SEQ ID NO: |
| | HCDR2: EIDPSDSYTN (SEQ ID NO: 40) | 42) |
| | HCDR3: GSNPYYYAMDY (SEQ ID NO: | LCDR2: DTDKTAS (SEQ ID NO: 43) |
| | 41) | LCDR3: HQRGSYT (SEQ ID NO: 44) |
| Medarex | EVQLVQSGAEVKKPGESLKISCKGSGYS | AIQLTQSPSSLSASVGDRVTITCRAS |
| mAb | FSSSWIGWVRQMPGKGLEWMGIIYPDDS | QGISSALAWYQQKPGKAPKLLIYD |
| 21D4a | DTRYSPSFQGQVTISADKSIRTAYLQWSS | ASSLESGVPSRFSGSGSGTDFTLTISS |
| | LKASDTAMYYCARHVTMIWGVIIDFWG | LQPEDFATYYCQQFNSYPFTFGPGT |
| | QGTLVTVSS (SEQ ID NO: 45) | KVDIK (SEQ ID NO: 46) |
| | HCDR1: SSWIG (SEQ ID NO: 47) | LCDR1: RASQGISSALA (SEQ ID NO: |
| | HCDR2: IIYPDDSDTRYSPSFQG (SEQ ID | 50) |
| | NO: 48) | LCDR2: DASSLES (SEQ ID NO: 51) |
| | HCDR3: HVTMIWGVIIDF (SEQ ID NO: | LCDR3: QQFNSYPFT (SEQ ID NO: |
| | 49) | 52) |
| Medarex | EVQLVQSGAEVKKPGESLKISCKGSGYS | AIQLTQSPSSLSASVGDRVTITCRAS |
| mAb | FSSSWIGWVRQMPGKGLEWMGIIYPDDS | QGISSALAWYQQKPGKAPKLLIYD |
| 21D4 | DTRYSPSFQGQVTISADKSIRTAYLQWSS | ASSLESGVPSRFSGSGSGTDFTLTISS |
| | LKASDTAMYYCARHVTMIWGVIIDFWG | LQPEDFATYYCQQFNSYPYTFGQG |
| | QGTLVTVSS (SEQ ID NO: 45) | TKLEIK (SEQ ID NO: 53) |
| | HCDR1: SSWIG (SEQ ID NO: 47) | LCDR1: RASQGISSALA (SEQ ID NO: |
| | HCDR2: IIYPDDSDTRYSPSFQG (SEQ ID | 50) |
| | NO: 48) | LCDR2: DASSLES (SEQ ID NO: 51) |
| | HCDR3: HVTMIWGVIIDF (SEQ ID NO: | LCDR3: QQFNSYPYT (SEQ ID NO: |
| | 49) | 54) |
| Medarex | QVQLVQSGAEVKKPGSSVKVSCKDSGG | EIVLTQSPGTLSLSPGERATLSCRAS |
| mAb | TFSSYAISWVRQAPGQGLEWMGGIIPIFG | QSVSSSYLAWYQQKPGQAPRLLIY |
| 47G4 | TTNYAQQFQGRVTITADESTSTAYMELS | GASSRATGIPDRFSGSGSGTDFTLTI |
| | SLRSEDTAVYYCAREAVAADWLDPWG | SRLEPEDFAVYYCQQYGSSRFTFGP |
| | QGTLVTVSS (SEQ ID NO: 55) | GTKVDIK (SEQ ID NO: 56) |
| | HCDR1: SYAIS (SEQ ID NO: 57) | LCDR1: RASQSVSSSYLA (SEQ ID |
| | HCDR2: GIIPIFGTTNYAQQFQG (SEQ ID | NO: 60) |
| | NO: 58) | LCDR2: GASSRAT (SEQ ID NO: 61) |
| | HCDR3: EAVAADWLDP (SEQ ID NO: 59) | LCDR3: QQYGSSRFT (SEQ ID NO: |
| | | 62) |
| Medarex | EVQLVQSGAEVKKPGESLKISCKGSGYS | AIQLTQSPSSLSASVGDRVTITCRAS |
| mAb | FTSYWIAWVRQMPGKGLEWMGIIYPGD | QGISSALAWYQQKPGKAPKLLIYD |
| 27F3 | SDTRYSPSFQGQVTISADKSISTAYLQWS | ASSLESGVPSRFSGSGSGTDFTLTISS |
| | SLKASDTAMYYCARQGYSSGWDSYYG | LQPEDFATYYCQQFNSYPYTFGQG |
| | MGVWGQGTTVTVSS (SEQ ID NO: 63) | TKLEIK (SEQ ID NO: 64) |
| | HCDR1: SYWIA (SEQ ID NO: 65) | LCDR1: RASQGISSALA (SEQ ID NO: |
| | HCDR2: IIYPGDSDTRYSPSFQG (SEQ ID | 50) |
| | NO: 66) | LCDR2: DASSLES (SEQ ID NO: 51) |
| | HCDR3: QGYSSGWDSYYGMGV (SEQ ID | LCDR3: QQFNSYPYT (SEQ ID NO: |
| | NO: 67) | 54) |
| Medarex | QVQLVQSGAEVKKPGSSVKVSCKASGG | DIQMTQSPSSLSASVGDRVTITCRAS |
| mAb | TFSSYTINWVRQAPGQGLEWMGGIIPIFG | QGISSWLAWYQQKPEKAPKSLIYA |
| 3C10 | IPNYAQKFQGRVTITADESTNTAYMELS | ASSLQSGVPSRFSGSGSGTDFTLTIS |
| | SLRAEDTAVYYCARASGGSADYSYGMD | SLQPEDFATYYCQQYKRYPYTFGQ |
| | VWGQGTAVTVSS (SEQ ID NO: 68) | GTKLEIK (SEQ ID NO: 69) |
| | HCDR1: SYTIN (SEQ ID NO: 70) | LCDR1: RASQGISSWLA (SEQ ID |
| | HCDR2: GIIPIFGIPNYAQKFQG (SEQ ID | NO: 73) |
| | NO: 71) | LCDR2: AASSLQS (SEQ ID NO: 74) |
| | HCDR3: ASGGSADYSYGMDV (SEQ ID | LCDR3: QQYKRYPYT (SEQ ID NO: |
| | NO: 72) | 75) |
| Medarex | EVQLVQSGAEVKKPGESLNISCKGSGYS | AIQLTQSPSSLSASVGDRVTITCRAS |
| mAb 5G7 | FTSYWIGWVRQMPGKGLEWMGIIYPGD | QGISSALAWYQQKPGKAPKLLIYD |
| | SDTRYSPSFQGQVTISADKSINTAYLQWS | ASSLESGVPSRFSGSGSGTDFTLTISS |
| | SLKASDTAMYYCARGVSMIWGVIMDV | LQPEDFATYYCQQFNSYPWTFGQG |
| | WGQGTTVTVSS (SEQ ID NO: 76) | TKVEIK (SEQ ID NO: 77) |
| | HCDR1: SYWIG (SEQ ID NO: 78) | LCDR1: RASQGISSALA (SEQ ID NO: |
| | HCDR2: IIYPGDSDTRYSPSFQG (SEQ ID | 50) |
| | NO: 66) | LCDR2: DASSLES (SEQ ID NO: 51) |
| | HCDR3: GVSMIWGVIMDV (SEQ ID NO: | LCDR3: QQFNSYPWT (SEQ ID NO: |
| | 79) | 80) |
| Medarex | EVQLVQSGAEVKKPGESLQISCKGSGYT | AIQLTQSPSSLSASVGDRVTITCRAS |
| mAb | FTNYWIAWVRQMPGKGLEWMGIIYPGD | QGISSALAWYQQKPGKAPKLLIYD |
| 13F1 | SDTRYSPSFQGQVTISADKSISTAYLQWS | ASSLESGVPSRFSGSGSGTDFTLTISS |
| | GLKASDTAMYYCARQGYSSGWRSYYG | LQPEDFATYYCQQFNSYPHTFGQG |
| | MGVWGQGTTVTVSS (SEQ ID NO: 81) | TKLEIK (SEQ ID NO: 82) |
| | HCDR1: NYWIA (SEQ ID NO: 83) | LCDR1: RASQGISSALA (SEQ ID NO: |
| | HCDR2: IIYPGDSDTRYSPSFQG (SEQ ID | 50) |
| | NO: 66) | LCDR2: DASSLES (SEQ ID NO: 51) |
| | HCDR3: QGYSSGWRSYYGMGV (SEQ ID | LCDR3: QQFNSYPHT (SEQ ID NO: |
| | NO: 84) | 85) |
| Medarex | EVQLVQSGAEVKKPGESLQISCKGSGYT | AIQLTQSPSSLSASVGDRVTITCRAS |
| mAb | FTNYWIAWVRQMPGKGLEWMGIIYPGD | QGISSALAWYQQKPGKAPKLLIYD |
| 46E8 | SDTRYSPSFQGQVTISADKSISTAYLQWS | ASSLESGVPSRFSGSGSGTDFTLTISS |
| | GLKASDTAMYYCARQGYSSGWRSYYG | LQPEDFATYYCQQFNSYPHTFGQG |
| | MGVWGQGTTVTVSS (SEQ ID NO: 314) | TKLEIK (SEQ ID NO: 315) |
| | HCDR1: NYWIA (SEQ ID NO: 83) | LCDR1: RASQGISSALA (SEQ ID NO: |
| | HCDR2: IIYPGDSDTRYSPSFQG (SEQ ID | 50) |
| | NO: 66) | LCDR2: DASSLES (SEQ ID NO: 51) |
| | HCDR3: QGYSSGWRSYYGMGV (SEQ ID | LCDR3: QQFNSYPHT (SEQ ID NO: |
| | NO: 84) | 85) |
| Novimmune | EVQLVQSGAEVKKPGESLKISCKGSGYS | DIQMTQSPSSLSASVGDRVTITCRAS |
| mAb | FTSYWIGWVRQMPGKGLEWMGIIYPGD | QSISSYLNWYQQKPGKAPKLLIYAA |
| | SDTRYSPSFQGQVTISADKSISTAYLQWS | SSLQSGVPSRFSGSGSGTDFTLTISSL |
| | SLKASDTAMYYCARGVSGIYNLHGFDI | QPEDFATYYCQQGRFGSPFTFGQGT |
| | WGQGTLVTVSS (SEQ ID NO: 86) | KVEIK (SEQ ID NO: 87) |
| | HCDR1: GYSFTSYW (SEQ ID NO: 88) | LCDR1: QSISSY (SEQ ID NO: 91) |
| | HCDR2: IYPGDSDT (SEQ ID NO: 89) | LCDR2: AAS (SEQ ID NO: 92) |
| | HCDR3: ARGVSGIYNLHGFDI (SEQ ID | LCDR3: QQGRFGSPFT (SEQ ID NO: |
| | NO: 90) | 93) |
| Eureka | QVQLVETGGGLVQPGGSLRLSCAASGFT | QTVVTQEPSVSAAPGQKVTISCSGS |
| mAb-1 | FSSYAMSWVRQAPGKGLEWVSAISGSG | SSNIGNNYVSWYQQLPGTAPKLLIY |
| | GSTYYADSVKGRFTISRDNSKNTLYLQM | DNNKRPSGIPDRFSGSKSGTSATLGI |
| | NSLRAEDTAVYYCARYYYSRLDYWGQ | TGLQTGDEADYYCGTWDSSLSAGV |
| | GTLVTVSS (SEQ ID NO: 94) | FGTGTKLTVLGSR (SEQ ID NO: 95) |
| | scFv: | |
| | QTVVTQEPSVSAAPGQKVTISCSGSSSNIGNNYVSWYQQLPGTAPKLLIYDNNKR | |
| | PSGIPDRFSGSKSGTSATLGITGLQTGDEADYYCGTWDSSLSAGVFGTGTKLTVL | |
| | GSRGGGGSGGGGSGGGGSLEMAQVQLVETGGGLVQPGGSLRLSCAASGFTFSSY | |
| | AMSWVRQAPGKGLEWVSAISGSGGSTYYADSVKGRFTISRDNSKNTLYLQMNS | |
| | LRAEDTAVYYCARYYYSRLDYWGQGTLVTVSS (SEQ ID NO: 96) | |
| Eureka | QVQLVESGGGLVQPGGSLRLSCAASGFT | QSVLTQPPSVSAAPGQKVTISCSGSS |
| mAb-2 | FSSYAMSWVRQAPGKGLEWVSGISASG | SNIGNNYVSWYRQLPGTAPKLLIYE |
| | GSTYYADSVKGRFTISRDNSKNTLYLQM | NNKRPSGIPDRFSGSKSGTSATLGIT |
| | NSLRAEDTAVYYCARYYLSQIDSWGQG | GLQTGDEADYYCGTWDSSLRAGVF |
| | TLVTVSS (SEQ ID NO: 210) | GTGTKVTVL (SEQ ID NO: 211) |
| | scFv: | |
| | QSVLTQPPSVSAAPGQKVTISCSGSSSNIGNNYVSWYRQLPGTAPKLLIYENNKRP | |
| | SGIPDRFSGSKSGTSATLGITGLQTGDEADYYCGTWDSSLRAGVFGTGTKVTVLG | |
| | SRGGGGSGGGGSGGGGSLEMAQVQLVESGGGLVQPGGSLRLSCAASGFTFSSYA | |
| | MSWVRQAPGKGLEWVSGISASGGSTYYADSVKGRFTISRDNSKNTLYLQMNSL | |
| | RAEDTAVYYCARYYLSQIDSWGQGTLVTVSS (SEQ ID NO: 212) | |
| Eureka | EVQLVQSGAEVKKPGATVKISCKVSGYT | QSVLTQPPSASGTPGQRVTISCSGSS |
| mAb-3 | FTDYYMHWVQQAPGKGLEWMGLVDPE | SNIGSNTVNWYQQLPGTAPKLLIYS |
| | DGETIYAEKFQGRVTITADTSTDTAYME | NNQRPSGVPDRFSGSKSGTSASLAIS |
| | LSSLRSEDTAVYYCATGIYSRPLGYWGQ | GLQSEDEADYYCAAWDDSLNGHV |
| | GTLVTVSS (SEQ ID NO: 213) | VFGGGTKLTVL (SEQ ID NO: 214) |
| | scFv: | |
| | QSVLTQPPSASGTPGQRVTISCSGSSSNIGSNTVNWYQQLPGTAPKLLIYSNNQRP | |
| | SGVPDRFSGSKSGTSASLAISGLQSEDEADYYCAAWDDSLNGHVVFGGGTKLTV | |
| | LGSRGGGGSGGGGSGGGGSLEMAEVQLVQSGAEVKKPGATVKISCKVSGYTFT | |
| | DYYMHWVQQAPGKGLEWMGLVDPEDGETIYAEKFQGRVTITADTSTDTAYME | |
| | LSSLRSEDTAVYYCATGIYSRPLGYWGQGTLVTVSS (SEQ ID NO: 215) | |
| Eureka | EVQLVETGGGLVQPGGSLRLSCAASGFT | SYVLTQPPSASGTPGQRVTISCSGSS |
| mAb-4 | FSSYAMSWVRQAPGKGLEWVSAISGSG | SNIGSHTVNWYQQLPGTAPKLLIYS |
| | GSTYYADSVKGRFTISRDNSKNTLYLQM | NNQRPSGVPDRFSGSKSGTSASLAIS |
| | NSLRAEDTAVYYCARSDGKHFWQQYD | GLQSEDEADYYCAAWDDSLNGYV |
| | AWGQGTLVTVSS (SEQ ID NO: 216) | FGTGTKVTVL (SEQ ID NO: 217) |
| | scFv: | |
| | SYVLTQPPSASGTPGQRVTISCSGSSSNIGSHTVNWYQQLPGTAPKLLIYSNNQRP | |
| | SGVPDRFSGSKSGTSASLAISGLQSEDEADYYCAAWDDSLNGYVFGTGTKVTVL | |
| | GSRGGGGSGGGGSGGGGSLEMAEVQLVETGGGLVQPGGSLRLSCAASGFTFSSY | |
| | AMSWVRQAPGKGLEWVSAISGSGGSTYYADSVKGRFTISRDNSKNTLYLQMNS | |
| | LRAEDTAVYYCARSDGKHFWQQYDAWGQGTLVTVSS (SEQ ID NO: 218) | |
| Eureka | EVQLVESGGGLVQPGGSLRLSCAASGFT | DIQLTQSPSSLSAYVGDRVTITCRAS |
| mAb-5 | VSSNYMSWVRQAPGKGLEWVSAISGSG | QGITNSLAWYQQKPGKAPKLLLHA |
| | GSTYYADSVKGRFTISRDNSKNTLYLQM | ASRLESGVPSRFSGSGFGTDFTLTIS |
| | NSLRAEDTAVYYCARMNIDYWGQGTLV | SLQPEDFAVYYCQHYLGTPYSFGQ |
| | TVSS (SEQ ID NO: 219) | GTKVEIK (SEQ ID NO: 220) |
| | scFv: | |
| | DIQLTQSPSSLSAYVGDRVTITCRASQGITNSLAWYQQKPGKAPKLLLHAASRLE | |
| | SGVPSRFSGSGFGTDFTLTISSLQPEDFAVYYCQHYLGTPYSFGQGTKVEIKRSRG | |
| | GGGSGGGGSGGGGSLEMAEVQLVESGGGLVQPGGSLRLSCAASGFTVSSNYMS | |
| | WVRQAPGKGLEWVSAISGSGGSTYYADSVKGRFTISRDNSKNTLYLQMNSLRAE | |
| | DTAVYYCARMNIDYWGQGTLVTVSS (SEQ ID NO: 221) | |
| Eureka | EVQLVQSGAEVKRPGESLTISCKGSEYSF | EIVLTQSPSSLSASVGDRVTISCRAS |
| mAb-6 | ASYWITWVRQMPGKGLEWMGRIDPSDS | QSVSRFLNWYQQKPGKAPKLLIYG |
| | YTNYSPSFQGHVTISADKSISTAYLQWSS | VSTLERGVPSRFSGSGSGTDFTLTIS |
| | LKASDTAIYYCARPFQYDYGGYSDAFDI | SLQPEDFATYYCQESYIIPLTFGGGT |
| | WGQGTMVTVSS (SEQ ID NO: 222) | KLEIK (SEQ ID NO: 223) |
| | scFv: | |
| | EIVLTQSPSSLSASVGDRVTISCRASQSVSRFLNWYQQKPGKAPKLLIYGVSTLER | |
| | GVPSRFSGSGSGTDFTLTISSLQPEDFATYYCQESYIIPLTFGGGTKLEIKRSRGGG | |
| | GSGGGGSGGGGSLEMAEVQLVQSGAEVKRPGESLTISCKGSEYSFASYWITWVR | |
| | QMPGKGLEWMGRIDPSDSYTNYSPSFQGHVTISADKSISTAYLQWSSLKASDTAI | |
| | YYCARPFQYDYGGYSDAFDIWGQGTMVTVSS (SEQ ID NO: 224) | |
| Eureka | QMQLVQSGAEVKKAGSSVKVSCETSGG | EIVMTQSPLSLSVTPGEPASISCRSS |
| mAb-7 | TFSSSSVNWVRQAPGQGLEWMGGIIPIV | QSLLDSNGFNSLDWYLQKPGQSPQ |
| | GTPNYAQKFQDRVTITAVESTFTAYMEL | LLIHLGSDRASGVPDRFSGSGSGTD |
| | SGLRSEDTAVYYCARGGYRDYMDVWG | FTLKISRVEAEDVGIYYCMQSLQIPT |
| | RGTTVTVSS (SEQ ID NO: 225) | FGQGTKVEIK (SEQ ID NO: 226) |
| | scFv: | |
| | EIVMTQSPLSLSVTPGEPASISCRSSQSLLDSNGFNSLDWYLQKPGQSPQLLIHLGS | |
| | DRASGVPDRFSGSGSGTDFTLKISRVEAEDVGIYYCMQSLQIPTFGQGTKVEIKRS | |
| | RGGGGSGGGGSGGGGSLEMAQMQLVQSGAEVKKAGSSVKVSCETSGGTFSSSS | |
| | VNWVRQAPGQGLEWMGGIIPIVGTPNYAQKFQDRVTITAVESTFTAYMELSGLR | |
| | SEDTAVYYCARGGYRDYMDVWGRGTTVTVSS (SEQ ID NO: 227) | |
| Eureka | EVQLVESGGGLIQPGGSLRLSCAASGFT | SYELTQPPSASGTPGQRVTISCSGSS |
| mAb-8 | VSSNYMSWVRQAPGKGLEWVSVIYSGG | SNIGSNYVYWYQQLPGTAPKLLIYR |
| | STYYADSVKGRFTISRDNSKNTLYLQMN | NNQRPSGVPDRFSGSKSGTSASLAIS |
| | SLRAEDTAVYYCARGGFGAEFDYWGQG | GLRSEDEADYYCAAWDDSLSGYVF |
| | TLVTVSS (SEQ ID NO: 228) | GTGTKVTVL (SEQ ID NO: 229) |
| | scFv: | |
| | SYELTQPPSASGTPGQRVTISCSGSSSNIGSNYVYWYQQLPGTAPKLLIYRNNQRP | |
| | SGVPDRFSGSKSGTSASLAISGLRSEDEADYYCAAWDDSLSGYVFGTGTKVTVL | |
| | GSRGGGGSGGGGSGGGGSLEMAEVQLVESGGGLIQPGGSLRLSCAASGFTVSSN | |
| | YMSWVRQAPGKGLEWVSVIYSGGSTYYADSVKGRFTISRDNSKNTLYLQMNSL | |
| | RAEDTAVYYCARGGFGAEFDYWGQGTLVTVSS (SEQ ID NO: 230) | |
| Eureka | EVQLVESGGGLIQPGGSLRLSCAASGFT | SYVLTQPPSVSVSPGQTASITCSGDK |
| mAb-9 | VSSNYMSWVRQAPGKGLEWVSVIYSGG | LGDKYASWYQQKPGQSPVLVIYQD |
| | STYYADSVKGRFTISRDNSKNTLYLQMN | NKRPSGIPERFSGSNSGNTATLTISG |
| | SLRAEDTAVYYCARGGISDDYYGSGSY | TQAMDEADYYCQAWDSSTEDVFG |
| | DNWGQGTLVTVSS (SEQ ID NO: 231) | PGTKVTVL (SEQ ID NO: 232) |
| | scFv: | |
| | SYVLTQPPSVSVSPGQTASITCSGDKLGDKYASWYQQKPGQSPVLVIYQDNKRPS | |
| | GIPERFSGSNSGNTATLTISGTQAMDEADYYCQAWDSSTEDVFGPGTKVTVLGSR | |
| | GGGGSGGGGSGGGGSLEMAEVQLVESGGGLIQPGGSLRLSCAASGFTVSSNYMS | |
| | WVRQAPGKGLEWVSVIYSGGSTYYADSVKGRFTISRDNSKNTLYLQMNSLRAE | |
| | DTAVYYCARGGISDDYYGSGSYDNWGQGTLVTVSS (SEQ ID NO: 233) | |
| Eureka | EVQLVESGGGLVQPGGSLRLSCAASGFT | DIQLTQSPSSLSASVGDRVTITCRAS |
| mAb-10 | VSSNYMSWVRQAPGKGLEWVSVIYSGG | QSISSYLNWYQQKPGKAPKLLIYAA |
| | STYYADSVKGRFTISRDNSKNTLYLQMN | SSLQSGVPSRFSGSGSGTDFTLTISSL |
| | SLRAEDTAVYYCARERGMGYAFDIWGQ | QPEDFATYYCQQSYSTPFTFGGGTK |
| | GTMVTVSS (SEQ ID NO: 234) | VEIK (SEQ ID NO: 235) |
| | scFv: | |
| | DIQLTQSPSSLSASVGDRVTITCRASQSISSYLNWYQQKPGKAPKLLIYAASSLQS | |
| | GVPSRFSGSGSGTDFTLTISSLQPEDFATYYCQQSYSTPFTFGGGTKVEIKRSRGG | |
| | GGSGGGGSGGGGSLEMAEVQLVESGGGLVQPGGSLRLSCAASGFTVSSNYMSW | |
| | VRQAPGKGLEWVSVIYSGGSTYYADSVKGRFTISRDNSKNTLYLQMNSLRAEDT | |
| | AVYYCARERGMGYAFDIWGQGTMVTVSS (SEQ ID NO: 236) | |
| Eureka | QLQLQESGPGLVKPSETLSLTCSVSGVS | DIQMTQSPSSLSASVGDRVTITCRAS |
| mAb-11 | MSENYWSWIRQPPGKRLEWIGCAHYTG | QGIGSYLAWYQQKPGKAPKLLIYP |
| | DTHYNPSLKGRVTISLDTSMNQFSLRLN | ASTLQSGVPSRFSGSGSGTEFTLTIS |
| | SVTAADTAVYYCASYHPFNYWGQGTLV | SLQPEDFATYYCQQLNSLFGQGTRL |
| | TVSS (SEQ ID NO: 237) | EIK (SEQ ID NO: 238) |
| | scFv: | |
| | DIQMTQSPSSLSASVGDRVTITCRASQGIGSYLAWYQQKPGKAPKLLIYPASTLQS | |
| | GVPSRFSGSGSGTEFTLTISSLQPEDFATYYCQQLNSLFGQGTRLEIKRSRGGGGS | |
| | GGGGSGGGGSLEMAQLQLQESGPGLVKPSETLSLTCSVSGVSMSENYWSWIRQP | |
| | PGKRLEWIGCAHYTGDTHYNPSLKGRVTISLDTSMNQFSLRLNSVTAADTAVYY | |
| | CASYHPFNYWGQGTLVTVSS (SEQ ID NO: 239) | |
| Eureka | EVQLVQSGAEVRRPGATVKISCKVSGYT | QAVLTQPPSASGTPGQRVTISCSGSS |
| mAb-12 | FNDFYLHWVRQAPGKGLEWMGRIDPED | SNIGTKTVNWYQVLPGTAPKLLIYS |
| | GKTRYAEKFQGRLTITADTSTDTLYMQL | NYRRPSGVPDRFSGSKSGTSASLAIS |
| | GGLTSDDTAVYYCTTDWGYSSSLREEDI | GLQSDDEADYYCALWDDSLDGYV |
| | WYDCWGQGTLVTVSS (SEQ ID NO: 240) | FGTGTKVTVL (SEQ ID NO: 241) |
| | scFv: | |
| | QAVLTQPPSASGTPGQRVTISCSGSSSNIGTKTVNWYQVLPGTAPKLLIYSNYRRP | |
| | SGVPDRFSGSKSGTSASLAISGLQSDDEADYYCALWDDSLDGYVFGTGTKVTVL | |
| | GSRGGGGSGGGGSGGGGSLEMAEVQLVQSGAEVRRPGATVKISCKVSGYTFND | |
| | FYLHWVRQAPGKGLEWMGRIDPEDGKTRYAEKFQGRLTITADTSTDTLYMQLG | |
| | GLTSDDTAVYYCTTDWGYSSSLREEDIWYDCWGQGTLVTVSS (SEQ ID NO: | |
| | 242) | |
| Eureka | EVQLVQSGAEVKKPGSSVKVSCKASGG | SYELTQPPSVSVAPGKTARITCGGN |
| mAb-13 | TFSSYAISWVRQAPGQGLEWMGGIIPIFG | NIGSKSVHWYQQKPGQAPVLVIYY |
| | TANYAQKFQGRVTITADESTSTAYMELS | DSDRPSGIPERFSGSNSGNTATLTIS |
| | SLRSEDTAVYYCARDYGYGDYGDAFDI | RVEAGDEADYYCQVWDSSSDHYV |
| | WGQGTMVTVSS (SEQ ID NO: 243) | FGTGTKVTVL (SEQ ID NO: 244) |
| | scFv: | |
| | SYELTQPPSVSVAPGKTARITCGGNNIGSKSVHWYQQKPGQAPVLVIYYDSDRPS | |
| | GIPERFSGSNSGNTATLTISRVEAGDEADYYCQVWDSSSDHYVFGTGTKVTVLGS | |
| | RGGGGSGGGGSGGGGSLEMAEVQLVQSGAEVKKPGSSVKVSCKASGGTESSYAI | |
| | SWVRQAPGQGLEWMGGIIPIFGTANYAQKFQGRVTITADESTSTAYMELSSLRSE | |
| | DTAVYYCARDYGYGDYGDAFDIWGQGTMVTVSS (SEQ ID NO: 245) | |
| Eureka | EVQLVQSGAEVKKPGESLKISCKGSGYS | SYVLTQPPSVSVAPGKTARITCGGN |
| mAb-14 | FTSYWIGWVRQMPGKGLEWMGIIYPGD | NIGSKSVHWYQQRPGQAPVLVVYD |
| | SDTRYSPSFQGQVTISADKSISTAYLQWS | DSDRPSGIPERFSGSNSGNTATLTIS |
| | SLKASDTAMYYCARVVGTIYSMQYDV | RVEAGDEADYSCQVWDSSSDHYVF |
| | WGQGTLVTVSS (SEQ ID NO: 246) | GPGTKVTVL (SEQ ID NO: 247) |
| | scFv: | |
| | SYVLTQPPSVSVAPGKTARITCGGNNIGSKSVHWYQQRPGQAPVLVVYDDSDRP | |
| | SGIPERFSGSNSGNTATLTISRVEAGDEADYSCQVWDSSSDHYVFGPGTKVTVLG | |
| | SRGGGGSGGGGSGGGGSLEMAEVQLVQSGAEVKKPGESLKISCKGSGYSFTSYW | |
| | IGWVRQMPGKGLEWMGIIYPGDSDTRYSPSFQGQVTISADKSISTAYLQWSSLKA | |
| | SDTAMYYCARVVGTIYSMQYDVWGQGTLVTVSS (SEQ ID NO: 248) | |
| Eureka | EVQLVQSGAEVKKPGESLKISCKGSGYS | LPVLTQPPSVSVAPGKTARITCGGN |
| mAb-15 | FTSYWIGWVRQMPGKGLEWMGIIYPGD | NIGSKSVHWYQQKPGQAPVLVVYD |
| | SDTRYSPSFQGQVTISADKSISTAYLQWS | DSDRPSGIPERFSGSNSGNTATLTIS |
| | SLKASDTAMYYCARQVWGWQGGMYP | RVEAGDEADYYCQVWDSSSDYVV |
| | RSNWWYNMDSWGQGTLVTVSS (SEQ | FGGGTKLTVL (SEQ ID NO: |
| | ID NO: 249) | 250) |
| | scFv: | |
| | LPVLTQPPSVSVAPGKTARITCGGNNIGSKSVHWYQQKPGQAPVLVVYDDSDRP | |
| | SGIPERFSGSNSGNTATLTISRVEAGDEADYYCQVWDSSSDYVVFGGGTKLTVLG | |
| | SRGGGGSGGGGSGGGGSLEMAEVQLVQSGAEVKKPGESLKISCKGSGYSFTSYW | |
| | IGWVRQMPGKGLEWMGIIYPGDSDTRYSPSFQGQVTISADKSISTAYLQWSSLKA | |
| | SDTAMYYCARQVWGWQGGMYPRSNWWYNMDSWGQGTLVTVSS (SEQ ID | |
| | NO: 251) | |
| Eureka | EVQLVQSGAEVKKPGESLKISCKGSGYS | QAVLTQPPSVSEAPRQRVTISCSGSS |
| mAb-16 | FTSYWIGWVRQMPGKGLEWMGIIYPGD | SNVGNNAVNWYQQVPGKAPKLLI |
| | SDTRYSPSFQGQVTISADKSISTAYLQWS | YYDDLLSSGVSDRFSGSKSGTSASL |
| | SLKASDTAMYYCARWSSTWDSMYMDY | AISGLQSEDEADYYCAAWDDSLNG |
| | WGQGTLVTVSS (SEQ ID NO: 252) | PVFGGGTKLTVL (SEQ ID NO: 253) |
| | scFv: | |
| | QAVLTQPPSVSEAPRQRVTISCSGSSSNVGNNAVNWYQQVPGKAPKLLIYYDDL | |
| | LSSGVSDRFSGSKSGTSASLAISGLQSEDEADYYCAAWDDSLNGPVFGGGTKLTV | |
| | LGSRGGGGSGGGGSGGGGSLEMAEVQLVQSGAEVKKPGESLKISCKGSGYSFTS | |
| | YWIGWVRQMPGKGLEWMGIIYPGDSDTRYSPSFQGQVTISADKSISTAYLQWSS | |
| | LKASDTAMYYCARWSSTWDSMYMDYWGQGTLVTVSS (SEQ ID NO: 254) | |
| Eureka | EVQLVQSGAEVKKPGESLRISCKGSGYS | QPVLTQPPSVSVAPGKTARITCGGN |
| mAb-17 | FTSYWIGWVRQMPGKGLEWMGIIYPGD | NIGSESVHWYQQKPGQAPMVVIYY |
| | SDTRYSPSFQGQVTISADKSISTAYLQWS | DSNRPSGIPERFSGSNSGNTATLTVS |
| | SLKASDTAMYYCARVTYSMDSYYFDSW | RVEAEDEADYYCQVWNSSSDHRG |
| | GQGTLVTVSS (SEQ ID NO: 255) | VFGGGTKLTV (SEQ ID NO: 256) |
| | scFv: | |
| | QPVLTQPPSVSVAPGKTARITCGGNNIGSESVHWYQQKPGQAPMVVIYYDSNRP | |
| | SGIPERFSGSNSGNTATLTVSRVEAEDEADYYCQVWNSSSDHRGVFGGGTKLTV | |
| | LGSRGGGGSGGGGSGGGGSLEMAEVQLVQSGAEVKKPGESLRISCKGSGYSFTS | |
| | YWIGWVRQMPGKGLEWMGIIYPGDSDTRYSPSFQGQVTISADKSISTAYLQWSS | |
| | LKASDTAMYYCARVTYSMDSYYFDSWGQGTLVTVSS (SEQ ID NO: 257) | |
| WuXi | EVQLQQSGPELVKPGASVKMSCKASGY | DAVMTQTPLSLPVSLGDQASISCRS |
| WBP7011- | TFTNYVIHWVKQKPGQGLEWIGYFNPY | SQSLENSNGNTYLNWYLQKPGQSP |
| 4.34.11 | NDGTEYNEKFKAKATLTSDKSSSTAYM | QLLIYRVSNRFSGVLDRFSGSGSGT |
| | ELSSLTSEDSAVYYCAKGPYYYGSSPFD | DFTLKISRVEAEDLGVYFCLQVTHV |
| | YWGQGTTLTVSS (SEQ ID NO: 316) | PYTFGGGTKLEIK (SEQ ID NO: 317) |
| | HCDR1: GYTFTNYVIH (SEQ ID NO: 318) | LCDR1: RSSQSLENSNGNTYLN |
| | HCDR2: YFNPYNDGTEYNEKFKA (SEQ | (SEQ ID NO: 321) |
| | ID NO: 319) | LCDR2: RVSNRFS (SEQ ID NO: 322) |
| | HCDR3: GPYYYGSSPFDY (SEQ ID NO: | LCDR3: RVSNRFS (SEQ ID NO: 323) |
| | 320) | |
| WuXi | QVQLQQSGAELVRPGSSVKISCKASGYA | DIQMTQTTSSLSASLGDRVTISCRAS |
| WBP7011- | FSTYWMNWVKQRPGQGLEWIGQIYPGD | QDISNYLNWYQQKPDGTVKLLIYY |
| 4.87.6 | DDTKYNGKFKGKASLTADKSSSTAYMQ | TSRLHSGVPARFSGSGSGTDYSLTIS |
| | LISLTSEDSAVYFCARRYFRYDYWYSDV | NLEQEDIATYFCHQGNTLPLTFGAG |
| | WGAGTTVTVTS (SEQ ID NO: 324) | TKLELK (SEQ ID NO: 325) |
| | HCDR1: GYAFSTYWMN (SEQ ID NO: | LCDR1: RASQDISNYLN (SEQ ID |
| | 326) | NO: 329) |
| | HCDR2: QIYPGDDDTKYNGKFKG (SEQ | LCDR2: YTSRLHS (SEQ ID NO: 330) |
| | ID NO: 327) | |
| | HCDR3: RYFRYDYWYSDV (SEQ ID NO: | LCDR3: HQGNTLPLT (SEQ ID NO: |
| | 328) | 331) |
| WuXi | EIQLQQSGPELVKPGASVKVSCKASGYA | QIVLTQSPAIMSASLGEEITLTCSASS |
| WBP7011_ | FTSYNMYWVKQSHGKSLEWIGYIDPYN | TVNYMHWYQQKSGTSPKLLIYSTS |
| 4.155.8 | GDTTYNQKFKGKATLTVDKSSSTAYMH | NLASGVPSRFSGSGSGTFYSLTIRSV |
| | LNSLTSEDSAVYYCLTTAYAMDYWGQG | EAEDAADYYCHQWSSYPYTFGGGT |
| | TSVTVSS (SEQ ID NO: 332) | KLEIK (SEQ ID NO: 333) |
| | HCDR1: GYAFTSYNMY (SEQ ID NO: 334) | LCDR1: SASSTVNYMH (SEQ ID NO: |
| | HCDR2: YIDPYNGDTTYNQKFKG (SEQ | 337) |
| | ID NO: 335) | LCDR2: STSNLAS (SEQ ID NO: 338) |
| | HCDR3: TAYAMDY (SEQ ID NO: 336) | LCDR3: HQWSSYPYT (SEQ ID NO: |
| | | 339) |
| WuXi | QVQLVQSGAEVKKPGSSVKVSCKASGY | DIVMTQTPLSLPVTPGEPASISCRSS |
| WBP7011- | TFTDYVIHWVRQAPGQGLEWMGYFNPY | QSLENSNHNTYINWYLQKPGQSPQ |
| 4.34.11- | NDGTEYNEKFKARVTITADKSTSTAYME | LLIYRVSKRFSGVPDRFSGSGSGTDF |
| z1-m5 | LSSLRSEDTAVYYCARGPYYYGSSPFDY | TLKISRVEAEDVGVYYCHQVTHVP |
| | WGQGTTVTVSS (SEQ ID NO: 340) | YTFGQGTKLEIK (SEQ ID NO: 344) |
| | HCDR1: GYTFTNYVIH (SEQ ID NO: 341) | LCDR1: RSSQSLENSNHNTYIN (SEQ |
| | HCDR2: YFNPYNDGTEYNEKFKA (SEQ | ID NO: 345) |
| | ID NO: 342) | LCDR2: RVSKRFS (SEQ ID NO: 346) |
| | HCDR3: GPYYYGSSPFDY (SEQ ID NO: | LCDR3: HQVTHVPYT (SEQ ID NO: |
| | 343) | 347) |
| WuXi | QVQLVQSGAEVKKPGASVKVSCKASGY | DIQMTQSPSSLSASVGDRVTITCRAS |
| WBP7011- | AFSTYWMNWVRQAPGQGLEWMGQIYP | QDISNYLNWYQQKPGKVPKLLIYY |
| 4.87.6- | GDDDTKYSGKFKGRVTITADKSTSTAY | TSRLHSGVPSRFSGSGSGTDFTLTIS |
| z1(N-S) | MELSSLRSEDTAVYYCARRYFRYDYWY | SLQPEDVATYYCHQGNTLPLTFGQ |
| | SDVWGQGTTVTVSS (SEQ ID NO: 348) | GTKLEIK (SEQ ID NO: 349) |
| | HCDR1: GYAFSTYWMN (SEQ ID NO: | LCDR1: RASQDISNYLN (SEQ ID |
| | 350) | NO: 353) |
| | HCDR2: QIYPGDDDTKYSGKFKG (SEQ | LCDR2: YTSRLHS (SEQ ID NO: 354) |
| | ID NO: 351) | LCDR3: HQGNTLPLT (SEQ ID NO: |
| | HCDR3: RYFRYDYWYSDV (SEQ ID NO: | 355) |
| | 352) | |
| WuXi | QMQLVQSGPEVKKPGTSVKVSCKASGY | DIQLTQSPSFLSASVGDRVTITCSAS |
| WBP7011_ | AFTSYNMYWVRQARGQRLEWIGYIDPY | STVNYMHWYQQKPGKAPKLLIYST |
| 4.155.8- | NADTTYNQKFKGRVTITRDMSTSTAYM | SNLASGVPSRFSGSGSGTEFTLTISS |
| z1-P15 | ELSSLRSEDTAVYYCLTTAYAMDYWGQ | LQPEDFATYYCHQWSSYPYTFGQG |
| | GTLVTVSS (SEQ ID NO: 356) | TKLEIK (SEQ ID NO: 357) |
| | HCDR1: GYAFTSYNMY (SEQ ID NO: 358) | LCDR1: SASSTVNYMH (SEQ ID NO: |
| | HCDR2: YIDPYNADTTYNQKFKG (SEQ | 361) |
| | ID NO: 359) | LCDR2: STSNLAS (SEQ ID NO: 362) |
| | HCDR3: TAYAMDY (SEQ ID NO: 360) | LCDR3: HQWSSYPYT (SEQ ID NO: |
| | | 363) |
| Legend | QVKLEESGGELVQPGGPLRLSCAASGNI | N/A |
| mAb | FSINRMGWYRQAPGKQRAFVASITVRGI | |
| | TNYADSVKGRFTISVDKSKNTIYLQMNA | |
| | LKPEDTAVYYCNAVSSNRDPDYWGQGT | |
| | QVTVSS (SEQ ID NO: 364) | |
| | HCDR1: INRMG (SEQ ID NO: 365) | |
| | HCDR2: SITVRGITNYADSVKG (SEQ ID | |
| | NO: 366) | |
| | HCDR3: VSSNRDPDY (SEQ ID NO: 367) | |

Where the VL and LCDR sequences are noted as “N/A,“ the antigen-binding site is an sdAb having a VH (e.g., VHH) only.

In certain embodiments, the first antigen-binding site comprises a VH comprising complementarity determining regions HCDR1, HCDR2, and HCDR3, and a VL comprising complementarity determining regions LCDR1, LCDR2, and LCDR3. In certain embodiments, the VH comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to the VH of an antibody provided in Table 1, and the VL comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to the VL of the antibody provided in Table 1. In certain embodiments, the VH comprises the HCDR1, HCDR2, and HCDR3 sequences of the antibody provided in Table 1, and the VL comprises the LCDR1, LCDR2, and LCDR3 sequences of the antibody provided in Table 1. In certain embodiments, the VH comprises the amino acid sequence of the VH of an antibody provided in Table 1, and the VL comprises the amino acid sequence of the VL of the antibody provided in Table 1.

In certain embodiments, the VH of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 1, and the VL of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 2. In certain embodiments, the VH comprises HCDR1, HCDR2, and HCDR3 sequences set forth in SEQ ID NOs: 3, 4, and 5, respectively, and the VL comprises LCDR1, LCDR2, and LCDR3 sequences set forth in SEQ ID NOs: 6, 7, and 8, respectively. In certain embodiments, the VH comprises the amino acid sequence set forth in SEQ ID NO: 1, and the VL comprises the amino acid sequence set forth in SEQ ID NO: 2.

In certain embodiments, the VH of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 10, and the VL of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 11. In certain embodiments, the VH comprises HCDR1, HCDR2, and HCDR3 sequences set forth in SEQ ID NOs: 12, 13, and 14, respectively, and the VL comprises LCDR1, LCDR2, and LCDR3 sequences set forth in SEQ ID NOs: 15, 16, and 17, respectively. In certain embodiments, the VH comprises the amino acid sequence set forth in SEQ ID NO: 10, and the VL comprises the amino acid sequence set forth in SEQ ID NO: 11.

In certain embodiments, the VH of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 19, and the VL of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 20. In certain embodiments, the VH comprises HCDR1, HCDR2, and HCDR3 sequences set forth in SEQ ID NOs: 21, 22, and 23, respectively, and the VL comprises LCDR1, LCDR2, and LCDR3 sequences set forth in SEQ ID NOs: 24, 25, and 26, respectively. In certain embodiments, the VH comprises the amino acid sequence set forth in SEQ ID NO: 19, and the VL comprises the amino acid sequence set forth in SEQ ID NO: 20.

In certain embodiments, the VH of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 27, and the VL of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 28. In certain embodiments, the VH comprises HCDR1, HCDR2, and HCDR3 sequences set forth in SEQ ID NOs: 29, 30, and 31, respectively, and the VL comprises LCDR1, LCDR2, and LCDR3 sequences set forth in SEQ ID NOs: 32, 33, and 34, respectively. In certain embodiments, the VH comprises the amino acid sequence set forth in SEQ ID NO: 27, and the VL comprises the amino acid sequence set forth in SEQ ID NO: 28.

In certain embodiments, the VH of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 35, and the VL of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 36. In certain embodiments, the VH comprises HCDR1, HCDR2, and HCDR3 sequences set forth in SEQ ID NOs: 29, 30, and 31, respectively, and the VL comprises LCDR1, LCDR2, and LCDR3 sequences set forth in SEQ ID NOs: 32, 33, and 34, respectively. In certain embodiments, the VH comprises the amino acid sequence set forth in SEQ ID NO: 35, and the VL comprises the amino acid sequence set forth in SEQ ID NO: 36.

In certain embodiments, the VH of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 37, and the VL of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 38. In certain embodiments, the VH comprises HCDR1, HCDR2, and HCDR3 sequences set forth in SEQ ID NOs: 39, 40, and 41, respectively, and the VL comprises LCDR1, LCDR2, and LCDR3 sequences set forth in SEQ ID NOs: 42, 43, and 44, respectively. In certain embodiments, the VH comprises the amino acid sequence set forth in SEQ ID NO: 37, and the VL comprises the amino acid sequence set forth in SEQ ID NO: 38.

In certain embodiments, the VH of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 45, and the VL of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 46. In certain embodiments, the VH comprises HCDR1, HCDR2, and HCDR3 sequences set forth in SEQ ID NOs: 47, 48, and 49, respectively, and the VL comprises LCDR1, LCDR2, and LCDR3 sequences set forth in SEQ ID NOs: 50, 51, and 52, respectively. In certain embodiments, the VH comprises the amino acid sequence set forth in SEQ ID NO: 45, and the VL comprises the amino acid sequence set forth in SEQ ID NO: 46.

In certain embodiments, the VH of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 86, and the VL of the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 87. In certain embodiments, the VH comprises HCDR1, HCDR2, and HCDR3 sequences set forth in SEQ ID NOs: 88, 89, and 90, respectively, and the VL comprises LCDR1, LCDR2, and LCDR3 sequences set forth in SEQ ID NOs: 91, 92, and 93, respectively. In certain embodiments, the VH comprises the amino acid sequence set forth in SEQ ID NO: 86, and the VL comprises the amino acid sequence set forth in SEQ ID NO: 87.

Such antigen-binding site may take the form of scFv. In certain embodiments, the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to an scFv sequence provided in Table 1. In certain embodiments, the first antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 9, 18, 96, 212, 215, 218, 221, 224, 227, 230, 233, 236, 239, 242, 245, 248, 251, 254, or 257.

In other embodiments, the first antigen-binding site comprises an sdAb comprising a VH comprising complementarity determining regions HCDR1, HCDR2, and HCDR3. In certain embodiments, the VH comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to the VH of an sdAb antibody provided in Table 1. In certain embodiments, the VH comprises the HCDR1, HCDR2, and HCDR3 sequences of the antibody provided in Table 1. In certain embodiments, the VH comprises the amino acid sequence of the VH of an sdAb provided in Table 1.

In certain embodiments, the first antigen-binding site competes for binding CD19 (e.g., human CD19) with an antibody or antigen-binding fragment thereof comprising the VH, VL and/or scFv sequences provided in Table 1.

In certain embodiments, the first antigen-binding site of the multi-specific binding protein binds CD19 (e.g., human CD19) with a dissociation constant (K_{D}) of about 10 pM-about 1 μM. The K_{D} can be measured by a method known in the art. In certain embodiments, the K_{D} is measured by SPR to CD19 or an extracellular fragment thereof immobilized on a chip. In certain embodiments, the K_{D} is measured by flow cytometry to CD19 expressed on the surface of cells, for example, following the method described in Example 5 below.

In certain embodiments, the first antigen-binding site binds CD19 with a K_{D} of lower than or equal to 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 90 pM, 80 pM, 70 pM, 60 pM, 50 pM, 40 pM, 30 pM, 20 pM, or 10 pM. For example, in certain embodiments, the first antigen-binding site binds CD19 with a K_{D} of about 10 pM-about 1 nM, about 10 pM
about 0.9 nM, about 10 pM-about 0.8 nM, about 10 pM-about 0.7 nM, about 10 pM-about 0.6 nM, about 10 pM nM-about 0.5 nM, about 10 pM-about 0.4 nM, about 10 pM-about 0.3 nM, about 10 pM-about 0.2 nM, about 10 pM-about 0.1 nM, about 10 pM-about 50 pM, 0.1 nM-about 10 nM, about 0.1 nM-about 9 nM, about 0.1 nM-about 8 nM, about 0.1 nM-about 7 nM, about 0.1 nM-about 6 nM, about 0.1 nM-about 5 nM, about 0.1 nM-about 4 nM, about 0.1 nM-about 3 nM, about 0.1 nM-about 2 nM, about 0.1 nM-about 1 nM, about 0.1 nM-about 0.5 nM, about 0.5 nM-about 10 nM, about 1 nM-about 10 nM, about 2 nM-about 10 nM, about 3 nM-about 10 nM, about 4 nM-about 10 nM, about 5 nM-about 10 nM, about 6 nM-about 10 nM, about 7 nM-about 10 nM, about 8 nM-about 10 nM, or about 9 nM-about 10 nM.

In certain embodiments, the first antigen-binding site binds CD19 with a K_{D} greater than or equal to 10 nM, 20 nM, 30 nM, 40 nM, 50 nM, 60 nM, 70 nM, 80 nM, 90 nM, or 100 nM. In certain embodiments, the first antigen-binding site binds CD19 with a K_{D} of about 10 nM-about 1000 nM, about 10 nM-about 900 nM, about 10 nM-about 800 nM, about 10 nM-about 700 nM, about 10 nM-about 600 nM, about 10 nM-about 500 nM, about 10 nM-about 400 nM, about 10 nM-about 300 nM, about 10 nM-about 200 nM, about 10 nM-about 100 nM, about 10 nM-about 50 nM, about 50 nM-about 1000 nM, about 100 nM-about 1000 nM, about 200 nM-about 1000 nM, about 300 nM-about 1000 nM, about 400 nM-about 1000 nM, about 500 nM-about 1000 nM, about 600 nM-about 1000 nM, about 700 nM-about 1000 nM, about 800 nM-about 1000 nM, or about 900 nM-about 1000 nM.

It is understood that the binding affinity to CD19 of the first antigen-binding site alone may be different from the binding affinity of the same antigen-binding site in the context of the multi-specific binding protein disclosed herein, possibly due to the conformational restraint from the other domains. The context-dependent binding affinity is described in subsection I.G titled “Binding Affinity.”

Melting temperature represents the thermostability of the antigen-binding site and can be measured by differential scanning fluorimetry, for example, as described in Durowoju et al. (2017) J. Vis. Exp. (121): 55262. The thermostability of an antibody or fragment thereof may be enhanced by grafting CDRs onto stable frameworks, introducing non-canonical disulfide bonds, and other mutagenesis, as described in McConnell et al. (2014) MAbs, 6(5): 1274-82; and Goldman et al. (2017) Front. Immunol., 8: 865.

In certain embodiments, the first antigen-binding site has a melting temperature of at least 50° C., at least 55° C., at least 56° C., at least 57° C., at least 58° C., at least 59° C., at least 60° C., at least 61° C., at least 62° C., at least 63° C., at least 64° C., at least 65° C., at least 70° C., at least 75° C., or at least 80° C. In certain embodiments, the first antigen-binding site has a melting temperature in the range of 50-80° C., 50-70° C., 50-65° C., 50-60° C., 50-55° C., 55-70° C., 55-65° C., 55-60° C., 56-65° C., 56-60° C., 57-65° C., 57-60° C., 58-65° C., 58-60° C., 59-65° C., 59-60° C., 60-80° C., 60-75° C., 60-70° C., 60-65° C., 65-80° C., 65-75° C., 65-70° C., 70-80° C., or 70-75° C.

### B. Second Antigen-Binding Site

The second antigen-binding site of the multi-specific binding protein binds CD3 (e.g., human CD3 and/or Macaca CD3). In certain embodiments, the second antigen-binding site binds CD3ε (epsilon). In certain embodiments, the second antigen-binding site binds CD3δ (delta). In certain embodiments, the second antigen-binding site binds CD3γ (gamma).

In certain embodiments, the second antigen-binding site of the multi-specific binding protein binds an epitope at the N-terminus of CD3ε chain. In certain embodiments, the second antigen-binding site binds an epitope localized in amino acid residues 1-27 of human CD3ε extracellular domain. This epitope or a homologous variant thereof is also present in certain non-human primates. Accordingly, in certain embodiments, the second antigen-binding site binds CD3 in different primates, for example, human, new world primates (such as Callithrix jacchus, Saguinus Oedipus, or Saimiri sciureus), old world primates (such as baboons and macaques), gibbons, and non-human homininae. Callithrix jacchus and Saguinus oedipus are new world primates belonging to the family of Callitrichidae, while Saimiri sciureus is a new world primate belonging to the family of Cebidae. In certain embodiments, the second antigen-binding site binds human CD3ε and/or Macaca CD3ε. In certain embodiments, the second antigen-binding site further binds Callithrix jacchus, Saguinus Oedipus, and/or Saimiri sciureus CD3ε.

The second antigen-binding site that binds an extracellular epitope of human and/or Macaca CD3 can be derived from, for example, muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, Fl 11-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, and the antibodies described in WO2008119567A2. For example, the second binding domain optionally can include a VL domain comprising CDR-L1, CDR-L2 and CDR-L3 selected from: (a) CDR-L1 as depicted in SEQ ID NO: 27 of WO2008119567A2, CDR-L2 as depicted in SEQ ID NO: 28 of WO2008119567A2 and CDR-L3 as depicted in SEQ ID NO: 29 of WO2008119567A2; (b) CDR-L1 as depicted in SEQ ID NO: 117 of WO2008119567A2, CDR-L2 as depicted in SEQ ID NO: 118 of WO2008119567A2 and CDR-L3 as depicted in SEQ ID NO: 119 of WO2008119567A2; and (c) CDR-L1 as depicted in SEQ ID NO: 153 of WO2008119567A2, CDR-L2 as depicted in SEQ ID NO: 154 of WO2008119567A2 and CDR-L3 as depicted in SEQ ID NO: 155 of WO2008119567A2. Alternatively, one or more amino acid mutations can be introduced in (a), (b) or (c) group of CDR-L1, CDR-L2 and CDR-L3, and the second binding domain can include any of the mutated groups of CDR-L1, CDR-L2 and CDR-L3.

For example, the second binding domain can include a VL domain comprising CDR-L1, CDR-L2 and CDR-L3 selected from: (a) CDR-H1 as depicted in SEQ ID NO: 12 of WO2008119567A2, CDR-H2 as depicted in SEQ ID NO: 13 of WO2008119567A2 and CDR-H3 as depicted in SEQ ID NO: 14 of WO2008119567A2; (b) CDR-H1 as depicted in SEQ ID NO: 30 of WO2008119567A2, CDR-H2 as depicted in SEQ ID NO: 31 of WO2008119567A2 and CDR-H3 as depicted in SEQ ID NO: 32 of WO2008119567A2; (c) CDR-H1 as depicted in SEQ ID NO: 48 of WO2008119567A2, CDR-H2 as depicted in SEQ ID NO: 49 of WO2008119567A2 and CDR-H3 as depicted in SEQ ID NO: 50 of WO2008119567A2; (d) CDR-H1 as depicted in SEQ ID NO: 66 of WO2008119567A2, CDR-H2 as depicted in SEQ ID NO: 67 of WO2008119567A2 and CDR-H3 as depicted in SEQ ID NO: 68 of WO2008119567A2; (e) CDR-H1 as depicted in SEQ ID NO: 84 of WO2008119567A2, CDR-H2 as depicted in SEQ ID NO: 85 of WO2008119567A2 and CDR-H3 as depicted in SEQ ID NO: 86 of WO2008119567A2; (f) CDR-H1 as depicted in SEQ ID NO: 102 of WO2008119567A2, CDR-H2 as depicted in SEQ ID NO: 103 of WO2008119567A2 and CDR-H3 as depicted in SEQ ID NO: 104 of WO2008119567A2; (g) CDR-H1 as depicted in SEQ ID NO: 120 of WO2008119567A2, CDR-H2 as depicted in SEQ ID NO: 121 of WO2008119567A2 and CDR-H3 as depicted in SEQ ID NO: 122 of WO2008119567A2; (h) CDR-H1 as depicted in SEQ ID NO: 138 of WO2008119567A2, CDR-H2 as depicted in SEQ ID NO: 139 of WO2008119567A2 and CDR-H3 as depicted in SEQ ID NO: 140 of WO2008119567A2; (i) CDR-H1 as depicted in SEQ ID NO: 156 of WO2008119567A2, CDR-H2 as depicted in SEQ ID NO: 157 of WO2008119567A2 and CDR-H3 as depicted in SEQ ID NO: 158 of WO2008119567A2; and (j) CDR-H1 as depicted in SEQ ID NO: 174 of WO2008119567A2, CDR-H2 as depicted in SEQ ID NO: 175 of WO2008119567A2 and CDR-H3 as depicted in SEQ ID NO: 176 of WO2008119567A2. Alternatively, one or more amino acid mutations can be introduced in (a), (b), (c), (d), (e), (f), (g), (h), (i), and (j) group of CDR-H1, CDR-H2 and CDR-H3, and the second binding domain can include any of the mutated groups of CDR-H1, CDR-H2 and CDR-H3. The referenced sequences disclosed in WO2008119567A2 are incorporated by reference herein.

Alternatively, the second domain can be derived from existing CD3 antibodies, for example, muromonab-CD3 (OKT3) as depicted in WO2008101154, otelixizumab (TRX4) as depicted in WO2007145941A2, teplizumab (MGA031) as depicted in WO2013040164A1, visilizumab (Nuvion) as depicted in WO2004052397A1, SP34 as depicted in WO2015181098A1, X35 as depicted in WO2015006749A2, VIT3 as depicted in WO2015006749A2, BMA030 (BW264/56) as depicted in WO2015006749A2, CLB-T3/3 as depicted in WO2004106381A1, CRIS7 as depicted in WO2004106381A1, YTH12.5 as depicted in WO2004106383A1, Fl 11-409 as depicted in WO2012084895A2, CLB-T3.4.2 as depicted in WO2004106381A1, TR-66 as depicted in WO2013158856A2, WT32 as depicted in WO2004106381A1, SPv-T3b as depicted in WO2004106383A1, 11D8 as depicted in WO2004106381A1, XIII-141 as depicted in WO2004106381A1, XIII-46 as depicted in WO 2004106381A1, XIII-87 as depicted in WO2004106381A1, 12F6 as depicted in WO 2004106381A1, T3/RW2-8C8 as depicted in WO2004106381A1, T3/RW2-4B6 as depicted in WO 2004106381A1, OKT3D as depicted in WO2004106381A1, M-T301 as depicted in WO2004106381A1, SMC2 as depicted in WO2004106381A1, F101.01 as depicted in WO2004106381A1, UCHT-1 as depicted in WO2000041474A2 and WT-31 as depicted in WO2016085889A1. The referenced sequences above disclosed in WO2008101154, WO2007145941A2, WO2013040164A1, WO2004052397A1, WO2015181098A1, WO2015006749A2, WO2004106381A1, WO2004106383A1, WO2012084895A2, WO2013158856A2, WO2000041474A2 and WO2016085889A1, are incorporated by reference herein.

A second antigen-binding site that binds CD3 can include a VH comprising three complementarity regions (HCDR1, HCDR2, and HCDR3) and/or a VL comprising three complementarity regions (LCDR1, LCDR2, and LCDR3). Table 2 summarizes, for each variable region, the CDRs of the variable region and scFv constructs based on the given heavy and light chain variable regions. The second antigen-binding site can be derived from the exemplary variable domain and CDR sequences as listed in Table 2.

TABLE 2
Sequences of Exemplary Second Antigen-Binding Sites

| Antigen- | | |
|---|---|---|
| Binding | | |
| Site | VH and HCDRs | VL and LCDRs |
| Adimab | QVQLVQSGAEVKKPGASVKVSCKASGF | DIVMTQSPDSLAVSLGERATINCKS |
| Ab325 | NIKDYYMHWVRQAPGQRLEWMGWIDL | SQSLLNARTGKNYLAWYQQKPGQP |
| | ENANTIYDAKFQGRVTITRDTSASTAYM | PKLLIYWASTRESGVPDRFSGSGSG |
| | ELSSLRSEDTAVYYCARDAYGRYFYDV | TDFTLTISSLQAEDVAVYYCKQSYS |
| | WGQGTLVTVSS (SEQ ID NO: 97) | RRTFGGGTKVEIK (SEQ ID NO: 98) |
| | HCDR1: FNIKDYYMH (SEQ ID NO: 99) | LCDR1: KSSQSLLNARTGKNYLA |
| | HCDR2: WIDLENANTIYDAKFOG (SEQ | (SEQ ID NO: 102) |
| | ID NO: 100) | LCDR2: WASTRES (SEQ ID NO: 103) |
| | HCDR3: ARDAYGRYFYDV (SEQ ID NO: | LCDR3: KQSYSRRT (SEQ ID NO: |
| | 101) | 104) |
| | scFv: | |
| | DIVMTQSPDSLAVSLGERATINCKSSQSLLNARTGKNYLAWYQQKPGQPPKLLIY | |
| | WASTRESGVPDRFSGSGSGTDFTLTISSLQAEDVAVYYCKQSYSRRTFGGGTKVE | |
| | IKGGGGSGGGGSGGGGSGGGGSQVQLVQSGAEVKKPGASVKVSCKASGFNIKD | |
| | YYMHWVRQAPGQRLEWMGWIDLENANTIYDAKFQGRVTITRDTSASTAYMELS | |
| | SLRSEDTAVYYCARDAYGRYFYDVWGQGTLVTVSS (SEQ ID NO: 105) | |
| Harpoon | EVQLVESGGGLVQPGGSLKLSCAASGFT | QTVVTQEPSLTVSPGGTVTLTCASS |
| Ab 2B2 | FNKYAINWVRQAPGKGLEWVARIRSKY | TGAVTSGNYPNWVQQKPGQAPRG |
| | NNYATYYADQVKDRFTISRDDSKNTAY | LIGGTKFLVPGTPARFSGSLLGGKA |
| | LQMNNLKTEDTAVYYCVRHANFGNSYI | ALTLSGVQPEDEAEYYCTLWYSNR |
| | SYWAYWGQGTLVTVSS (SEQ ID NO: | WVFGGGTKLTVL (SEQ ID NO: 107) |
| | 106) | LCDR1: ASSTGAVTSGNYPN (SEQ |
| | HCDR1: GFTFNKYAIN (SEQ ID NO: 108) | ID NO: 111) |
| | HCDR2: RIRSKYNNYATYYADQVK (SEQ | LCDR2: GTKFLVP (SEQ ID NO: 112) |
| | ID NO: 109) | LCDR3: TLWYSNRWV (SEQ ID NO: |
| | HCDR3: HANFGNSYISYWAY (SEQ ID | 113) |
| | NO: 110) | |
| | scFv: | |
| | EVQLVESGGGLVQPGGSLKLSCAASGFTFNKYAINWVRQAPGKGLEWVARIRSK | |
| | YNNYATYYADQVKDRFTISRDDSKNTAYLQMNNLKTEDTAVYYCVRHANFGN | |
| | SYISYWAYWGQGTLVTVSSGGGGSGGGGSGGGGSQTVVTQEPSLTVSPGGTVTL | |
| | TCASSTGAVTSGNYPNWVQQKPGQAPRGLIGGTKFLVPGTPARFSGSLLGGKAA | |
| | LTLSGVQPEDEAEYYCTLWYSNRWVFGGGTKLTVL (SEQ ID NO: 114) | |
| Adimab | QVQLVQSGAEVKKPGASVKVSCKASGF | DIVMTQSPDSLAVSLGERATINCKS |
| mAb393 | NIKDYYMHWVRQAPGQRLEWMGWIDL | SQSLLNSRTGKNYLAWYQQKPGQP |
| | ENANTIYDAKFQGRVTITRDTSASTAYM | PKLLIYWASTRESGVPDRFSGSGSG |
| | ELSSLRSEDTAVYYCARDSYGRYFYDV | TDFTLTISSLQAEDVAVYYCKQSYS |
| | WGQGTLVTVSS (SEQ ID NO: 115) | RRTFGGGTKVEIK (SEQ ID NO: 116) |
| | HCDR1: FNIKDYYMH (SEQ ID NO: 99) | LCDR1: KSSQSLLNARTGKNYLA |
| | HCDR2: WIDLENANTIYDAKFQG (SEQ | (SEQ ID NO: 102) |
| | ID NO: 100) | LCDR2: WASTRES (SEQ ID NO: 103) |
| | HCDR3: ARDSYGRYFYDV (SEQ ID NO: | LCDR3: KQSYSRRT (SEQ ID NO: |
| | 368) | 104) |
| Adimab | QVQLVQSGAEVKKPGASVKVSCKASGF | DIVMTQSPDSLAVSLGERATINCKS |
| mAb333 | NIKDYYMHWVRQAPGQRLEWMGWIDL | SQSLLESRTGKNYLAWYQQKPGQP |
| | ENANTIYDAKFQGRVTITRDTSASTAYM | PKLLIYWASTRESGVPDRFSGSGSG |
| | ELSSLRSEDTAVYYCARDVYGRYFYDL | TDFTLTISSLQAEDVAVYYCKQSYS |
| | WGQGTLVTVSS (SEQ ID NO: 117) | RRTFGGGTKVEIK (SEQ ID NO: 118) |
| | HCDR1: FNIKDYYMH (SEQ ID NO: 99) | LCDR1: KSSQSLLNARTGKNYLA |
| | HCDR2: WIDLENANTIYDAKFOG (SEQ | (SEQ ID NO: 102) |
| | ID NO: 100) | LCDR2: WASTRES (SEQ ID NO: 103) |
| | HCDR3: ARDVYGRYFYDL (SEQ ID NO: | LCDR3: KQSYSRRT (SEQ ID NO: |
| | 369) | 104) |
| Adimab | QVQLVQSGAEVKKPGASVKVSCKASGF | DIVMTQSPDSLAVSLGERATINCKS |
| mAb334 | NIKDYYMHWVRQAPGQRLEWMGWIDL | SQSLLNSRTGKNYLAWYQQKPGQP |
| | ENANTIYDAKFQGRVTITRDTSASTAYM | PKLLIYWASTRESGVPDRFSGSGSG |
| | ELSSLRSEDTAVYYCARDAYGGYFYDV | TDFTLTISSLQAEDVAVYYCKQSYS |
| | WGQGTLVTVSS (SEQ ID NO: 186) | RRTFGCGTKVEIK (SEQ ID NO: 187) |
| | HCDR1: FNIKDYYMH (SEQ ID NO: 99) | LCDR1: KSSQSLLNARTGKNYLA |
| | HCDR2: WIDLENANTIYDAKFOG (SEQ | (SEQ ID NO: 102) |
| | ID NO: 100) | LCDR2: WASTRES (SEQ ID NO: 103) |
| | HCDR3: ARDAYGGYFYDV (SEQ ID NO: | LCDR3: KQSYSRRT (SEQ ID NO: |
| | 370) | 104) |
| Adimab | EVQLLESGGGLVQPGGSLRLSCAASGFT | QTVVTQEPSLSVSPGGTVTLTCGSS |
| mAb404 | FDTYAMNWVRQAPGKGLEWVARIRSK | TGAVTTSNYANWVQQTPGQAPRG |
| | YNNYATYYADSVKDRFTISRDDSKSTLY | LIGGTDKRAPGVPDRFSGSLLGDKA |
| | LQMESLRAEDTAVYYCVRHGNFGNYAV | ALTITGAQAEDEADYYCALWYSNH |
| | SWFAHWGQGTLVTVSS (SEQ ID NO: | WVFGGGTKLTVL (SEQ ID NO: 372) |
| | 371) | LCDR1: GSSTGAVTTSNYAN (SEQ |
| | HCDR1: FTFDTYAMN (SEQ ID NO: 373) | ID NO: 376) |
| | HCDR2: RIRSKYNNYATYYADSVKD | LCDR2: GTDKRAP (SEQ ID NO: 377) |
| | (SEQ ID NO: 374) | LCDR3: ALWYSNHWV (SEQ ID NO: |
| | HCDR3: VRHGNFGNYAVSWFAH (SEQ | 378) |
| | ID NO: 375) | |
| Adimab | EVQLLESGGGLVQPGGSLRLSCAASGFT | QTVVTQEPSLSVSPGGTVTLTCGSS |
| mAb405 | FDTYAMNWVRQAPGKGLEWVARIRSK | TGAVTTSNYANWVQQTPGQAPRG |
| | YNNYATYYADSVKDRFTISRDDSKSTLY | LIGGTDKRAPGVPDRFSGSLLGDKA |
| | LQMESLRAEDTAVYYCVRHGSFGNHIVS | ALTITGAQAEDEADYYCALWYSNH |
| | WFAHWGQGTLVTVSS (SEQ ID NO: 379) | WVFGGGTKLTVL (SEQ ID NO: 372) |
| | HCDR1: FTFDTYAMN (SEQ ID NO: 373) | LCDR1: GSSTGAVTTSNYAN (SEQ |
| | HCDR2: RIRSKYNNYATYYADSVKD | ID NO: 376) |
| | (SEQ ID NO: 374) | LCDR2: GTDKRAP (SEQ ID NO: 377) |
| | HCDR3: VRHGSFGNHIVSWFAH (SEQ ID | LCDR3: ALWYSNHWV (SEQ ID NO: |
| | NO: 399) | 378) |
| Adimab | QVQLVQSGAEVKKPGASVKVSCKASGF | DIVMTQSPDSLAVSLGERATINCKS |
| mAb-1 | NIKDYYMHWVRQAPGQRLEWMGWIDL | SQSLLNSRTRKNYLAWYQQKPGQP |
| | ENGNTIYDAKFQGRVTITRDTSASTAYM | PKLLIYWASTRESGVPDRFSGSGSG |
| | ELSSLRSEDTAVYYCARDGYGRYFYDV | TDFTLTISSLQAEDVAVYYCKQSYS |
| | WGQGTLVTV\\S (SEQ ID NO: 188) | RRTFGGGTKVEIK (SEQ ID NO: 189) |
| Adimab | QVQLVQSGAEVKKPGASVKVSCKASGF | DIVMTQSPDSLAVSLGERATINCKS |
| mAb-2 | NIKDYYMHWVRQAPGQRLEWMGWIDL | SQSLLNNRTRKNYLAWYQQKPGQP |
| | ENANTIYDAKFQGRVTITRDTSASTAYM | PKLLIYWASTRESGVPDRFSGSGSG |
| | ELSSLRSEDTAVYYCARDVYGRYLYDV | TDFTLTISSLQAEDVAVYYCKQSYS |
| | WGQGTLVTVSS (SEQ ID NO: 190) | RRTFGGGTKVEIK (SEQ ID NO: 191) |
| Adimab | QVQLVQSGAEVKKPGASVKVSCKASGF | DIVMTQSPDSLAVSLGERATINCRS |
| mAb-3 | NIKDYYMHWVRQAPGQRLEWMGWIDL | SQSLLNSRTRKNYLAWYQQKPGQP |
| | ENGNTIYDPKFQGRVTITRDTSASTAYM | PKLLIYWASTRESGVPDRFSGSGSG |
| | ELSSLRSEDTAVYYCARDAYGRYFYDV | TDFTLTISSLQAEDVAVYYCKQSYS |
| | WGQGTLVTVSS (SEQ ID NO: 192) | RRTFGGGTKVEIK (SEQ ID NO: 193) |
| Adimab | QVQLVQSGAEVKKPGASVKVSCKASGF | DIVMTQSPDSLAVSLGERATINCKS |
| mAb-4 | NIKDYYMHWVRQAPGQRLEWMGWIDL | SQSLLNGRTRKNYLAWYQQKPGQP |
| | EEGNTIYDAKFQGRVTITRDTSASTAYM | PKLLIYWASTRESGVPDRFSGSGTG |
| | ELSSLRSEDTAVYYCARDAYGRYFYDV | TDFTLTISSLQAEDVAVYYCKQSYS |
| | WGQGTLVTVSS (SEQ ID NO: 194) | RRTFGGGTKVEIK (SEQ ID NO: 195) |
| Adimab | QVQLVQSGAEVKKPGASVKVSCKASGF | DIVMTQSPDSLAVPLGERATINCKS |
| mAb-5 | NIKDYYMHWVRQAPGQRLEWMGWIDL | SQSLLNSRTRKNYLAWYQQKPGQP |
| | ENANTIYDAKFQGRVTITRDTSASTAYM | PKLLIYWASTRESGVPDRFSGSGSG |
| | ELSSLRSEDTAVYYCARDNYGGYFYDV | TDFTLTISSLQAEDVAVYYCKQSYS |
| | WGQGTLVTVSS (SEQ ID NO: 196) | RRTFGGGTKVEIK (SEQ ID NO: 197) |
| Adimab | QVQLVQSGAEVKKPGASVKVSCKASGF | DIVMTQSPDSLAVSLGERATINCKS |
| mAb-6 | NIKDYYMHWVRQAPGQRLEWMGWIDL | SQSLLNSRTRKNYLAWYQQKPGQP |
| | ENGNTIYDPKFQGRVTITRDTSASTAYM | PKLLIYWASTRESGVPDRFSGSGSG |
| | ELSSLRSEDTAVYYCARDGYGRYFYDV | TDFTLTISSLQAEDVAVYYCKQSYS |
| | WGQGTLVTVSS (SEQ ID NO: 198) | LRTFGGGTKVEIK (SEQ ID NO: 199) |
| Adimab | QVQLVQSGAEVKKPGASVKVSCKASGF | DIVMTQSPDSLAVSLGERATINCKS |
| mAb-7 | NIKDYYMHWVRQAPGQRLEWMGWIDL | SQSLLNSRTRKNYLAWYQQKPGQP |
| | ENGNTIYDPKFQGRVTITRDTSASTAYM | PKLLIYWASTRESGVPDRFSGSGSG |
| | ELSSLRSEDTAVYYCARDGYGRYFFDV | TDFTLTISSLQAEDVAVYYCKQSYN |
| | WGQGTLVTVSS (SEQ ID NO: 200) | LRTFGGGTKVEIK (SEQ ID NO: 201) |
| Adimab | QVQLVQSGAEVKKPGASVKVSCKASGF | DIVMTQSPDSLAVSLGERATINCKS |
| mAb-8 | NIKDYYMHWVRQAPGQRLEWMGWIDL | SQSLLNSRTRKNYLAWYQQKPGQP |
| | ENGNTIYDPKFQGRVTITRDTSASTAYM | PKLLIYWASTRESGVPDRFSGSGSG |
| | ELSSLRSEDTAVYYCAREGYGRYFYDV | TDFTLTISSLQAEDVAVYYCKQSYF |
| | WGQGTLVTVSS (SEQ ID NO: 202) | RRAFGGGTKVEIK (SEQ ID NO: 203) |
| Adimab | QVQLVQSGAEVKKPGASVKVSCKASGF | DIVMTQSPDSLAVSLGERATINCKS |
| mAb-9 | NIKDYYMHWVRQAPGQRLEWMGWIDL | SQSLLNSRTRKNYLAWYQQKPGQP |
| | ENGNTIYDPKFQGRVTITRDTSASTAYM | PKLLIYWASTRESGVPDRFSGSGSG |
| | ELSSLRSEDTAVYYCARDGYGRYYYDV | TDFTLTISSLQAEDVAVYYCKQSYN |
| | WGQGTLVTVSS (SEQ ID NO: 204) | LRTFGGGTKLEIK (SEQ ID NO: 205) |
| Adimab | QVQLVQSGAEVKKPGASVKVSCKASGF | DIVMTQSPDSLAVSLGERATINCKS |
| mAb-10 | NIKDYYMHWVRQAPGQRLEWMGWIDL | SQSLLNSRTRKNYLAWYQQKPGQS |
| | ENGNTIYQPKFQGRVTITRDTSASTAYM | PKLLIYWASTRESGVPDRFTGSGSG |
| | ELSSLRSEDTAVYYCARDGYGRYFYDV | TDFTLTISSLQAEDVAVYYCKQSYS |
| | WGQGTLVTVSS (SEQ ID NO: 206) | LRTFGGGTKVEIK (SEQ ID NO: 207) |
| Adimab | QVQLVQSGAEVKKPGASVKVSCKASGF | DIVMTQSPDSLAVSLGERATINCKS |
| mAb-11 | NIKDYYMHWVRQAPGQRLEWMGWIDL | SQSLLESRTGKNYLAWYQQKPGQP |
| | ENGNTIYDPKFQGRVTITRDTSASTAYM | PKLLIYWASTRESGVPDRFSGSGSG |
| | ELSSLRSEDTAVYYCARDGYGRYFYDY | TDFTLTISSLQAEDVAVYYCKQSYS |
| | WGQGTLVTVSS (SEQ ID NO: 208) | LRTFGGGTKVEIK (SEQ ID NO: 209) |
| CD3 | DIKLQQSGAELARPGASVKMSCKTSGYT | VDDIQLTQSPAIMSASPGEKVTMTC |
| binding | FTRYTMHWVKQRPGQGLEWIGYINPSR | RASSSVSYMNWYQQKSGTSPKRWI |
| domain | GYTNYNQKFKDKATLTTDKSSSTAYMQ | YDTSKVASGVPYRFSGSGSGTSYSL |
| in | LSSLTSEDSAVYYCARYYDDHYCLDYW | TISSMEAEDAATYYCQQWSSNPLTF |
| blinatumomab | GQGTTLTVSSVE (SEQ ID NO: 119) | GAGTKLELK (SEQ ID NO: 120) |
| | scFv: | |
| | DIKLQQSGAELARPGASVKMSCKTSGYTFTRYTMHWVKQRPGQGLEWIGYINPS | |
| | RGYTNYNQKFKDKATLTTDKSSSTAYMQLSSLTSEDSAVYYCARYYDDHYCLD | |
| | YWGQGTTLTVSSVEGGSGGSGGSGGSGGVDDIQLTQSPAIMSASPGEKVTMTCR | |
| | ASSSVSYMNWYQQKSGTSPKRWIYDTSKVASGVPYRFSGSGSGTSYSLTISSMEA | |
| | EDAATYYCQQWSSNPLTFGAGTKLELK (SEQ ID NO: 258) | |
| Novimmune | QVQLVESGGGVVQPGRSLRLSCAASGFK | EIVLTQSPATLSLSPGERATLSCRAS |
| 28F11 | FSGYGMHWVRQAPGKGLEWVAVIWYD | QSVSSYLAWYQQKPGQAPRLLIYD |
| | GSKKYYVDSVKGRFTISRDNSKNTLYLQ | ASNRATGIPARFSGSGSGTDFTLTIS |
| | MNSLRAEDTAVYYCARQMGYWHFDLW | SLEPEDFAVYYCQQRSNWPPLTFG |
| | GRGTLVTVSS (SEQ ID NO: 259) | GGTKVEIK (SEQ ID NO: 260) |
| | HCDR1: GYGMH (SEQ ID NO: 261) | LCDR1: RASQSVSSYLA (SEQ ID |
| | HCDR2: VIWYDGSKKYYVDSVKG (SEQ | NO: 264) |
| | ID NO: 262) | LCDR2: DASNRAT (SEQ ID NO: 265) |
| | HCDR3: QMGYWHFDL (SEQ ID NO: 263) | LCDR3: QQRSNWPPLT (SEQ ID NO: |
| | | 266) |
| Novimmune | EVQLLESGGGLVQPGGSLRLSCAASGFT | DFMLTQPHSVSESPGKTVIISCWYQ |
| 27H5 | FSSFPMAWVRQAPGKGLEWVSTISTSGG | QRPGRAPTTVIFGVPDRFSGSIDRSS |
| | RTYYRDSVKGRFTISRDNSKNTLYLQMN | NSASLTISGLQTEDEADYYCFGGGT |
| | SLRAEDTAVYYCAKFRQYSGGFDYWGQ | KLTVLGQPKAAPSVTLFPPSSEELQ |
| | GTLVTVSS (SEQ ID NO: 267) | (SEQ ID NO: 268) |
| Glaxo | EVQLLESGGGLVQPGGSLRLSCAASGFT | DIQLTQPNSVSTSLGSTVKLSCTLSS |
| mAb | FSSFPMAWVRQAPGKGLEWVSTISTSGG | GNIENNYVHWYQLYEGRSPTTMIY |
| | RTYYRDSVKGRFTISRDNSKNTLYLQMN | DDDKRPDGVPDRFSGSIDRSSNSAF |
| | SLRAEDTAVYYCAKFRQYSGGFDYWGQ | LTIHNVAIEDEAIYFCHSYVSSFNVF |
| | GTLVTVSS (SEQ ID NO: 269) | GGGTKLTVLR (SEQ ID NO: 270) |
| Eureka | DVQLVQSGAEVKKPGASVKVSCKASGY | DIVLTQSPATLSLSPGERATLSCRAS |
| mAb | TFTRYTMHWVRQAPGQGLEWIGYINPS | QSVSYMNWYQQKPGKAPKRWIYD |
| | RGYTNYADSVKGRFTITTDKSTSTAYME | TSKVASGVPARFSGSGSGTDYSLTI |
| | LSSLRSEDTATYYCARYYDDHYCLDYW | NSLEAEDAATYYCQQWSSNPLTFG |
| | GQGTTVTVSS (SEQ ID NO: 271) | GGTKVEIK (SEQ ID NO: 272) |
| | scFv: | |
| | DVQLVQSGAEVKKPGASVKVSCKASGYTFTRYTMHWVRQAPGQGLEWIGYINP | |
| | SRGYTNYADSVKGRFTITTDKSTSTAYMELSSLRSEDTATYYCARYYDDHYCLD | |
| | YWGQGTTVTVSSGEGTSTGSGGSGGSGGADDIVLTQSPATLSLSPGERATLSCRA | |
| | SQSVSYMNWYQQKPGKAPKRWIYDTSKVASGVPARFSGSGSGTDYSLTINSLEA | |
| | EDAATYYCQQWSSNPLTFGGGTKVEIK (SEQ ID NO: 273) | |
| Muromonab | QVQLVQSGGGVVQPGRSLRLSCKASGY | DDIQMTQSPSSLSASVGDRVTITCS |
| | TFTRYTMHWVRQAPGKGLEWIGYINPS | ASSSVSYMNWYQQTPGKAPKRWIY |
| | RGYTNYNQKVKDRFTISRDNSKNTAFLQ | DTSKLASGVPSRFSGSGSGTDYTFTI |
| | MDSLRPEDTGVYFCARYYDDHYCLDY | SSLQPEDIATYYCQQWSSNPFTFGQ |
| | WGQGTPVTVSS (SEQ ID NO: 274) | GTKLQIT (SEQ ID NO: 275) |
| MacroGenics | QVQLVQSGGGVVQPGRSLRLSCKASGY | DIQMTQSPSSLSASVGDRVTITCSAS |
| mAb | TFTRYTMHWVRQAPGKGLEWIGYINPS | SSVSYMNWYQQTPGKAPKRWIYD |
| humanized | RGYTNYNQKFKDRFTISTDKSKSTAFLQ | TSKLASGVPSRFSGSGSGTDYTFTIS |
| OKT3 | MDSLRPEDTAVYYCARYYDDHYCLDY | SLQPEDIATYYCQQWSSNPFTFGQG |
| | WGQGTPVTVSS (SEQ ID NO: 276) | TKLQITR (SEQ ID NO: 277) |
| Roche | EVQLLESGGGLVQPGGSLRLSCAASGFT | QAVVTQEPSLTVSPGGTVTLTCGSS |
| CH2527 | FSTYAMNWVRQAPGKGLEWVSRIRSKY | TGAVTTSNYANWVQEKPGQAFRG |
| | NNYATYYADSVKGRFTISRDDSKNTLYL | LIGGTNKRAPGTPARFSGSLLGGKA |
| | QMNSLRAEDTAVYYCVRHGNFGNSYVS | ALTLSGAQPEDEAEYYCALWYSNL |
| | WFAYWGQGTLVTVSS (SEQ ID NO: 278) | WVFGGGTKLTVL (SEQ ID NO: 279) |
| Regeneron | EVQLVESGGGLVQPGRSLRLSCAASGFT | AEIVMTQSPATLSVSPGERATLSCR |
| mAb | FDDYTMHWVRQAPGKGLEWVSGISWN | ASQSVSSNLAWYQQKPGQAPRLLI |
| (anti- | SGSIGYADSVKGRFTISRDNAKKSLYLQ | YGASTRATGIPARFSGSGSGTEFTLT |
| CD3/anti- | MNSLRAEDTALYYCAKDNSGYGHYYY | ISSLQSEDFAVYYCQHYINWPLTFG |
| CD20) | GMDVWGQGTTVTVAS (SEQ ID NO: 280) | GGTKVEIK (SEQ ID NO: 281) |
| WuXi | QVQLVQSGAEVKKPGSSVKVSCKASGY | DIVMTQSPDSLAVSLGERATINCKS |
| WBP3311_ | SFTTYYIHWVRQAPGQGLEWMGWIFPG | SQSLLNSRTRKNYLAWYQQKPGQP |
| 2.166.48- | NDNIKYSEKFKGRVTITADKSTSTAYME | PKLLIYWASTRKSGVPDRFSGSGSG |
| z1 | LSSLRSEDTAVYYCAIDSVSIYYFDYWG | TDFTLTISSLQAEDVAVYYCTQSFIL |
| | QGTLVTVSS (SEQ ID NO: 380) | RTFGGGTKVEIK (SEQ ID NO: 381) |
| | HCDR1: GYSFTTYYIH (SEQ ID NO: 382) | LCDR1: KSSQSLLNSRTRKNYLA |
| | HCDR2: WIFPGNDNIKYSEKFKG (SEQ ID | (SEQ ID NO: 385) |
| | NO: 383) | LCDR2: WASTRKS (SEQ ID NO: 386) |
| | HCDR3: DSVSIYYFDY (SEQ ID NO: 384) | LCDR3: TQSFILRT (SEQ ID NO: 387) |
| WuXi | QVQLVQSGAEVKKPGSSVKVSCKASGF | DIVMTQSPDSLAVSLGERATINCKS |
| WBP3311_ | AFTDYYIHWVRQAPGQGLEWMGWISPG | SQSLLNSRTRKNYLAWYQQKPGQP |
| 2.306.4- | NVNTKYNENFKGRVTITADKSTSTAYM | PKLLIYWASTRQSGVPDRFSGSGSG |
| z1 | ELSSLRSEDTAVYYCARDGYSLYYFDY | TDFTLTISSLQAEDVAVYYCTQSHT |
| | WGQGTLVTVSS (SEQ ID NO: 388) | LRTFGGGTKVEIK (SEQ ID NO: 389) |
| | HCDR1: GFAFTDYYIH (SEQ ID NO: 390) | LCDR1: KSSQSLLNSRTRKNYLA |
| | HCDR2: WISPGNVNTKYNENFKG (SEQ | (SEQ ID NO: 385) |
| | ID NO: 391) | LCDR2: WASTRQS (SEQ ID NO: 393) |
| | HCDR3: DGYSLYYFDY (SEQ ID NO: 392) | LCDR3: TQSHTLRT (SEQ ID NO: |
| | | 394) |
| ADLQ | MAESGGGSVQTGGSLRLSCAYTASSVC | N/A |
| mAb-1 | MAWFRQAPGKEREGVAVTREGLTKTGY | |
| | ADSVKGRFAISQDYAKKTLYLQMSSLKP | |
| | EDTARYYCAARPTSPCTVDGELLASTYN | |
| | YWGQGTQVTV (SEQ ID NO: 395) | |
| ADLQ | MAESGGGSVQTGGSLRLSCAYTASSVC | N/A |
| mAb-2 | MAWFRQAPGKEREGVAVTREGLTKTGY | |
| | ADSVKGRFAISQDYAKKTLYLQMSSLKP | |
| | EDTARYYCAARPTSPCTVDGELLASTYD | |
| | YWGQGTQVTV (SEQ ID NO: 396) | |
| ADLQ | MAESGGGSVQTGGSLRLSCAYTASSVC | N/A |
| mAb-3 | MAWFRQAPGKEREGVAVTREGLTQTGY | |
| | ADSVKGRFAISQDYAKKTLYLQMSSLKP | |
| | EDTARYYCAARPTSPCTVDGELLASTYN | |
| | YWGQGTQVTV (SEQ ID NO: 397) | |
| ADLQ | MAESGGGSVQTGGSLRLSCAYTASSVC | N/A |
| mAb-4 | MAWFRQAPGKEREGVAVTREGLTQTGY | |
| | ADSVKGRFAISQDYAKKTLYLQMSSLKP | |
| | EDTARYYCAARPTSPCTVDGELLASTYD | |
| | YWGQGTQVTV (SEQ ID NO: 398) | |

Where the VL and LCDR sequences are noted as “N/A,” the antigen-binding site is an sdAb having a VH (e.g., VHH) only.

In certain embodiments, the second antigen-binding site comprises a VH comprising complementarity determining regions HCDR1, HCDR2, and HCDR3, and a VL comprising complementarity determining regions LCDR1, LCDR2, and LCDR3. In certain embodiments, the VH comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to the VH of an antibody provided in Table 2, and the VL comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to the VL of the antibody provided in Table 2. In certain embodiments, the VH comprises the HCDR1, HCDR2, and HCDR3 sequences of the antibody provided in Table 2, and the VL comprises the LCDR1, LCDR2, and LCDR3 sequences of the antibody provided in Table 2. In certain embodiments, the VH comprises the amino acid sequence of the VH of an antibody provided in Table 2, and the VL comprises the amino acid sequence of the VL of the antibody provided in Table 2.

In certain embodiments, the VH of the second antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 97, and the VL of the second antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 98. In certain embodiments, the VH comprises HCDR1, HCDR2, and HCDR3 sequences set forth in SEQ ID NOs: 99, 100, 101, respectively, and the VL comprises LCDR1, LCDR2, and LCDR3 sequences set forth in SEQ ID NOs: 102, 103, and 104, respectively. In certain embodiments, the VH comprises the amino acid sequence set forth in SEQ ID NO: 97, and the VL comprises the amino acid sequence set forth in SEQ ID NO: 98.

In certain embodiments, the VH of the second antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 106, and the VL of the second antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 107. In certain embodiments, the VH comprises HCDR1, HCDR2, and HCDR3 sequences set forth in SEQ ID NOs: 108, 109, and 110, respectively, and the VL comprises LCDR1, LCDR2, and LCDR3 sequences set forth in SEQ ID NOs: 111, 112, and 113, respectively. In certain embodiments, the VH comprises the amino acid sequence set forth in SEQ ID NO: 106, and the VL comprises the amino acid sequence set forth in SEQ ID NO: 107.

Such antigen-binding site may take the form of scFv. In certain embodiments, the second antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to an scFv sequence provided in Table 2. In certain embodiments, the second antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 105 or 114.

In other embodiments, the second antigen-binding site comprises an sdAb comprising a VH comprising complementarity determining regions HCDR1, HCDR2, and HCDR3. In certain embodiments, the VH comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to the VH of an sdAb antibody provided in Table 2. In certain embodiments, the VH comprises the HCDR1, HCDR2, and HCDR3 sequences of the antibody provided in Table 2. In certain embodiments, the VH comprises the amino acid sequence of the VH of an sdAb provided in Table 2.

In certain embodiments, the second antigen-binding site competes for binding CD3 (e.g., human CD3 and/or Macaca CD3) with an antibody or antigen-binding fragment thereof comprising the VH, VL and/or scFv sequences provided in Table 2.

In certain embodiments, the second antigen-binding site of the multi-specific binding protein binds CD3 (e.g., human CD3 and/or Macaca CD3) with a dissociation constant (K_{D}) of about 0.1 nM-about 1 μM. The K_{D} can be measured by a method known in the art. In certain embodiments, the K_{D} is measured by SPR to CD3 or an extracellular fragment thereof immobilized on a chip. In certain embodiments, the K_{D} is measured by flow cytometry to CD3 expressed on the surface of cells, for example, following the method described in Example 5 below.

In certain embodiments, the second antigen-binding site binds CD3 with a K_{D} of lower than or equal to 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 90 pM, 80 pM, 70 pM, 60 pM, 50 pM, 40 pM, 30 pM, 20 pM, or 10 pM. For example, in certain embodiments, the first antigen-binding site binds CD3 with a K_{D} of about 10 pM-about 1 nM, about 10 pM-about 0.9 nM, about 10 pM-about 0.8 nM, about 10 pM-about 0.7 nM, about 10 pM-about 0.6 nM, about 10 pM nM-about 0.5 nM, about 10 pM-about 0.4 nM, about 10 pM-about 0.3 nM, about 10 pM-about 0.2 nM, about 10 pM-about 0.1 nM, about 10 pM-about 50 pM, 0.1 nM-about 10 nM, about 0.1 nM-about 9 nM, about 0.1 nM-about 8 nM, about 0.1 nM-about 7 nM, about 0.1 nM-about 6 nM, about 0.1 nM-about 5 nM, about 0.1 nM-about 4 nM, about 0.1 nM-about 3 nM, about 0.1 nM-about 2 nM, about 0.1 nM-about 1 nM, about 0.1 nM-about 0.5 nM, about 0.5 nM-about 10 nM, about 1 nM-about 10 nM, about 2 nM-about 10 nM, about 3 nM-about 10 nM, about 4 nM-about 10 nM, about 5 nM-about 10 nM, about 6 nM-about 10 nM, about 7 nM-about 10 nM, about 8 nM-about 10 nM, or about 9 nM-about 10 nM.

In certain embodiments, the second antigen-binding site binds CD3 with a K_{D} greater than or equal to 10 nM, 20 nM, 30 nM, 40 nM, 50 nM, 60 nM, 70 nM, 80 nM, 90 nM, or 100 nM. In certain embodiments, the second antigen-binding site binds CD3 with a K_{D} of about 10 nM-about 1000 nM, about 10 nM-about 900 nM, about 10 nM-about 800 nM, about 10 nM-about 700 nM, about 10 nM-about 600 nM, about 10 nM-about 500 nM, about 10 nM-about 400 nM, about 10 nM-about 300 nM, about 10 nM-about 200 nM, about 10 nM-about 100 nM, about 10 nM-about 50 nM, about 50 nM-about 1000 nM, about 100 nM-about 1000 nM, about 200 nM-about 1000 nM, about 300 nM-about 1000 nM, about 400 nM-about 1000 nM, about 500 nM-about 1000 nM, about 600 nM-about 1000 nM, about 700 nM-about 1000 nM, about 800 nM-about 1000 nM, or about 900 nM-about 1000 nM.

It is understood that the binding affinity to CD3 of the second antigen-binding site alone may be different from the binding affinity of the same antigen-binding site in the context of the multi-specific binding protein disclosed herein, possibly due to the conformational restraint from the other domains. The context-dependent binding affinity is described in the subsection I.G titled “Binding Affinity.”

In certain embodiments, the second antigen-binding site has a melting temperature of at least 50° C., at least 55° C., at least 56° C., at least 57° C., at least 58° C., at least 59° C., at least 60° C., at least 61° C., at least 62° C., at least 63° C., at least 64° C., at least 65° C., at least 70° C., at least 75° C., or at least 80° C. In certain embodiments, the second antigen-binding site has a melting temperature in the range of 50-80° C., 50-70° C., 50-65° C., 50-60° C., 50-55° C., 55-70° C., 55-65° C., 55-60° C., 56-65° C., 56-60° C., 57-65° C., 57-60° C., 58-65° C., 58-60° C., 59-65° C., 59-60° C., 60-80° C., 60-75° C., 60-70° C., 60-65° C., 65-80° C., 65-75° C., 65-70° C., 70-80° C., or 70-75° C.

### C. Third Antigen-Binding Site

The third antigen-binding site of the multi-specific binding protein binds serum albumin (e.g., HSA). In certain embodiments, the third antigen-binding site has a higher binding affinity to a human serum albumin than to a mouse serum albumin. In certain embodiments, the third antigen-binding site does not bind the D-III domain of HSA. In certain embodiments, the third antigen-binding site extends the serum half-life of the multi-specific binding protein.

The third antigen-binding site that binds serum albumin can be derived from, for example, the antigen-binding sites disclosed in U.S. Pat. No. 8,188,223, and PCT Publication Nos. WO2017085172, and WO2018050833.

A third antigen-binding site that binds HSA can include a VH comprising three complementarity regions (HCDR1, HCDR2, and HCDR3) and/or a VL comprising three complementarity regions (LCDR1, LCDR2, and LCDR3). Table 3 summarizes, for each variable region, the CDRs of the variable region and scFv constructs based on the given heavy and light chain variable regions. The third antigen-binding site can be derived from the exemplary variable domain and CDR sequences as listed in Table 3.

TABLE 3
Sequences of Exemplary Third Antigen-Binding Sites

| Antigen- | | |
|---|---|---|
| Binding | | |
| Site | VH and HCDRs | VL and LCDRs |
| Ablynx | EVQLVESGGGLVQPGNSLRLSCAASGFT | N/A |
| ALB8 | FSSFGMSWVRQAPGKGLEWVSSISGSGS | |
| | DTLYADSVKGRFTISRDNAKTTLYLQMN | |
| | SLRPEDTAVYYCTIGGSLSRSSQGTLVTV | |
| | SS (SEQ ID NO: 121) | |
| | HCDR1: SFGMS (SEQ ID NO: 122) or | |
| | GFTFSSFGMS (SEQ ID NO: 123) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGLVQPGGSLRLSCAASGFT | N/A |
| ALB3 | FRSFGMSWVRQAPGKEPEWVSSISGSGS | |
| | DTLYADSVKGRFTISRDNAKTTLYLQMN | |
| | SLKPEDTAVYYCTIGGSLSRSSQGTQVT | |
| | VSS (SEQ ID NO: 127) | |
| | HCDR1: SFGMS (SEQ ID NO: 122) or | |
| | GFTFRSFGMS (SEQ ID NO: 128) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGLVQPGGSLRLSCAASGFT | N/A |
| ALB4 | FSSFGMSWVRQAPGKEPEWVSSISGSGS | |
| | DTLYADSVKGRFTISRDNAKTTLYLQMN | |
| | SLKPEDTAVYYCTIGGSLSRSSQGTQVT | |
| | VSS (SEQ ID NO: 129) | |
| | HCDR1: SFGMS (SEQ ID NO: 122) or | |
| | GFTFSSFGMS (SEQ ID NO: 123) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGLVQPGGSLRLSCAASGFT | N/A |
| ALB5 | FRSFGMSWVRQAPGKGLEWVSSISGSGS | |
| | DTLYADSVKGRFTISRDNAKTTLYLQMN | |
| | SLKPEDTAVYYCTIGGSLSRSSQGTQVT | |
| | VSS (SEQ ID NO: 130) | |
| | HCDR1: SFGMS (SEQ ID NO: 122) or | |
| | GFTFRSFGMS (SEQ ID NO: 128) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGLVQPGNSLRLSCAASGFT | N/A |
| ALB6 | FRSFGMSWVRQAPGKGLEWVSSISGSGS | |
| | DTLYADSVKGRFTISRDNAKTTLYLQMN | |
| | SLKPEDTAVYYCTIGGSLSRSSQGTLVTV | |
| | SS (SEQ ID NO: 131) | |
| | HCDR1: SFGMS (SEQ ID NO: 122) or | |
| | GFTFRSFGMS (SEQ ID NO: 128) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGLVQPGNSLRLSCAASGFT | N/A |
| ALB7 | FRSFGMSWVRQAPGKGLEWVSSISGSGS | |
| | DTLYADSVKGRFTISRDNAKTTLYLQMN | |
| | SLRPEDTAVYYCTIGGSLSRSSQGTLVTV | |
| | SS (SEQ ID NO: 132) | |
| | HCDR1: SFGMS (SEQ ID NO: 122) or | |
| | GFTFRSFGMS (SEQ ID NO: 128) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGLVQPGNSLRLSCAASGFT | N/A |
| ALB9 | FSSFGMSWVRQAPGKGLEWVSSISGSGS | |
| | DTLYADSVKGRFTISRDNAKNTLYLQM | |
| | NSLRPEDTAVYYCTIGGSLSRSSQGTLVT | |
| | VSS (SEQ ID NO: 133) | |
| | HCDR1: SFGMS (SEQ ID NO: 122) or | |
| | GFTFSSFGMS (SEQ ID NO: 123) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGLVQPGNSLRLSCAASGFT | N/A |
| ALB10 | FSSFGMSWVRQAPGKGLEWVSSISGSGS | |
| | DTLYADSVKGRFTISRDNAKNTLYLQM | |
| | NSLRPEDTAVYYCTIGGSLSRSGQGTLV | |
| | TVSS (SEQ ID NO: 134) | |
| | HCDR1: SFGMS (SEQ ID NO: 122) or | |
| | GFTFSSFGMS (SEQ ID NO: 123) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLLESGGGLVQPGGSLRLSCAASGFT | N/A |
| ALB23 | FRSFGMSWVRQAPGKGPEWVSSISGSGS | |
| | DTLYADSVKGRFTISRDNSKNTLYLQMN | |
| | SLRPEDTAVYYCTIGGSLSRSSQGTLVTV | |
| | SS (SEQ ID NO: 135) | |
| | HCDR1: SFGMS (SEQ ID NO: 122) or | |
| | GFTFRSFGMS (SEQ ID NO: 128) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGVVQPGNSLRLSCAASGFT | N/A |
| mAb-1 | FSSFGMSWVRQAPGKGLEWVSSISGSGS | |
| | DTLYADSVKGRFTISRDNAKTTLYLQMN | |
| | SLRPEDTATYYCTIGGSLSRSSQGTLVTV | |
| | SSA (SEQ ID NO: 167) | |
| | HCDR1: SFGMS (SEQ ID NO: 122) or | |
| | GFTFSSFGMS (SEQ ID NO: 123) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGVVQPGGSLRLSCAASGFT | N/A |
| mAb-2 | FRSFGMSWVRQAPGKGPEWVSSISGSGS | |
| | DTLYADSVKGRFTISRDNSKNTLYLQMN | |
| | SLRPEDTATYYCTIGGSLSRSSQGTLVTV | |
| | SSA (SEQ ID NO: 168) | |
| | HCDR1: SFGMS (SEQ ID NO: 122) or | |
| | GFTFRSFGMS (SEQ ID NO: 128) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGLVQPGNSLRLSCAASGFT | N/A |
| mAb-3 | FSSFGMSWVRQAPGKGLEWVSSISGSGS | |
| | DTLYADSVKGRFTISRDNAKTTLYLQMN | |
| | SLRPEDTAVYYCTIGGSLSRSSQGTLVKV | |
| | SSA (SEQ ID NO: 169) | |
| | HCDR1: SFGMS (SEQ ID NO: 122) or | |
| | GFTFSSFGMS (SEQ ID NO: 123) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGVVQPGNSLRLSCAASGFT | N/A |
| mAb-4 | FSSFGMSWVRQAPGKGLEWVSSISGSGS | |
| | DTLYADSVKGRFTISRDNAKTTLYLQMN | |
| | SLRPEDTALYYCTIGGSLSRSSQGTLVTV | |
| | SS (SEQ ID NO: 170) | |
| | HCDR1: SFGMS (SEQ ID NO: 122) or | |
| | GFTFSSFGMS (SEQ ID NO: 123) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGVVQPGNSLRLSCAASGFT | N/A |
| mAb-5 | FSSFGMSWVRQAPGKGLEWVSSISGSGS | |
| | DTLYADSVKGRFTISRDNAKTTLYLQMN | |
| | SLRPEDTALYYCTIGGSLSRSSQGTLVTV | |
| | SSA (SEQ ID NO: 171) | |
| | HCDR1: SFGMS (SEQ ID NO: 122) or | |
| | GFTFSSFGMS (SEQ ID NO: 123) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGVVQPGNSLRLSCAASGFT | N/A |
| mAb-6 | FSSFGMSWVRQAPGKGLEWVSSISGSGS | |
| | DTLYADSVKGRFTISRDNAKTTLYLQMN | |
| | SLRPEDTALYYCTIGGSLSRSSQGTLVTV | |
| | SSAA (SEQ ID NO: 172) | |
| | HCDR1: SFGMS (SEQ ID NO: 122) or | |
| | GFTFSSFGMS (SEQ ID NO: 123) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGVVQPGNSLRLSCAASGFT | N/A |
| mAb-7 | FSSFGMSWVRQAPGKGLEWVSSISGSGS | |
| | DTLYADSVKGRFTISRDNAKTTLYLQMN | |
| | SLRPEDTALYYCTIGGSLSRSSQGTLVTV | |
| | SSAAA (SEQ ID NO: 173) | |
| | HCDR1: SFGMS (SEQ ID NO: 122) or | |
| | GFTFSSFGMS (SEQ ID NO: 123) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGVVQPGNSLRLSCAASGFT | N/A |
| mAb-8 | FSSFGMSWVRQAPGKGLEWVSSISGSGS | |
| | DTLYADSVKGRFTISRDNAKTTLYLQMN | |
| | SLRPEDTALYYCTIGGSLSRSSQGTLVTV | |
| | SSG (SEQ ID NO: 174) | |
| | HCDR1: SFGMS (SEQ ID NO: 122) or | |
| | GFTFSSFGMS (SEQ ID NO: 123) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGVVQPGNSLRLSCAASGFT | N/A |
| mAb-9 | FSSFGMSWVRQAPGKGLEWVSSISGSGS | |
| | DTLYADSVKGRFTISRDNAKTTLYLQMN | |
| | SLRPEDTALYYCTIGGSLSRSSQGTLVTV | |
| | SSGG (SEQ ID NO: 175) | |
| | HCDR1: SFGMS (SEQ ID NO: 122) or | |
| | GFTFSSFGMS (SEQ ID NO: 123) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | EVQLVESGGGVVQPGNSLRLSCAASGFT | N/A |
| mAb-10 | FSSFGMSWVRQAPGKGLEWVSSISGSGS | |
| | DTLYADSVKGRFTISRDNAKTTLYLQMN | |
| | SLRPEDTALYYCTIGGSLSRSSQGTLVTV | |
| | SSGGG (SEQ ID NO: 176) | |
| | HCDR1: SFGMS (SEQ ID NO: 122) or | |
| | GFTFSSFGMS (SEQ ID NO: 123) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | AVQLVESGGGLVQPGNSLRLSCAASGFT | N/A |
| PMP6A6 | FRSFGMSWVRQAPGKEPEWVSSISGSGS | |
| | DTLYADSVKGRFTISRDNAKTTLYLQMN | |
| | SLKPEDTAVYYCTIGGSLSRSSQGTQVT | |
| | VSS (SEQ ID NO: 136) | |
| | HCDR1: SFGMS (SEQ ID NO: 122) or | |
| | GFTFRSFGMS (SEQ ID NO: 128) | |
| | HCDR2: SISGSGSDTL (SEQ ID NO: 124) | |
| | or SISGSGSDTLYADSVKG (SEQ ID NO: | |
| | 125) | |
| | HCDR3: GGSLSR (SEQ ID NO: 126) | |
| Ablynx | AVQLVDSGGGLVQPGGSLRLSCAASGFS | N/A |
| PMP6C1 | FGSFGMSWVRQYPGKEPEWVSSINGRG | |
| | DDTRYADSVKGRFSISRDNAKNTLYLQ | |
| | MNSLKPEDTAEYYCTIGRSVSRSRTQGT | |
| | QVTVSS (SEQ ID NO: 137) | |
| | HCDR1: SFGMS (SEQ ID NO: 122) or | |
| | GFSFGSFGMS (SEQ ID NO: 138) | |
| | HCDR2: SINGRGDDTR (SEQ ID NO: 139) | |
| | or SINGRGDDTRYADSVKG (SEQ ID NO: | |
| | 140) | |
| | HCDR3: GRSVSRS (SEQ ID NO: 141) | |
| Ablynx | AVQLVESGGGLVQPGGSLRLTCTASGFT | N/A |
| PMP6G8 | FRSFGMSWVRQAPGKDQEWVSAISADS | |
| | STKNYADSVKGRFTISRDNAKKMLYLE | |
| | MNSLKPEDTA VYYCVIGRGSPSSPGTQV | |
| | TVSS (SEQ ID NO: 142) | |
| | HCDR1: SFGMS (SEQ ID NO: 122) or | |
| | GFTFRSFGMS (SEQ ID NO: 128) | |
| | HCDR2: AISADSSTKN (SEQ ID NO: 143) | |
| | or AISADSSTKNYADSVKG (SEQ ID NO: | |
| | 144) | |
| | HCDR3: GRGSP(SEQ ID NO: 145) | |
| Ablynx | QVQLAESGGGLVQPGGSLRLTCTASGFT | N/A |
| PMP6A5 | FGSFGMSWVRQAPGEGLEWVSAISADSS | |
| | DKRYADSVKGRFTISRDNAKKMLYLEM | |
| | NSLKSEDTAVYYCVIGRGSPASQGTQVT | |
| | VSS (SEQ ID NO: 146) | |
| | HCDR1: SFGMS (SEQ ID NO: 122) or | |
| | GFTFGSFGMS (SEQ ID NO: 147) | |
| | HCDR2: AISADSSDKR (SEQ ID NO: 148) | |
| | or AISADSSDKRYADSVKG (SEQ ID NO: | |
| | 149) | |
| | HCDR3: GRGSP(SEQ ID NO: 145) | |
| Ablynx | QVQLVESGGGLVQPGGSLRLSCAASGFT | N/A |
| PMP6G7 | FSNYWMYWVRVAPGKGLERISRDISTG | |
| | GGYSYYADSVKGRFTISRDNAKNTLYLQ | |
| | MNSLKPEDTALYYCAKDREAQVDTLDF | |
| | DYRGQGTQVTVSS (SEQ ID NO: 150) | |
| | HCDR1: NYWMY (SEQ ID NO: 151) or | |
| | GFTFSNYWMY (SEQ ID NO: 152) | |
| | HCDR2: RDISTGGGYSY (SEQ ID NO: | |
| | 153) or RDISTGGGYSYYADSVKG (SEQ | |
| | ID NO: 154) | |
| | HCDR3: DREAQVDTLDFDY (SEQ ID NO: | |
| | 155) | |
| Ablynx | AVQLVESGGGLVQGGGSLRLACAASERI | N/A |
| PMP6A8 | FDLNLMGWYRQGPGNERELVATCITVG | |
| | DSTNYADSVKGRFTISMDYTKQTVYLH | |
| | MNSLRPEDTGLYYCKIRRTWHSELWGQ | |
| | GTQVTVSS (SEQ ID NO: 156) | |
| | HCDR1: LNLMG (SEQ ID NO: 157) or | |
| | SERIFDLNLMG (SEQ ID NO: 158) | |
| | HCDR2: TCITVGDSTN (SEQ ID NO: 159) | |
| | or TCITVGDSTNYADSVKG (SEQ ID NO: | |
| | 160) | |
| | HCDR3: RRTWHSEL (SEQ ID NO: 161) | |
| Ablynx | EVQLVESGGGLVQEGGSLRLACAASERI | N/A |
| PMP6C1 | WDINLLGWYRQGPGNERELVATITVGD | |
| | STSYADSVKGRFTISRDYDKNTLYLQMN | |
| | SLRPEDTGLYYCKIRRTWHSELWGQGT | |
| | QVTVSS (SEQ ID NO: 162) | |
| | HCDR1: INLLG (SEQ ID NO: 163) or | |
| | SERIWDINLLG (SEQ ID NO: 164) | |
| | HCDR2: TITVGDSTS (SEQ ID NO: 165) or | |
| | TITVGDSTSYADSVKG (SEQ ID NO: 166) | |
| | HCDR3: RRTWHSEL (SEQ ID NO: 161) | |
| Harpoon | EVQLVESGGGLVQPGNSLRLSCAASGFT | N/A |
| mAb-1 | FSKFGMSWVRQAPGKGLEWVSSISGSGR | |
| | DTLYAESVKGRFTISRDNAKTTLYLQMN | |
| | SLRPEDTAVYYCTIGGSLSVSSQGTLVTV | |
| | SS (SEQ ID NO: 177) | |
| | HCDR1: GFTFSKFGMS (SEQ ID NO: 178) | |
| | HCDR2: SISGSGRDTLYAESVK (SEQ ID | |
| | NO: 179) | |
| | HCDR3: GGSLSV (SEQ ID NO: 180) | |
| Harpoon | EVQLVESGGGLVQPGNSLRLSCAASGFT | N/A |
| mAb-2 | FSRFGMSWVRQAPGKGLEWVSSISGSGS | |
| | DTLYAESVKGRFTISRDNAKTTLYLQMN | |
| | SLRPEDTAVYYCTIGGSLSRSSQGTLVTV | |
| | SS (SEQ ID NO: 181) | |
| | HCDR1: GFTFSRFGMS (SEQ ID NO: 182) | |
| | HCDR2: SISGSGSDTLYAESVK (SEQ ID | |
| | NO: 183) | |
| | HCDR3: GGSLSR (SEQ ID NO: 126) | |
| Harpoon | EVQLVESGGGLVQPGNSLRLSCAASGFT | N/A |
| mAb-3 | FSKFGMSWVRQAPGKGLEWVSSISGSGT | |
| | DTLYAESVKGRFTISRDNAKTTLYLQMN | |
| | SLRPEDTAVYYCTIGGSLSRSSQGTLVTV | |
| | SS (SEQ ID NO: 184) | |
| | HCDR1: GFTFSKFGMS (SEQ ID NO: 178) | |
| | HCDR2: SISGSGTDTLYAESVK (SEQ ID | |
| | NO: 185) | |
| | HCDR3: GGSLSR (SEQ ID NO: 126) | |
| Domantis | EVQLLESGGGLVQPGGSLRLSCAASGFT | N/A |
| DOM7h- | FSKYWMSWVRQAPGKGLEWVSSIDFM | |
| 22 | GPHTYYADSVKGRFTISRDNSKNTLYLQ | |
| | MNSLRAEDTAVYYCAKGRTSMLPMKG | |
| | KFDYWGQGTLVTVSS (SEQ ID NO: 283) | |
| Domantis | EVQLLESGGGLVQPGGSLRLSCTASGFT | |
| DOM7h- | FDEYNMSWVRQAPGKGLEWVSTILPHG | |
| 26 | DRTYYADSVKGRFTISRDNSKNTLYLQM | |
| | NSLRAEDTAVYYCAKQDPLYRFDYWGQ | |
| | GTLVTVSS (SEQ ID NO: 284) | |
| Domantis | N/A | DIQMTQSPSSLSASVGDRVTITCRAS |
| DOM7h- | | QKIATYLNWYQQKPGKAPKLLIYR |
| 2 | | SSSLQSAVPSRFSGSGSGTVFTLTISS |
| | | LQPEDFATYYCQQTYAVPPTFGQG |
| | | TKVEIKR (SEQ ID NO: 285) |
| Domantis | N/A | DIQMTQSPSSLSASVGDRVTITCRAS |
| DOM7h- | | QSISSYLNWYQQKPGKAPKLLIYRN |
| 8 | | SPLQSGVPSRFSGSGSGTDFTLTISSL |
| | | QPEDFATYYCQQTYRVPPTFGQGT |
| | | KVEIKR (SEQ ID NO: 286) |
| MSA21 | QVQLQESGGGLVQPGGSLRLSCEASGFT | N/A |
| | FSRFGMTWVRQAPGKGVEWVSGISSLG | |
| | DSTLYADSVKGRFTISRDNAKNTLYLQM | |
| | NSLKPEDTAVYYCTIGGSLNPGGQGTQV | |
| | TVSS (SEQ ID NO: 287) | |
| UCB | EVQLLESGGGLVQPGGSLRLSCAVSGID | DIQMTQSPSSVSASVGDRVTITCQSS |
| mAb-1 | LSNYAINWVRQAPGKCLEWIGIIWASGT | PSVWSNFLSWYQQKPGKAPKLLIY |
| | TFYATWAKGRFTISRDNSKNTVYLQMN | EASKLTSGVPSRFSGSGSGTDFTLTI |
| | SLRAEDTAVYYCARTVPGYSTAPYFDL | SSLQPEDFATYYCGGGYSSISDTTF |
| | WGQGTLVTVSS (SEQ ID NO: 288) | GCGTKVEIKRT (SEQ ID NO: 289) |
| UCB | EVQLLESGGGLVQPGGSLRLSCAVSGID | DIQMTQSPSSVSASVGDRVTITCQSS |
| mAb-2 | LSNYAINWVRQAPGKGLEWIGIIWASGT | PSVWSNFLSWYQQKPGKAPKLLIY |
| | TFYATWAKGRFTISRDNSKNTVYLQMN | EASKLTSGVPSRFSGSGSGTDFTLTI |
| | SLRAEDTAVYYCARTVPGYSTAPYFDL | SSLQPEDFATYYCGGGYSSISDTTF |
| | WGQGTLVTVSS (SEQ ID NO: 290) | GGGTKVEIKRT (SEQ ID NO: 291) |

Where the VL and LCDR sequences are noted as “N/A,” the antigen-binding site is an sdAb having a VH (e.g., VHH) only. Where the VH and HCDR sequences are noted as “N/A,” the antigen-binding site is an sdAb having a VL only.

In certain embodiments, the third antigen-binding site comprises a VH comprising complementarity determining regions HCDR1, HCDR2, and HCDR3, and a VL comprising complementarity determining regions LCDR1, LCDR2, and LCDR3. In certain embodiments, the VH comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to the VH of an antibody provided in Table 3, and the VL comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to the VL of the antibody provided in Table 3. In certain embodiments, the VH comprises the HCDR1, HCDR2, and HCDR3 sequences of the antibody provided in Table 3, and the VL comprises the LCDR1, LCDR2, and LCDR3 sequences of the antibody provided in Table 3. In certain embodiments, the VH comprises the amino acid sequence of the VH of an antibody provided in Table 3, and the VL comprises the amino acid sequence of the VL of the antibody provided in Table 3.

In other embodiments, the third antigen-binding site comprises an sdAb comprising a VH comprising complementarity determining regions HCDR1, HCDR2, and HCDR3. In certain embodiments, the VH comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to the VH of an sdAb antibody provided in Table 3. In certain embodiments, the VH comprises the HCDR1, HCDR2, and HCDR3 sequences of the antibody provided in Table 3. In certain embodiments, the VH comprises the amino acid sequence of the VH of an sdAb provided in Table 3.

In certain embodiments, the VH of the third antigen-binding site comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, or at least 99%) identical to SEQ ID NO: 121. In certain embodiments, the VH comprises HCDR1, HCDR2, and HCDR3 sequences set forth in SEQ ID NOs: 122 or 123, 124 or 125, and 126, respectively. In certain embodiments, the VH comprises HCDR1, HCDR2, and HCDR3 sequences set forth in SEQ ID NOs: 122, 125, and 126, respectively. In certain embodiments, the VH comprises HCDR1, HCDR2, and HCDR3 sequences set forth in SEQ ID NOs: 123, 124, and 126, respectively. In certain embodiments, the VH comprises the amino acid sequence set forth in SEQ ID NO: 121.

In certain embodiments, the third antigen-binding site competes for binding serum albumin (e.g., HSA) with an antibody or antigen-binding fragment thereof comprising the VH, VL and/or scFv sequences provided in Table 3.

In certain embodiments, the third antigen-binding site of the multi-specific binding protein binds serum albumin (e.g., HSA) with a dissociation constant (K_{D}) of about 0.1 nM-about 100 μM. The K_{D} can be measured by a method known in the art. In certain embodiments, the K_{D} is measured by SPR to serum albumin or a fragment thereof immobilized on a chip.

In certain embodiments, the third antigen-binding site binds serum albumin with a K_{D} of lower than or equal to 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 90 pM, 80 pM, 70 pM, 60 pM, 50 pM, 40 pM, 30 pM, 20 pM, or 10 pM. For example, in certain embodiments, the third antigen-binding site binds serum albumin with a K_{D} of about 10 pM-about 1 nM, about 10 pM-about 0.9 nM, about 10 pM-about 0.8 nM, about 10 pM-about 0.7 nM, about 10 pM-about 0.6 nM, about 10 pM nM-about 0.5 nM, about 10 pM-about 0.4 nM, about 10 pM-about 0.3 nM, about 10 pM-about 0.2 nM, about 10 pM-about 0.1 nM, about 10 pM-about 50 pM, 0.1 nM-about 10 nM, about 0.1 nM-about 9 nM, about 0.1 nM-about 8 nM, about 0.1 nM-about 7 nM, about 0.1 nM-about 6 nM, about 0.1 nM-about 5 nM, about 0.1 nM-about 4 nM, about 0.1 nM-about 3 nM, about 0.1 nM-about 2 nM, about 0.1 nM-about 1 nM, about 0.1 nM-about 0.5 nM, about 0.5 nM-about 10 nM, about 1 nM-about 10 nM, about 2 nM-about 10 nM, about 3 nM-about 10 nM, about 4 nM-about 10 nM, about 5 nM-about 10 nM, about 6 nM-about 10 nM, about 7 nM-about 10 nM, about 8 nM-about 10 nM, about 9 nM-about 10 nM, about 1 nM-about 15 nM, about 2 nM-about 15 nM, about 3 nM-about 15 nM, about 4 nM-about 15 nM, about 5 nM-about 15 nM, about 6 nM-about 15 nM, about 7 nM-about 15 nM, about 8 nM-about 15 nM, about 9 nM-about 15 nM, about 10 nM-about 15 nM, about 11 nM-about 15 nM, about 12 nM-about 15 nM, about 13 nM-about 15 nM, about 14 nM-about 15 nM, about 1 nM-about 20 nM, about 2 nM-about 20 nM, about 3 nM-about 20 nM, about 4 nM-about 20 nM, about 5 nM-about 20 nM, about 6 nM-about 20 nM, about 7 nM-about 20 nM, about 8 nM-about 20 nM, about 9 nM-about 20 nM, about 10 nM-about 20 nM, about 11 nM-about 20 nM, about 12 nM-about 20 nM, about 13 nM-about 20 nM, about 14 nM-about 20 nM, about 15 nM-about 20 nM, about 16 nM-about 20 nM, about 17 nM-about 20 nM, about 18 nM-about 20 nM, or about 19 nM-about 20 nM.

In certain embodiments, the third antigen-binding site binds serum albumin with a K_{D} greater than or equal to 10 nM, 20 nM, 30 nM, 40 nM, 50 nM, 60 nM, 70 nM, 80 nM, 90 nM, or 100 nM. In certain embodiments, the third antigen-binding site binds serum albumin with a K_{D} of about 10 nM-about 1000 nM, about 10 nM-about 900 nM, about 10 nM-about 800 nM, about 10 nM-about 700 nM, about 10 nM-about 600 nM, about 10 nM-about 500 nM, about 10 nM-about 400 nM, about 10 nM-about 300 nM, about 10 nM-about 200 nM, about 10 nM-about 100 nM, about 10 nM-about 50 nM, about 50 nM-about 1000 nM, about 100 nM-about 1000 nM, about 200 nM-about 1000 nM, about 300 nM-about 1000 nM, about 400 nM-about 1000 nM, about 500 nM-about 1000 nM, about 600 nM-about 1000 nM, about 700 nM-about 1000 nM, about 800 nM-about 1000 nM, or about 900 nM-about 1000 nM.

It is understood that the binding affinity to serum albumin of the third antigen-binding site alone may be different from the binding affinity of the same antigen-binding site in the context of the multi-specific binding protein disclosed herein, possibly due to the conformational restraint from the other domains. The context-dependent binding affinity is described in subsection I.G titled “Binding Affinity.”

In certain embodiments, the third antigen-binding site has a melting temperature of at least 50° C., at least 55° C., at least 56° C., at least 57° C., at least 58° C., at least 59° C., at least 60° C., at least 61° C., at least 62° C., at least 63° C., at least 64° C., at least 65° C., at least 70° C., at least 75° C., or at least 80° C. In certain embodiments, the third antigen-binding site has a melting temperature in the range of 50-80° C., 50-70° C., 50-65° C., 50-60° C., 50-55° C., 55-70° C., 55-65° C., 55-60° C., 56-65° C., 56-60° C., 57-65° C., 57-60° C., 58-65° C., 58-60° C., 59-65° C., 59-60° C., 60-80° C., 60-75° C., 60-70° C., 60-65° C., 65-80° C., 65-75° C., 65-70° C., 70-80° C., or 70-75° C.

### D. Humanization and Deimmunization

The first, second, and/or third antigen-binding sites of the multi-specific binding protein disclosed herein may be humanized, for example, from one or more antigen-binding sites disclosed above, to optimize the immunogenicity and binding properties of the multi-specific binding protein, thereby enhancing the therapeutic index of the multi-specific binding protein.

“Humanized” antibodies or fragments thereof (e.g., Fv, Fab, Fab′, F(ab′)₂, sdAb, scFv or other antigen-binding subsequences of antibodies) contain mostly human sequences but also (a) minimal sequence(s) derived from non-human immunoglobulin(s). For the most part, humanized antibodies are human immunoglobulins (recipient antibody) in which residues from one or more hypervariable regions (CDRs) of the recipient are replaced by residues from one or more hypervariable region of a non-human species (donor antibody) such as rodent (e.g., mouse, rat, or hamster), rabbit, or camelid (e.g., llama) having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody may also comprise at least a portion of an immunoglobulin constant region (Fc), typically that of a human immunoglobulin. For further details, see Jones et al. (1986) Nature, 321: 522-25; Reichmann et al. (1988) Nature, 332: 323-29; and Presta (1992) Curr. Op. Struct. Biol., 2: 593-96.

Humanized antibodies may be produced using transgenic animals such as mice that express human heavy and light chain genes, but are incapable of expressing the endogenous mouse immunoglobulin heavy and light chain genes. Winter describes an exemplary CDR grafting method that may be used to prepare the humanized antibodies described herein (see, U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol, 92: 3-16, 1982, and EP 239 400).

An antibody or fragment thereof may also be modified by specific deletion of human T cell epitopes in a process called “deimmunization.” Methods of deimmunization have been disclosed, for example, in WO1998052976A1 or WO2000034317A2. Briefly, the heavy and light chain variable domains of an antibody can be analyzed for peptides that bind to MHC class II; these peptides represent potential T cell epitopes. For detection of potential T cell epitopes, a computer modeling approach termed “peptide threading” can be applied, and in addition a database of human MHC class II binding peptides can be searched for motifs present in the VH and VL sequences. These motifs bind to any of the 18 major MHC class II DR allotypes, and thus constitute potential T cell epitopes. Potential T cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences are disclosed e.g., in Tomlinson, et al. (1992) J. Mol. Biol. 227:776-798; Cook, G. P. et al. (1995) Immunol. Today Vol. 16 (5): 237-242; and Tomlinson et al. (1995) EMBO J. 14: 4628-38. The V BASE directory provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

### E. Construct Formats

The first, second, and third antigen-binding sites may take various forms. In certain embodiments, the first, second, and/or third antigen-binding sites comprises two antibody variable domains (e.g., a VH and a VL). The VH and the VL can be mutated to introduce a disulfide bond (e.g., between H44 and L100) that stabilizes the antigen-binding site (see, Zhao et al. (2010) Int. J. Mol. Sci., 12(1):1-11). In certain embodiments, the first, second, and/or third antigen-binding sites comprises a single antibody variable domain (e.g., an sdAb).

In an antigen-binding site that contains a VH and a VL, the VH and the VL can be linked to form an scFv. The VH can be positioned N-terminal or C-terminal to the VL. The VH and the VL are typically linked through a linker, such as a peptide linker. Exemplary sequences of peptide linkers are provided in subsection I.F titled “Linkers.” In certain embodiments, the VH of an antigen-binding domain is connected to the VL of the antigen-binding domain through a peptide linker having an amino acid sequence listed in Table 4. In particular embodiments, the VH of an antigen-binding domain is connected to the VL of the antigen-binding domain through a peptide linker having the amino acid sequence of SEQ ID NO: 298, 299, or 302, wherein the VH is positioned N-terminal to the VL. In other particular embodiments, the VH of an antigen-binding domain is connected to the VL of the antigen-binding domain through a peptide linker having the amino acid sequence of SEQ ID NO: 298, 299, or 302, wherein the VH is positioned C-terminal to the VL.

Alternatively, the VH and the VL may be present on separate polypeptide chains, and the formation of a VH-VL complex may be facilitated by additional domains, such as antibody constant regions CH1 and CL. Accordingly, in certain embodiments, the multi-specific binding protein comprises an Fab comprising a VH and a VL disclosed herein.

In certain embodiments, a multi-specific binding protein of the present invention comprises a first antigen-binding site comprising a single antibody variable domain, a second antigen-binding site comprising a single antibody variable domain, and a third antigen-binding site comprising a single antibody variable domain. In certain embodiments, the multi-specific binding protein comprises a first antigen-binding site in an sdAb format, a second antigen-binding site in an sdAb format, and a third antigen-binding site in an sdAb format.

In certain embodiments, a multi-specific binding protein of the present invention comprises a first antigen-binding site comprising a single antibody variable domain, a second antigen-binding site comprising a single antibody variable domain, and a third antigen-binding site comprising two antibody variable domains. In certain embodiments, the multi-specific binding protein comprises a first antigen-binding site in an sdAb format, a second antigen-binding site in an sdAb format, and a third antigen-binding site in an scFv format.

In certain embodiments, a multi-specific binding protein of the present invention comprises a first antigen-binding site comprising a single antibody variable domain, a second antigen-binding site comprising two antibody variable domains, and a third antigen-binding site comprising a single antibody variable domain. In certain embodiments, the multi-specific binding protein comprises a first antigen-binding site in an sdAb format, a second antigen-binding site in an scFv format, and a third antigen-binding site in an sdAb format.

In certain embodiments, a multi-specific binding protein of the present invention comprises a first antigen-binding site comprising a single antibody variable domain, a second antigen-binding site comprising two antibody variable domains, and a third antigen-binding site comprising two antibody variable domains. In certain embodiments, the multi-specific binding protein comprises a first antigen-binding site in an sdAb format, a second antigen-binding site in an scFv format, and a third antigen-binding site in an scFv format.

In certain embodiments, a multi-specific binding protein of the present invention comprises a first antigen-binding site comprising two antibody variable domains, a second antigen-binding site comprising a single antibody variable domain, and a third antigen-binding site comprising a single antibody variable domain. In certain embodiments, the multi-specific binding protein comprises a first antigen-binding site in an scFv format, a second antigen-binding site in an sdAb format, and a third antigen-binding site in an sdAb format.

In certain embodiments, a multi-specific binding protein of the present invention comprises a first antigen-binding site comprising two antibody variable domains, a second antigen-binding site comprising a single antibody variable domain, and a third antigen-binding site comprising two antibody variable domains. In certain embodiments, the multi-specific binding protein comprises a first antigen-binding site in an scFv format, a second antigen-binding site in an sdAb format, and a third antigen-binding site in an scFv format.

In certain embodiments, a multi-specific binding protein of the present invention comprises a first antigen-binding site comprising two antibody variable domains, a second antigen-binding site comprising two antibody variable domains, and a third antigen-binding site comprising a single antibody variable domain. In certain embodiments, the multi-specific binding protein comprises a first antigen-binding site in an scFv format, a second antigen-binding site in an scFv format, and a third antigen-binding site in an sdAb format.

In certain embodiments, a multi-specific binding protein of the present invention comprises a first antigen-binding site comprising two antibody variable domains, a second antigen-binding site comprising two antibody variable domains, and a third antigen-binding site comprising two antibody variable domains. In certain embodiments, the multi-specific binding protein comprises a first antigen-binding site in an scFv format, a second antigen-binding site in an scFv format, and a third antigen-binding site in an scFv format.

The three antigen-binding sites of the multi-specific binding protein can be linked in any one of the following orientations in an amino-to-carboxyl direction:
(i) the first antigen-binding site (CD19 binding domain)—the second antigen-binding site (CD3 binding domain)—the third antigen-binding site (serum albumin binding domain);
(ii) the first antigen-binding site (CD19 binding domain)—the third antigen-binding site (serum albumin binding domain)—the second antigen-binding site (CD3 binding domain);
(iii) the second antigen-binding site (CD3 binding domain)—the first antigen-binding site (CD19 binding domain)—the third antigen-binding site (serum albumin binding domain);
(iv) the second antigen-binding site (CD3 binding domain)—the third antigen-binding site (serum albumin binding domain)—the first antigen-binding site (CD19 binding domain);
(v) the third antigen-binding site (serum albumin binding domain)—the first antigen-binding site (CD19 binding domain)—the second antigen-binding site (CD3 binding domain); and
(vi) the third antigen-binding site (serum albumin binding domain)—the second antigen-binding site (CD3 binding domain)—the first antigen-binding site (CD19 binding domain), wherein the dashes above represent a peptide bond and/or a linker (e.g., peptide linker).

In certain embodiments, the third antigen-binding site is not positioned between the first antigen-binding site and the second antigen-binding site. In certain embodiments, the third antigen-binding site is positioned N-terminal to both the first antigen-binding site and the second antigen-binding site or C-terminal to both the first antigen-binding site and the second antigen-binding site. In certain embodiments, the third antigen-binding site is positioned N-terminal to both the first antigen-binding site and the second antigen-binding site. In certain embodiments, the third antigen-binding site is positioned C-terminal to both the first antigen-binding site and the second antigen-binding site.

The position (N-terminal or C-terminal) of one antigen-binding site relative to another is determined under the definitions of “N-terminal” and “C-terminal” as known in the art if a single polypeptide chain comprises both antigen-binding sites. It is understood that if an antigen-binding site comprises two separate polypeptide chains, its position (N-terminal or C-terminal) relative to another antigen-binding site (either having a single polypeptide chain or two polypeptide chains) can be similarly determined if a single polypeptide chain comprises at least one polypeptide chain of the former and at least one polypeptide chain of the latter. It is further understood that if antigen-binding site A is N-terminal to antigen-binding site B and antigen-binding site B is N-terminal to antigen-binding site C, it is deemed that antigen-binding site A is positioned N-terminal to antigen-binding site C even if antigen-binding sites A and C are not present in any single, common polypeptide chain. More complex structures of multi-specific binding proteins are also contemplated, some of which may have orientations difficult to characterize using the terms of “N-terminal” and “C-terminal” as described above due to, for example, different relative positions of two antigen-binding sites on one polypeptide chain versus another polypeptide chain, or the presence of a loop structure.

According to the present invention, the multi-specific binding proteins and its constituent binding domains are in the form of one or more polypeptides. Such polypeptides may include proteinaceous parts and non-proteinaceous parts (e.g., chemical linkers or chemical cross-linking agents such as glutaraldehyde). In certain embodiments, a multi-specific binding protein of the present invention includes a first antigen-binding site, a second antigen-binding site, and a third antigen-binding site, all of which are linked together to form a single polypeptide chain. In certain embodiments, the first, second, and third antigen-binding sites take the forms of scFv and/or sdAb, for example, in a combination as described above, to form a single polypeptide chain.

### F. Linkers

As noted above, the antigen-binding sites of the multi-specific binding proteins of the present invention can be linked through a peptide bond or a linker (e.g., peptide linker). In certain embodiments, at least two adjacent antigen-binding sites are connected by a linker (e.g., peptide linker). In certain embodiments, each two adjacent antigen-binding sites are connected by a linker (e.g., peptide linker).

In certain embodiments, the three antigen-binding sites of the multi-specific binding protein can be linked by linkers (e.g., peptide linkers) denoted as L₁ and L₂ in any one of the following orientations in an amino-to-carboxyl direction:
(i) the first antigen-binding site (CD19 binding domain)—L₁—the second antigen-binding site (CD3 binding domain)—L₂—the third antigen-binding site (serum albumin binding domain);
(ii) the first antigen-binding site (CD19 binding domain)—L₁—the third antigen-binding site (serum albumin binding domain)—L₂—the second antigen-binding site (CD3 binding domain);
(iii) the second antigen-binding site (CD3 binding domain)—L₁—the first antigen-binding site (CD19 binding domain)—L₂—the third antigen-binding site (serum albumin binding domain);
(iv) the second antigen-binding site (CD3 binding domain)—L₁—the third antigen-binding site (serum albumin binding domain)—L₂—the first antigen-binding site (CD19 binding domain);
(v) the third antigen-binding site (serum albumin binding domain)—L₁—the first antigen-binding site (CD19 binding domain)—L₂—the second antigen-binding site (CD3 binding domain); and
(vi) the third antigen-binding site (serum albumin binding domain)—L₁—the second antigen-binding site (CD3 binding domain)—L₂—the first antigen-binding site (CD19 binding domain). It is appreciated that in a given construct, either L₁ or L₂ may be replaced with a peptide bond.

It is understood that if a single polypeptide chain comprises two adjacent antigen-binding sites, the peptide linker connecting the two antigen-binding sites represents the amino acid sequence between them. If an antigen-binding site comprises two separate polypeptide chains, one of which is present in a single, common polypeptide as an adjacent antigen-binding site or a polypeptide chain thereof, the peptide linker connecting the two antigen-binding sites represents the amino acid sequence between them in the common, single polypeptide.

In certain embodiments, the linkers L₁ and L₂ are peptide linkers. Suitable lengths of L₁ and L₂ can be independently selected. For example, in certain embodiments, L₁ and/or L₂ are about 50 or less amino acid residues in length. In certain embodiments, L₁ consists of about 50 or less amino acid residues. In certain embodiments, L₁ consists of about 20 or less amino acid residues. In certain embodiments, L₂ consists of about 50 or less amino acid residues. In certain embodiments, L₂ consists of about 20 or less amino acid residues. In certain embodiments, L₁ and L₂ independently consist of about 50 or less amino acid residues. In certain embodiments, L₁ and L₂ independently consist of about 20 or less amino acid residues.

In some embodiments, peptide linkers L₁ and L₂ have an optimized length and/or amino acid composition. In some embodiments, L₁ and L₂ are of the same length and have the same amino acid composition. In other embodiments, L₁ and L₂ are different. In certain embodiments, L₁ and/or L₂ are “short,” i.e., consist of 1, 2, 3, 4, 5, 6, 7, 8, 9, 10, 11 or 12 amino acid residues. Thus, in certain instances, the linkers consist of about 12 or less amino acid residues. In certain embodiments, L₁ and/or L₂ are “long,” e.g., consist of 15, 20 or 25 amino acid residues. In some embodiments, L₁ and/or L₂ consist of about 3 to about 15, for example 8, 9 or 10 contiguous amino acid residues.

Regarding the amino acid composition of L₁ and L₂, peptides are selected with properties that confer flexibility to multi-specific binding protein of the present invention, do not interfere with the binding domains as well as resist cleavage from proteases. For example, glycine and serine residues generally provide protease resistance. Examples of the linkers suitable for linking the domains in the multi-specific binding protein include but are not limited to (GS)ₙ, (GGS)ₙ, (GGGS)ₙ, (GGSG)ₙ, (GGSGG)ₙ, and (GGGGS)ₙ, wherein n is 1, 2, 3, 4, 5, 6, 7, 8, 9, 10, 11, 12, 13, 14, 15, 16, 17, 18, 19, or 20. In some embodiments, L₁ and/or L₂ are independently selected from the peptide sequences listed in table 4. In some embodiments, L₁ and/or L₂ are independently selected from SEQ ID NOs: 292, 293, 294, 295, 296, 297, 298, 299, 300, 301, or 302. In some embodiments, L₁ and/or L₂ are independently selected from SEQ ID NOs: 298, 299, and 302. In some embodiments, L₁ and/or L₂ comprise the amino acid sequence of SEQ ID NO: 298, 299, or 302. In some embodiments, L₁ and/or L₂ consist of the amino acid sequence of 298, 299, or 302. In some embodiments, L₁ and L₂ each comprise the amino acid sequence of SEQ ID NO: 298, 299, or 302. In some embodiments, L₁ and L₂ each consist of the amino acid sequence of SEQ ID NO: 298, 299, or 302.

TABLE 4
Sequences of Exemplary Peptide Linkers

| | SEQ ID | | |
|---|---|---|---|
| Linker | NO | Length | Amino Acid Sequence |
| (GS)₁₀ | 292 | 20 | GSGSGSGSGSGSGSGSGSGS |
| (GGS)₁₀ | 293 | 30 | GGSGGSGGSGGSGGSGGSGGSGGSGGSGGS |
| (GGGS)₁₀ | 294 | 40 | GGGSGGGSGGGSGGGSGGGSGGGSGGGSGGGSGGGS |
| | | | GGGS |
| (GGSG)₁₀ | 295 | 40 | GGSGGGSGGGSGGGSGGGSGGGSGGGSGGGSGGGSG |
| | | | GGSG |
| (GGSGG)₁₀ | 296 | 50 | GGSGGGGSGGGGSGGGGSGGGGSGGGGSGGGGSGG |
| | | | GGSGGGGSGGGGSGG |
| (GGGGS)₁₀ | 297 | 50 | GGGGSGGGGSGGGGSGGGGSGGGGSGGGGSGGGGS |
| | | | GGGGSGGGGSGGGGS |
| (GGGGS)₄ | 298 | 20 | GGGGSGGGGSGGGGSGGGGS |
| (GGGGS)₃ | 299 | 15 | GGGGSGGGGSGGGGS |
| (GGGGS)₂₀ | 300 | 100 | GGGGSGGGGSGGGGSGGGGSGGGGSGGGGSGGGGS |
| | | | GGGGSGGGGSGGGGSGGGGSGGGGSGGGGSGGGGS |
| | | | GGGGSGGGGSGGGGSGGGGSGGGGSGGGGS |
| (GGSGG)₂₀ | 301 | 100 | GGSGGGGSGGGGSGGGGSGGGGSGGGGSGGGGSGG |
| | | | GGSGGGGSGGGGSGGGGSGGGGSGGGGSGGGGSGG |
| | | | GGSGGGGSGGGGSGGGGSGGGGSGGGGSGG |
| Assymetrical | 302 | 9 | GGGGSGGGS |
| linker | | | |

A linker, such as a peptide linker disclosed herein, can also be used to connect the VH and VL of an scFv, as mentioned in subsection I.E titled “Construct Formats.”

### G. Binding Affinity

In certain embodiments, the multi-specific binding protein binds CD19 (e.g., human CD19), CD3 (e.g., human CD3 and/or Macaca CD3), and/or serum albumin (e.g., HSA) with a K_{D} of about 0.1 nM-about 100 μM. The K_{D} can be measured by a method known in the art, such as by SPR or by flow cytometry as described in Example 5 below.

In certain embodiments, the multi-specific binding protein binds CD19, CD3, and/or serum albumin with a K_{D} of lower than or equal to 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 90 pM, 80 pM, 70 pM, 60 pM, 50 pM, 40 pM, 30 pM, 20 pM, or 10 pM. For example, in certain embodiments, the multi-specific binding protein binds CD19, CD3, and/or serum albumin with a K_{D} of about 10 pM-about 1 nM, about 10 pM-about 0.9 nM, about 10 pM-about 0.8 nM, about 10 pM-about 0.7 nM, about 10 pM-about 0.6 nM, about 10 pM nM-about 0.5 nM, about 10 pM-about 0.4 nM, about 10 pM-about 0.3 nM, about 10 pM-about 0.2 nM, about 10 pM-about 0.1 nM, about 10 pM-about 50 pM, 0.1 nM-about 10 nM, about 0.1 nM-about 9 nM, about 0.1 nM-about 8 nM, about 0.1 nM-about 7 nM, about 0.1 nM-about 6 nM, about 0.1 nM-about 5 nM, about 0.1 nM-about 4 nM, about 0.1 nM-about 3 nM, about 0.1 nM-about 2 nM, about 0.1 nM-about 1 nM, about 0.1 nM-about 0.5 nM, about 0.5 nM-about 10 nM, about 1 nM-about 10 nM, about 2 nM-about 10 nM, about 3 nM-about 10 nM, about 4 nM-about 10 nM, about 5 nM-about 10 nM, about 6 nM-about 10 nM, about 7 nM-about 10 nM, about 8 nM-about 10 nM, or about 9 nM-about 10 nM.

In certain embodiments, the multi-specific binding protein binds CD19, CD3, and/or serum albumin with a K_{D} greater than or equal to 10 nM, 20 nM, 30 nM, 40 nM, 50 nM, 60 nM, 70 nM, 80 nM, 90 nM, or 100 nM. In certain embodiments, the multi-specific binding protein binds CD19, CD3, and/or serum albumin with a K_{D} of about 10 nM-about 1000 nM, about 10 nM-about 900 nM, about 10 nM-about 800 nM, about 10 nM-about 700 nM, about 10 nM-about 600 nM, about 10 nM-about 500 nM, about 10 nM-about 400 nM, about 10 nM-about 300 nM, about 10 nM-about 200 nM, about 10 nM-about 100 nM, about 10 nM-about 50 nM, about 50 nM-about 1000 nM, about 100 nM-about 1000 nM, about 200 nM-about 1000 nM, about 300 nM-about 1000 nM, about 400 nM-about 1000 nM, about 500 nM-about 1000 nM, about 600 nM-about 1000 nM, about 700 nM-about 1000 nM, about 800 nM-about 1000 nM, or about 900 nM-about 1000 nM.

In certain embodiments, the K_{D} of binding to CD19 or CD3 is measured in the absence of serum albumin (e.g., HSA). In certain embodiments, the K_{D} of binding to CD19 or CD3 is measured in substantial absence of serum albumin (e.g., HSA). In certain embodiments, the K_{D} of binding to CD19 or CD3 is measured in the presence of serum albumin (e.g., HSA), for example, in the presence of about 10 mg/mL, 15 mg/mL, 20 mg/mL, 25 mg/mL, 30 mg/mL, 35 mg/mL, 40 mg/mL, 45 mg/mL, or 50 mg/mL serum albumin (e.g., HSA).

In certain embodiments, the multi-specific binding protein of the present disclosure binds CD19, CD3, and/or serum albumin with a similar K_{D} value to that of the respective antigen-binding site alone or a monoclonal antibody having the same antigen-binding site. In certain embodiments, the K_{D} value of the multi-specific binding protein to CD19, CD3, and/or serum albumin is increased by no more than 1.5 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, or 50 fold compared to that of the respective antigen-binding site alone or a monoclonal antibody having the same antigen-binding site.

In certain embodiments, the multi-specific binding protein of the present disclosure binds CD19 and/or CD3 with a similar K_{D} value in the presence of serum albumin to that in the absence or substantial absence of serum albumin. In certain embodiments, the K_{D} value of the multi-specific binding protein for binding CD19 and/or CD3 in the presence of serum albumin is increased by no more than 1.5 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, or 50 fold compared to that in the absence or substantial absence of serum albumin.

### H. Therapeutic Activities of the Constructs

The multi-specific binding protein disclosed herein is designed to simultaneously bind B cells and T cells. Recruitment of T cells facilitates lysis of the B cells involving cytolytic synapse formation and delivery of perforin and granzymes. The engaged T cells are capable of serial target cell lysis, and are not affected by immune escape mechanisms interfering with peptide antigen processing and presentation, or clonal T cell differentiation; see, for example, WO2007042261A2. Accordingly, binding of the multi-specific binding proteins to the target B cells destroys the target cells and/or impairs the progression of B cell related diseases.

Cytotoxicity mediated by multi-specific binding proteins of the invention can be measured in various ways in vitro. Effector cells can be e.g., stimulated enriched (human) CD8 positive T cells or unstimulated (human) peripheral blood mononuclear cells (PBMC). If the target cells are of macaque origin or express or are transfected with macaque target cell surface antigen which is bound by the first domain, the effector cells should also be of macaque origin such as a macaque T cell line, e.g., 4119LnPx. The target cells should express CD19, e.g., human or macaque CD19. The target cells can be a cell line (such as CHO) which is stably or transiently transfected with CD19. Alternatively, the target cells can be a cell line naturally expressing CD19, such as B lymphocytes. The effector to target cell (E:T) ratio is usually about 10:1, but can also vary. Killing of the target cells can be measured in a ⁵¹Cr-release assay (incubation time of about 18 hours) or in a in a FACS-based cytotoxicity assay (incubation time of about 48 hours). Other methods of measuring cell death are well-known to the skilled person, such as MTT or MTS assays, ATP-based assays including bioluminescent assays, the sulforhodamine B (SRB) assay, WST assay, clonogenic assay and the ECIS technology.

In certain embodiments, the cytotoxic activity mediated by the multi-specific binding protein disclosed herein is measured in a cell-based cytotoxicity assay described above. It is represented by the EC₅₀ value, which corresponds to the half maximal effective concentration (concentration of the multi-specific binding protein which induces a cytotoxic response halfway between the baseline and maximum). In certain embodiments, the EC₅₀ value of the multi-specific binding proteins is ≤5000 pM, for example, ≤4000 pM, ≤3000 pM, ≤2000 pM, ≤1000 pM, ≤500 pM, ≤400 pM, ≤300 pM, ≤200 pM, ≤100 pM, ≤50 pM, ≤20 pM, ≤10 pM, ≤5 pM, ≤4 pM, ≤3 pM, ≤2 pM, or ≤1 pM.

It is understood that an EC₅₀ value is generally lower when stimulated/enriched CD8⁺ T cells are used as effector cells, compared with unstimulated PBMC. It is further understood that the EC₅₀ value is generally lower when the target cells express a high level of the target cell surface antigen compared with a low level of the target antigen. For example, when stimulated/enriched human CD8⁺ T cells are used as effector cells (and either target cell surface antigen transfected cells such as CHO cells or target cell surface antigen positive human cell lines are used as target cells), the EC₅₀ value of multi-specific binding protein is ≤1000 pM, for example, ≤500 pM, ≤250 pM, ≤100 pM, ≤50 pM, ≤10 pM, or ≤5 pM. When human PBMCs are used as effector cells, the EC₅₀ value of the multi-specific binding protein is ≤5000 pM, for example, ≤4000 pM, ≤2000 pM, ≤1000 pM, ≤500 pM, ≤200 pM, ≤150 pM, ≤100 pM, ≤50 pM, ≤10 pM, or ≤5 pM. When a macaque T cell line such as LnPx4119 is used as effector cells, and a macaque target cell surface antigen transfected cell line such as CHO cells is used as target cell line, the EC₅₀ value of the multi-specific binding protein is ≤2000 pM, for example, ≤1500 pM, ≤100 pM, ≤500 pM, ≤300 pM, ≤250 pM, ≤100 pM, ≤50 pM, ≤10 pM, or ≤5 pM.

Accordingly, in certain embodiments, the EC₅₀ value is measured using stimulated/enriched human CD8⁺ T cells as effector cells. In certain embodiments, the EC₅₀ value is measured using human PBMCs as effector cells. In certain embodiments, the EC₅₀ value is measured using a macaque T cell line such as LnPx4119 as effector cells and cells (e.g., CHO cells) engineered to express macaque CD19 as target cells.

In certain embodiments, the multi-specific binding protein of the present invention does not induce or mediate lysis of cells that do not express CD19. The term “does not induce lysis” or “does not mediate lysis,” or grammatical equivalents thereof, means that the multi-specific binding protein, at a concentration of up to 500 nM, does not induce or mediate lysis of more than 30%, for example, no more than 30%, 25%, 20%, 15%, 10%, 9%, 8%, 7%, 6% or 5% of cells that do not express CD19, whereby lysis of a cell line that expresses CD19 is set to be 100%.

In certain embodiments, a multi-specific binding protein disclosed herein is more effective in in killing CD19-expressing cells (e.g., cancer cells) than the corresponding respective anti-CD19 or anti-CD3 monoclonal antibody at the same molar concentration. In certain embodiments, the multi-specific binding protein is more effective in killing CD19-expressing cells (e.g., cancer cells) than a combination of the corresponding respective anti-CD19 and anti-CD3 monoclonal antibodies each at the same molar concentration.

The cytotoxic activity of the multi-specific binding protein can be measured in the presence or absence of serum albumin (e.g., HSA). In certain embodiments, the cytotoxic activity disclosed above is measured in the absence of serum albumin (e.g., HSA). In certain embodiments, the cytotoxic activity disclosed above is measured in substantial absence of serum albumin (e.g., HSA). In certain embodiments, the cytotoxic activity disclosed above is measured in the presence of serum albumin (e.g., HSA), for example, in the presence of about 10 mg/mL, 15 mg/mL, 20 mg/mL, 25 mg/mL, 30 mg/mL, 35 mg/mL, 40 mg/mL, 45 mg/mL, or 50 mg/mL serum albumin (e.g., HSA).

In certain embodiments, the multi-specific binding protein of the present disclosure kills CD19-expressing cells with a similar EC₅₀ value in the presence of serum albumin to that in the absence or substantial absence of serum albumin. In certain embodiments, the EC₅₀ value of the multi-specific binding protein for killing CD19-expressing cells in the presence of serum albumin is increased by no more than 1.5 fold, 2 fold, 3 fold, 4 fold, 5 fold, 6 fold, 7 fold, 8 fold, 9 fold, 10 fold, 15 fold, 20 fold, 25 fold, 30 fold, 35 fold, 40 fold, 45 fold, or 50 fold compared to that in the absence or substantial absence of serum albumin. It is understood that the presence of serum albumin (e.g., about 10 mg/mL, 15 mg/mL, 20 mg/mL, 25 mg/mL, 30 mg/mL, 35 mg/mL, 40 mg/mL, 45 mg/mL, or 50 mg/mL serum albumin) may also alter the EC₅₀ value of a multi-specific binding protein nonspecifically. The nonspecific effect can be assessed by comparing the EC₅₀ values of a control protein, which does not contain a serum albumin binding domain, in the presence and absence of serum albumin. In certain embodiments, the fold change is offset by the nonspecific effect of serum albumin on a control protein, such as a bispecific protein that binds CD19 and CD3.

### I. Construct Size

In certain embodiments, the molecular weight of the multi-specific binding protein is from about 40 kD to about 100 kD. In certain embodiments, the molecular weight of the multi-specific binding protein is at least 60 kD, at least 65 kD, at least 70 kD, at least 75 kD, at least 80 kD, at least 85 kD, at least 90 kD, or at least 95 kD. It is understood that smaller size generally contributes to faster diffusion and tissue penetration, but size reduction may not be as critical for the purpose of treating the indications with substantial presence of target cells (e.g., cancer cells) in the blood.

In certain embodiments, the molecular weight of the multi-specific binding protein is from about about 40 kD to about 90 kD, from about 40 kD to about 80 kD, from about 40 kD to about 70 kD, from about 40 kD to about 60 kD, from about 40 kD to about 50 kD, from about 50 kD to about 100 kD, from about 50 kD to about 90 kD, from about 50 kD to about 80 kD, from about 50 kD to about 70 kD, from about 50 kD to about 60 kD, from about 60 kD to about 100 kD, from about 60 kD to about 90 kD, from about 60 kD to about 80 kD, from about 60 kD to about 70 kD, from about 65 kD to about 100 kD, from about 65 kD to about 90 kD, from about 65 kD to about 80 kD, from about 65 kD to about 70 kD, from about 70 kD to about 100 kD, from about 70 kD to about 90 kD, from about 70 kD to about 80 kD, from about 80 kD to about 100 kD, from about 80 kD to about 90 kD, or from about 90 kD to about 100 kD. In certain embodiments, the multi-specific binding protein is lower than 40 kD. In certain embodiments, the multi-specific binding protein is about 50 kD-about 90 kD, about 50 kD-about 80 kD, about 50 kD-about 70 kD, about 50 kD-about 60 kD, about 60 kD-about 90 kD, about 60 kD-about 80 kD, about 60 kD-about 70 kD, about 65 kD-about 90 kD, about 65 kD-about 80 kD, about 65 kD-about 70 kD, about 70 kD-about 90 kD, or about 70 kD-about 80 kD.

### J. Serum Half-life

Fusion proteins have been developed to increase the in vivo half-life of a small protein, particularly an antibody fragment. For example, fusion with a heterodimeric antibody Fc region, such as an Fc with one or more mutations that extend the in vivo half-life, is described in U.S. Patent Application Publication Nos. US20140302037A1, US20140308285A1, and PCT Publication Nos. WO2014144722A2, WO2014151910A1 and WO2015048272A1. An alternative strategy is fusion with human serum albumin (HSA) or an HSA-binding peptide (see, e.g., PCT Publication Nos. WO2013128027A1 and WO2014140358A1). The neonatal Fc receptor (FcRn) appears to be involved in prolonging the life-span of albumin in circulation (see, Chaudhury et al. (2003) J. Exp. Med., 3: 315-22). Albumin and IgG bind noncooperatively to distinct sites of FcRn and form a tri-molecular (see id.). Binding of human FcRn to HSA and to human IgG is pH dependent, stronger at acidic pH and weaker at neutral or physiological pH (see id.). This observation suggests that proteins and protein complexes containing albumin, similar to those containing IgG (particularly Fc), are protected from degradation through pH-sensitive interaction with FcRn (see id.). Using surface plasmon resonance (SPR) to measure the capacity of individual HSA domains to bind immobilized soluble human FcRn, it has been shown that FcRn and albumin interact via the D-III domain of albumin in a pH-dependent manner, on a site distinct from the IgG binding site (see, Chaudhury et al. (2006) Biochemistry 45:4983-90 and PCT Publication No. WO2008068280A1).

The present disclosure provides multi-specific binding proteins with extended half-life. In certain embodiments, the multi-specific binding protein has a serum half-life of at least 24, 36, 48, 60, 72, 84, or 96 hours. In certain embodiments, the multi-specific binding protein has a serum half-life of at least about 50 hours. In certain embodiments, the multi-specific binding protein has a serum half-life of at least about 100 hours. Methods of measuring serum half-life are known in the art, and exemplary methods are described in Example 4. In certain embodiments, the serum half-life is measured in a non-human primate. In certain embodiments, the serum half-life is measured in human.

In certain embodiments, 50 hours after intravenous administration to a subject, the serum concentration of the multi-specific binding protein is at least 10%, at least 20%, at least 30%, at least 40%, at least 50%, at least 60%, at least 70%, at least 80%, or at least 90% of the serum concentration of the multi-specific binding protein 1 hour after the administration in said subject.

In certain embodiments, the multi-specific binding protein has a serum half-life that is at least 20% longer than a control multi-specific binding protein, wherein the control multi-specific binding protein includes a first domain identical to the first antigen-binding site of the multi-specific binding protein, a second domain identical to the second antigen-binding site of the multi-specific binding protein, but not a third domain identical or substantially identical to the third antigen-binding site of the multi-specific binding protein. In certain embodiments, the control multi-specific binding protein is identical to the multi-specific binding protein but for the absence of the third antigen-binding site. In certain embodiments, the serum half-life of the multi-specific binding protein is at least 30%, at least 40%, at least 50%, at least 60%, at least 70%, at least 80%, or at least 90% longer than the serum half-life of the control multi-specific binding protein. In certain embodiments, the serum half-life of the multi-specific binding protein is longer than the serum half-life of the control multi-specific binding protein by at least 2 fold, at least 3 fold, at least 4 fold, at least 5 fold, at least 6 fold, at least 7 fold, at least 8 fold, at least 9 fold, or at least 10 fold.

### II. Methods of Preparation

The multi-specific binding proteins described above can be made using recombinant DNA technology well known to a skilled person in the art. For example, one or more isolated polynucleotides encoding the multi-specific binding protein can be ligated to other appropriate nucleotide sequences, including, for example, constant region coding sequences, and expression control sequences, to produce conventional gene expression constructs (i.e., expression vectors) encoding the desired multi-specific binding proteins. Production of defined gene constructs is within routine skill in the art.

Nucleic acids encoding desired multi-specific binding proteins can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the multi-specific binding proteins.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. In embodiments involving fusion proteins comprising an antibody or portion thereof, the expression vector optionally contains sequences encoding all or part of a constant region, enabling an entire, or a part of, a heavy or light chain to be expressed. The gene construct can be introduced into eukaryotic host cells using conventional techniques.

The multi-specific binding protein disclosed herein may comprise a single polypeptide chain. In this instance, a host cell can be transfected with a single vector expressing the polypeptide (e.g., containing an expression control sequence operably linked to a nucleotide sequence encoding the polypeptide). Alternatively, multi-specific binding protein disclosed herein may comprise two or more polypeptides. In this instance, a host cell can be co-transfected with more than one expression vector, for example, one expression vector expressing each polypeptide. A host cell can also be transfected with a single expression vector that expresses the two or more polypeptides. For example, the coding sequences of the two or more polypeptides can be operably linked to different expression control sequences (e.g., promoter, enhancer, and/or internal ribosome entry site (IRES)). The coding sequences of the two or more polypeptides can also be separated by a ribosomal skipping sequence or self-cleaving sequence, such as a 2A peptide.

In certain embodiments, in order to express a multi-specific binding protein, an N-terminal signal sequence is included in the protein construct. Exemplary N-terminal signal sequences include signal sequences from interleukin-2, CD-5, IgG kappa light chain, trypsinogen, serum albumin, and prolactin.

After transfection, single clones can be isolated for cell bank generation using methods known in the art, such as limited dilution, ELISA, FACS, microscopy, or Clonepix. Clones can be cultured under conditions suitable for bio-reactor scale-up and maintained expression of the multi-specific binding proteins.

The multi-specific binding proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### III. Pharmaceutical Compositions

The present disclosure also features pharmaceutical compositions that contain a therapeutically effective amount of the multi-specific binding proteins described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29).

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(—)-3-hydroxybutyric acid. Sustained release compositions may also include liposomes that can be prepared by any of several methods known in the art.

Pharmaceutical compositions containing a multi-specific binding protein disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, a recombinant human sialidase, a recombinant human sialidase fusion protein, or an antibody conjugate disclosed herein is administered by IV infusion. In certain embodiments, a recombinant human sialidase, a recombinant human sialidase fusion protein, or an antibody conjugate disclosed herein is administered by intratumoral injection. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor® EL™ (BASF®, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

An intravenous drug delivery formulation may be contained in a syringe, pen, or bag. In certain embodiments, the bag may be connected to a channel comprising a tube and/or a needle. In certain embodiments, the formulation may be a lyophilized formulation or a liquid formulation. In certain embodiments, the formulation may freeze-dried (lyophilized) and contained in about 12-60 vials. In certain embodiments, the formulation may be freeze-dried and 45 mg of the freeze-dried formulation may be contained in one vial. In certain embodiments, the about 40 mg-about 100 mg of freeze-dried formulation may be contained in one vial. In certain embodiments, freeze dried formulation from 12, 27, or 45 vials are combined to obtained a therapeutic dose of the protein in the intravenous drug formulation. In certain embodiments, the formulation may be a liquid formulation and stored as about 250 mg/vial to about 1,000 mg/vial. In certain embodiments, the formulation may be a liquid formulation and stored as about 600 mg/vial. In certain embodiments, the formulation may be a liquid formulation and stored as about 250 mg/vial.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including the protein of the present disclosure in a pH-buffered solution. The buffer of this invention may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g. sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/ml of citric acid (e.g., 1.305 mg/ml), about 0.3 mg/ml of sodium citrate (e.g., 0.305 mg/ml), about 1.5 mg/ml of disodium phosphate dihydrate (e.g., 1.53 mg/ml), about 0.9 mg/ml of sodium dihydrogen phosphate dihydrate (e.g., 0.86), and about 6.2 mg/ml of sodium chloride (e.g., 6.165 mg/ml). In certain embodiments, the buffer system includes 1-1.5 mg/ml of citric acid, 0.25 to 0.5 mg/ml of sodium citrate, 1.25 to 1.75 mg/ml of disodium phosphate dihydrate, 0.7 to 1.1 mg/ml of sodium dihydrogen phosphate dihydrate, and 6.0 to 6.4 mg/ml of sodium chloride. In certain embodiments, the pH of the formulation is adjusted with sodium hydroxide.

A polyol, which acts as a tonicifier and may stabilize the multi-specific binding protein, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/ml. In certain embodiments, the concentration of mannitol may be about 7.5 to 15 mg/ml. In certain embodiments, the concentration of mannitol may be about 10-14 mg/ml. In certain embodiments, the concentration of mannitol may be about 12 mg/ml. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th edi., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In embodiments, the protein product of the present disclosure is formulated as a liquid formulation. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments, the liquid formulation may be diluted with 0.9% saline solution.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

The multi-specific binding protein may be lyophilized to produce a lyophilized formulation including the proteins and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5. In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide. Before lyophilization, the pH of the solution containing the protein of the present disclosure may be adjusted between 6 to 8. In certain embodiments, the pH range for the lyophilized drug product may be from 7 to 8.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5,000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

In general, dosages based on body weight are from about 0.01 μg to about 100 mg per kg of body weight, such as about 0.01 μg to about 100 mg/kg of body weight, about 0.01 g to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 g to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, about 50 mg to about 100 mg/kg of body weight.

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

### IV. Therapeutic Applications

It is contemplated that the multi-specific binding proteins can be used either alone or in combination with other therapeutic agents.

### A. Indications

The present disclosure provides methods for the treatment or amelioration of a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease in a subject in need thereof, the method comprising administration of the multi-specific binding proteins described herein.

In certain embodiments, the cancer to be treated is non-Hodgkin's lymphoma, such as a B-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, chronic lymphocytic leukemia, or primary central nervous system lymphoma. In certain other embodiments, the cancer to be treated is multiple myeloma. In certain other embodiments, the cancer to be treated is acute lymphoblastic leukemia (ALL). In certain embodiments, the ALL is relapsed/refractory adult and pediatric ALL.

### B. Combination Therapies

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In one aspect, the present disclosure provides a method of treating a subject by the administration of a second therapeutic agent in combination with one or more of the multi-specific binding proteins described herein.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

An additional class of agents that may be used as part of a combination therapy in treating cancer is immune checkpoint inhibitors. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

In certain embodiments, the checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (available under the tradename Opdivo® from Bristol-Myers Squibb Co.), pembrolizumab (available under the tradename Keytruda® from Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (available under the tradename Tecentriq® from Genentech®), duvalumab (AstraZeneca®), MEDI4736, avelumab, and BMS 936559 (Bristol Myers Squibb Co.).

In certain embodiments, a method or composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

In certain embodiments, a method or composition described herein is administered in combination with (i) a PD-1 or PD-L1 inhibitor, e.g., a PD-1 or PD-L1 inhibitor disclosed herein, and (ii) CTLA-4 inhibitor, e.g., a CTLA-4 inhibitor disclosed herein.

In certain embodiments, a method or composition described herein is administered in combination with an IDO inhibitor. Exemplary IDO inhibitors include 1-methyl-D-tryptophan (known as indoximod), epacadostat (INCB24360), navoximod (GDC-0919), and BMS-986205.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

It is understood that the multi-specific antibody disclosed herein, which is designed to activate T lymphocytes, may cause side effects such as neurotoxicity. Accordingly, in certain embodiments, the second therapeutic agent that can be used in combination with the multi-specific binding protein comprises an agent that mitigates a side effect of the multi-specific binding protein, e.g., reduces neurotoxicity. In certain embodiments, the second therapeutic agent inhibits T cell trafficking, for example, reduces or inhibits immune cells from crossing the blood-brain barrier. Non-limiting examples of such therapeutic agents include antagonists (e.g., antagonistic antibodies) of adhesion molecules on immune cells (e.g., a4 integrin), such as natalizumab. In certain embodiments, the second therapeutic agent increases the internalization of a sphingosine-1-phosphate (SIP) receptor (e.g., S1PR1 or S1PR5), such as fingolimod or ozanimod. In certain embodiments, the second therapeutic agent is a nitric oxide synthase (NOS) inhibitor, such as ronopterin, cindunistat, A-84643, ONO-1714, L-NOARG, NCX-456, VAS-2381, GW-273629, NXN-462, CKD-712, KD-7040, or guanidinoethyldisulfide. In certain embodiments, the second therapeutic agent is an antagonist of CSF1 or CSF1R, such as pexidartinib, emactuzumab, cabiralizumab, LY-3022855, JNJ-40346527, or MCS110. Additional non-limiting examples of the second therapeutic agents include pentosan polysulfate, minocycline, anti-ICAM-1 antibodies, anti-P-selectin antibodies, anti-CD11a antibodies, anti-CD162 antibodies, and anti-IL-6R antibodies (e.g., tocilizumab).

The amount of the multi-specific binding protein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multi-specific binding protein may be administered during a time when the additional therapeutic agent(s) exerts its prophylactic or therapeutic effect, or vice versa.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

The description above describes multiple aspects and embodiments of the invention. The patent application specifically contemplates all combinations and permutations of the aspects and embodiments.

### Examples

The invention now being generally described, will be more readily understood by reference to the following examples, which are included merely for purposes of illustration of certain aspects and embodiments of the present invention, and is not intended to limit the invention.

### Example 1. Production of Multi-Specific Binding Proteins

This example describes the production and purification of multi-specific binding proteins.

Nucleic acids encoding single-chain multi-specific binding proteins (see Table 5) were constructed and codon optimized for expression in human cells and cloned into a mammalian expression vector following standard procedures. Following sequence verification, the expression vectors, in the form of plasmids, were prepared in sufficient quantity for transfection using Plasmid Plus purification kits (Qiagen®). Human embryonic kidney 293 (HEK 293) cells were passaged to appropriate density for transient transfection. Cells were transiently transfected with the expression vectors and cultured for six days.

The amino acid sequences of the various multi-specific binding proteins are summarized in Table 5. Constructs tAbK27 to tAbSK32 each contained an anti-CD19 scFv having the amino acid sequence set forth in SEQ ID NO: 9, an anti-CD3 scFv having the amino acid sequence set forth in SEQ ID NO: 105, and an anti-HSA sdAb having the amino acid sequence set forth in SEQ ID NO: 121. Constructs tAb0033 to tAb0038 each contained an anti-CD19 scFv having the amino acid sequence set forth in SEQ ID NO: 18, an anti-CD3 scFv having the amino acid sequence set forth in SEQ ID NO: 105, and an anti-HSA sdAb having the amino acid sequence set forth in SEQ ID NO: 121.

TABLE 5
Exemplary Multi-specific Binding Proteins

| Construct | Format | Amino Acid Sequence |
|---|---|---|
| tAb0027 | CD19:CD3: | QVQLVQSGAEVKKPGASVKVSCKASGYTFTDYIMHWVRQAPGQ |
| | HSA | GLEWMGYINPYNDGSKYTEKFQGRVTMTSDTSISTAYMELSRLRS |
| | | DDTAVYYCARGTYYYGPQLFDYWGQGTTVTVSSGGGGSGGGGS |
| | | GGGGSDIVMTQTPLSLSVTPGQPASISCKSSQSLETSTGTTYLNWY |
| | | LQKPGQSPQLLIYRVSKRFSGVPDRFSGSGSGTDFTLKISRVEAEDV |
| | | GVYYCLQLLEDPYTFGQGTKLEIKGGGGSGGGSDIVMTQSPDSLA |
| | | VSLGERATINCKSSQSLLNARTGKNYLAWYQQKPGQPPKLLIYWA |
| | | STRESGVPDRFSGSGSGTDFTLTISSLQAEDVAVYYCKQSYSRRTF |
| | | GGGTKVEIKGGGGSGGGGSGGGGSGGGGSQVQLVQSGAEVKKP |
| | | GASVKVSCKASGFNIKDYYMHWVRQAPGQRLEWMGWIDLENAN |
| | | TIYDAKFQGRVTITRDTSASTAYMELSSLRSEDTAVYYCARDAYG |
| | | RYFYDVWGQGTLVTVSSGGGGSGGGSEVQLVESGGGLVQPGNSL |
| | | RLSCAASGFTFSSFGMSWVRQAPGKGLEWVSSISGSGSDTLYADS |
| | | VKGRFTISRDNAKTTLYLQMNSLRPEDTAVYYCTIGGSLSRSSQGT |
| | | LVTVSSHHHHHHHHHH (SEQ ID NO: 303) |
| tAb0029 | CD3:CD19: | DIVMTQSPDSLAVSLGERATINCKSSQSLLNARTGKNYLAWYQQK |
| | HSA | PGQPPKLLIYWASTRESGVPDRESGSGSGTDFTLTISSLQAEDVAV |
| | | YYCKQSYSRRTFGGGTKVEIKGGGGSGGGGSGGGGSGGGGSQVQ |
| | | LVQSGAEVKKPGASVKVSCKASGFNIKDYYMHWVRQAPGQRLE |
| | | WMGWIDLENANTIYDAKFQGRVTITRDTSASTAYMELSSLRSEDT |
| | | AVYYCARDAYGRYFYDVWGQGTLVTVSSGGGGSGGGSQVQLVQ |
| | | SGAEVKKPGASVKVSCKASGYTFTDYIMHWVRQAPGQGLEWMG |
| | | YINPYNDGSKYTEKFQGRVTMTSDTSISTAYMELSRLRSDDTAVY |
| | | YCARGTYYYGPQLFDYWGQGTTVTVSSGGGGSGGGGSGGGGSDI |
| | | VMTQTPLSLSVTPGQPASISCKSSQSLETSTGTTYLNWYLQKPGQS |
| | | PQLLIYRVSKRFSGVPDRFSGSGSGTDFTLKISRVEAEDVGVYYCL |
| | | QLLEDPYTFGQGTKLEIKGGGGSGGGSEVQLVESGGGLVQPGNSL |
| | | RLSCAASGFTFSSFGMSWVRQAPGKGLEWVSSISGSGSDTLYADS |
| | | VKGRFTISRDNAKTTLYLQMNSLRPEDTAVYYCTIGGSLSRSSQGT |
| | | LVTVSSHHHHHHHHHH (SEQ ID NO: 304) |
| tAb0030 | CD3:HSA: | DIVMTQSPDSLAVSLGERATINCKSSQSLLNARTGKNYLAWYQQK |
| | CD19 | PGQPPKLLIYWASTRESGVPDRESGSGSGTDFTLTISSLQAEDVAV |
| | | YYCKQSYSRRTFGGGTKVEIKGGGGSGGGGSGGGGSGGGGSQVQ |
| | | LVQSGAEVKKPGASVKVSCKASGFNIKDYYMHWVRQAPGQRLE |
| | | WMGWIDLENANTIYDAKFQGRVTITRDTSASTAYMELSSLRSEDT |
| | | AVYYCARDAYGRYFYDVWGQGTLVTVSSGGGGSGGGSEVQLVE |
| | | SGGGLVQPGNSLRLSCAASGFTFSSFGMSWVRQAPGKGLEWVSSI |
| | | SGSGSDTLYADSVKGRFTISRDNAKTTLYLQMNSLRPEDTAVYYC |
| | | TIGGSLSRSSQGTLVTVSSGGGGSGGGSQVQLVQSGAEVKKPGAS |
| | | VKVSCKASGYTFTDYIMHWVRQAPGQGLEWMGYINPYNDGSKY |
| | | TEKFQGRVTMTSDTSISTAYMELSRLRSDDTAVYYCARGTYYYGP |
| | | QLFDYWGQGTTVTVSSGGGGSGGGGSGGGGSDIVMTQTPLSLSV |
| | | TPGQPASISCKSSQSLETSTGTTYLNWYLQKPGQSPQLLIYRVSKRF |
| | | SGVPDRFSGSGSGTDFTLKISRVEAEDVGVYYCLQLLEDPYTFGQG |
| | | TKLEIKHHHHHHHHHH (SEQ ID NO: 305) |
| tAb0031 | HSA:CD3: | EVQLVESGGGLVQPGNSLRLSCAASGFTFSSFGMSWVRQAPGKGL |
| | CD19 | EWVSSISGSGSDTLYADSVKGRFTISRDNAKTTLYLQMNSLRPEDT |
| | | AVYYCTIGGSLSRSSQGTLVTVSSGGGGSGGGSDIVMTQSPDSLAV |
| | | SLGERATINCKSSQSLLNARTGKNYLAWYQQKPGQPPKLLIYWAS |
| | | TRESGVPDRFSGSGSGTDFTLTISSLQAEDVAVYYCKQSYSRRTFG |
| | | GGTKVEIKGGGGSGGGGSGGGGSGGGGSQVQLVQSGAEVKKPG |
| | | ASVKVSCKASGFNIKDYYMHWVRQAPGQRLEWMGWIDLENANT |
| | | IYDAKFQGRVTITRDTSASTAYMELSSLRSEDTAVYYCARDAYGR |
| | | YFYDVWGQGTLVTVSSGGGGSGGGSQVQLVQSGAEVKKPGASV |
| | | KVSCKASGYTFTDYIMHWVRQAPGQGLEWMGYINPYNDGSKYT |
| | | EKFQGRVTMTSDTSISTAYMELSRLRSDDTAVYYCARGTYYYGPQ |
| | | LFDYWGQGTTVTVSSGGGGSGGGGSGGGGSDIVMTQTPLSLSVTP |
| | | GQPASISCKSSQSLETSTGTTYLNWYLQKPGQSPQLLIYRVSKRFS |
| | | GVPDRFSGSGSGTDFTLKISRVEAEDVGVYYCLQLLEDPYTFGQG |
| | | TKLEIKHHHHHHHHHH (SEQ ID NO: 306) |
| tAb0032 | HSA:CD19: | EVQLVESGGGLVQPGNSLRLSCAASGFTFSSFGMSWVRQAPGKGL |
| | CD3 | EWVSSISGSGSDTLYADSVKGRFTISRDNAKTTLYLQMNSLRPEDT |
| | | AVYYCTIGGSLSRSSQGTLVTVSSGGGGSGGGSQVQLVQSGAEVK |
| | | KPGASVKVSCKASGYTFTDYIMHWVRQAPGQGLEWMGYINPYN |
| | | DGSKYTEKFQGRVTMTSDTSISTAYMELSRLRSDDTAVYYCARGT |
| | | YYYGPQLFDYWGQGTTVTVSSGGGGSGGGGSGGGGSDIVMTQTP |
| | | LSLSVTPGQPASISCKSSQSLETSTGTTYLNWYLQKPGQSPQLLIYR |
| | | VSKRFSGVPDRFSGSGSGTDFTLKISRVEAEDVGVYYCLQLLEDPY |
| | | TFGQGTKLEIKGGGGSGGGSDIVMTQSPDSLAVSLGERATINCKSS |
| | | QSLLNARTGKNYLAWYQQKPGQPPKLLIYWASTRESGVPDRFSGS |
| | | GSGTDFTLTISSLQAEDVAVYYCKQSYSRRTFGGGTKVEIKGGGG |
| | | SGGGGSGGGGSGGGGSQVQLVQSGAEVKKPGASVKVSCKASGEN |
| | | IKDYYMHWVRQAPGQRLEWMGWIDLENANTIYDAKFQGRVTITR |
| | | DTSASTAYMELSSLRSEDTAVYYCARDAYGRYFYDVWGQGTLVT |
| | | VSSHHHHHHHHHH (SEQ ID NO: 307) |
| tAb0033 | CD19:CD3: | QVQLQESGPGLVKPSQTLSLTCTVSGGSISTSGMGVGWIRQHPGK |
| | HSA | GLEWIGHIWWDDDKRYNPALKSRVTISVDTSKNQFSLKLSSVTAA |
| | | DTAVYYCARMELWSYYFDYWGQGTLVTVSSGGGGSGGGGSGGG |
| | | GSEIVLTQSPATLSLSPGERATLSCSASSSVSYMHWYQQKPGQAPR |
| | | LLIYDTSKLASGIPARFSGSGSGTDFTLTISSLEPEDFAVYYCFQGSV |
| | | YPFTFGQGTKLEIKRGGGGSGGGSDIVMTQSPDSLAVSLGERATIN |
| | | CKSSQSLLNARTGKNYLAWYQQKPGQPPKLLIYWASTRESGVPD |
| | | RFSGSGSGTDFTLTISSLQAEDVAVYYCKQSYSRRTFGGGTKVEIK |
| | | GGGGSGGGGSGGGGSGGGGSQVQLVQSGAEVKKPGASVKVSCK |
| | | ASGFNIKDYYMHWVRQAPGQRLEWMGWIDLENANTIYDAKFQG |
| | | RVTITRDTSASTAYMELSSLRSEDTAVYYCARDAYGRYFYDVWG |
| | | QGTLVTVSSGGGGSGGGSEVQLVESGGGLVQPGNSLRLSCAASGF |
| | | TFSSFGMSWVRQAPGKGLEWVSSISGSGSDTLYADSVKGRFTISRD |
| | | NAKTTLYLQMNSLRPEDTAVYYCTIGGSLSRSSQGTLVTVSSHHH |
| | | HHHHHHH (SEQ ID NO: 308) |
| tAb0034 | CD19:HSA: | QVQLQESGPGLVKPSQTLSLTCTVSGGSISTSGMGVGWIRQHPGK |
| | CD3 | GLEWIGHIWWDDDKRYNPALKSRVTISVDTSKNQFSLKLSSVTAA |
| | | DTAVYYCARMELWSYYFDYWGQGTLVTVSSGGGGSGGGGSGGG |
| | | GSEIVLTQSPATLSLSPGERATLSCSASSSVSYMHWYQQKPGQAPR |
| | | LLIYDTSKLASGIPARFSGSGSGTDFTLTISSLEPEDFAVYYCFQGSV |
| | | YPFTFGQGTKLEIKRGGGGSGGGSEVQLVESGGGLVQPGNSLRLS |
| | | CAASGFTFSSFGMSWVRQAPGKGLEWVSSISGSGSDTLYADSVKG |
| | | RFTISRDNAKTTLYLQMNSLRPEDTAVYYCTIGGSLSRSSQGTLVT |
| | | VSSGGGGSGGGSDIVMTQSPDSLAVSLGERATINCKSSQSLLNART |
| | | GKNYLAWYQQKPGQPPKLLIYWASTRESGVPDRFSGSGSGTDFTL |
| | | TISSLQAEDVAVYYCKQSYSRRTFGGGTKVEIKGGGGSGGGGSGG |
| | | GGSGGGGSQVQLVQSGAEVKKPGASVKVSCKASGFNIKDYYMH |
| | | WVRQAPGQRLEWMGWIDLENANTIYDAKFQGRVTITRDTSASTA |
| | | YMELSSLRSEDTAVYYCARDAYGRYFYDVWGQGTLVTVSSHHH |
| | | HHHHHHH (SEQ ID NO: 309) |
| tAb0035 | CD3:CD19: | DIVMTQSPDSLAVSLGERATINCKSSQSLLNARTGKNYLAWYQQK |
| | HSA | PGQPPKLLIYWASTRESGVPDRFSGSGSGTDFTLTISSLQAEDVAV |
| | | YYCKQSYSRRTFGGGTKVEIKGGGGSGGGGSGGGGSGGGGSQVQ |
| | | LVQSGAEVKKPGASVKVSCKASGFNIKDYYMHWVRQAPGQRLE |
| | | WMGWIDLENANTIYDAKFQGRVTITRDTSASTAYMELSSLRSEDT |
| | | AVYYCARDAYGRYFYDVWGQGTLVTVSSGGGGSGGGSQVQLQE |
| | | SGPGLVKPSQTLSLTCTVSGGSISTSGMGVGWIRQHPGKGLEWIGH |
| | | IWWDDDKRYNPALKSRVTISVDTSKNQFSLKLSSVTAADTAVYYC |
| | | ARMELWSYYFDYWGQGTLVTVSSGGGGSGGGGSGGGGSEIVLTQ |
| | | SPATLSLSPGERATLSCSASSSVSYMHWYQQKPGQAPRLLIYDTSK |
| | | LASGIPARFSGSGSGTDFTLTISSLEPEDFAVYYCFQGSVYPFTFGQ |
| | | GTKLEIKRGGGGSGGGSEVQLVESGGGLVQPGNSLRLSCAASGFT |
| | | FSSFGMSWVRQAPGKGLEWVSSISGSGSDTLYADSVKGRFTISRD |
| | | NAKTTLYLQMNSLRPEDTAVYYCTIGGSLSRSSQGTLVTVSSHHH |
| | | HHHHHHH (SEQ ID NO: 310) |
| tAb0036 | CD3:HSA: | DIVMTQSPDSLAVSLGERATINCKSSQSLLNARTGKNYLAWYQQK |
| | CD19 | PGQPPKLLIYWASTRESGVPDRFSGSGSGTDFTLTISSLQAEDVAV |
| | | YYCKQSYSRRTFGGGTKVEIKGGGGSGGGGSGGGGSGGGGSQVQ |
| | | LVQSGAEVKKPGASVKVSCKASGFNIKDYYMHWVRQAPGQRLE |
| | | WMGWIDLENANTIYDAKFQGRVTITRDTSASTAYMELSSLRSEDT |
| | | AVYYCARDAYGRYFYDVWGQGTLVTVSSGGGGSGGGSEVQLVE |
| | | SGGGLVQPGNSLRLSCAASGFTFSSFGMSWVRQAPGKGLEWVSSI |
| | | SGSGSDTLYADSVKGRFTISRDNAKTTLYLQMNSLRPEDTAVYYC |
| | | TIGGSLSRSSQGTLVTVSSGGGGSGGGSQVQLQESGPGLVKPSQTL |
| | | SLTCTVSGGSISTSGMGVGWIRQHPGKGLEWIGHIWWDDDKRYN |
| | | PALKSRVTISVDTSKNQFSLKLSSVTAADTAVYYCARMELWSYYF |
| | | DYWGQGTLVTVSSGGGGSGGGGSGGGGSEIVLTQSPATLSLSPGE |
| | | RATLSCSASSSVSYMHWYQQKPGQAPRLLIYDTSKLASGIPARFSG |
| | | SGSGTDFTLTISSLEPEDFAVYYCFQGSVYPFTFGQGTKLEIKRHHH |
| | | HHHHHHH (SEQ ID NO: 311) |
| tAb0037 | HSA:CD3: | EVQLVESGGGLVQPGNSLRLSCAASGFTFSSFGMSWVRQAPGKGL |
| | CD19 | EWVSSISGSGSDTLYADSVKGRFTISRDNAKTTLYLQMNSLRPEDT |
| | | AVYYCTIGGSLSRSSQGTLVTVSSGGGGSGGGSDIVMTQSPDSLAV |
| | | SLGERATINCKSSQSLLNARTGKNYLAWYQQKPGQPPKLLIYWAS |
| | | TRESGVPDRFSGSGSGTDFTLTISSLQAEDVAVYYCKQSYSRRTFG |
| | | GGTKVEIKGGGGSGGGGSGGGGSGGGGSQVQLVQSGAEVKKPG |
| | | ASVKVSCKASGFNIKDYYMHWVRQAPGQRLEWMGWIDLENANT |
| | | IYDAKFQGRVTITRDTSASTAYMELSSLRSEDTAVYYCARDAYGR |
| | | YFYDVWGQGTLVTVSSGGGGSGGGSQVQLQESGPGLVKPSQTLS |
| | | LTCTVSGGSISTSGMGVGWIRQHPGKGLEWIGHIWWDDDKRYNP |
| | | ALKSRVTISVDTSKNQFSLKLSSVTAADTAVYYCARMELWSYYFD |
| | | YWGQGTLVTVSSGGGGSGGGGSGGGGSEIVLTQSPATLSLSPGER |
| | | ATLSCSASSSVSYMHWYQQKPGQAPRLLIYDTSKLASGIPARFSGS |
| | | GSGTDFTLTISSLEPEDFAVYYCFQGSVYPFTFGQGTKLEIKRHHH |
| | | HHHHHHH (SEQ ID NO: 312) |
| tAb0038 | HSA:CD19: | EVQLVESGGGLVQPGNSLRLSCAASGFTFSSFGMSWVRQAPGKGL |
| | CD3 | EWVSSISGSGSDTLYADSVKGRFTISRDNAKTTLYLQMNSLRPEDT |
| | | AVYYCTIGGSLSRSSQGTLVTVSSGGGGSGGGSQVQLQESGPGLV |
| | | KPSQTLSLTCTVSGGSISTSGMGVGWIRQHPGKGLEWIGHIWWDD |
| | | DKRYNPALKSRVTISVDTSKNQFSLKLSSVTAADTAVYYCARMEL |
| | | WSYYFDYWGQGTLVTVSSGGGGSGGGGSGGGGSEIVLTQSPATL |
| | | SLSPGERATLSCSASSSVSYMHWYQQKPGQAPRLLIYDTSKLASGI |
| | | PARFSGSGSGTDFTLTISSLEPEDFAVYYCFQGSVYPFTFGQGTKLE |
| | | IKRGGGGSGGGSDIVMTQSPDSLAVSLGERATINCKSSQSLLNART |
| | | GKNYLAWYQQKPGQPPKLLIYWASTRESGVPDRFSGSGSGTDFTL |
| | | TISSLQAEDVAVYYCKQSYSRRTFGGGTKVEIKGGGGSGGGGSGG |
| | | GGSGGGGSQVQLVQSGAEVKKPGASVKVSCKASGFNIKDYYMH |
| | | WVRQAPGQRLEWMGWIDLENANTIYDAKFQGRVTITRDTSASTA |
| | | YMELSSLRSEDTAVYYCARDAYGRYFYDVWGQGTLVTVSSHHH |
| | | HHHHHHH (SEQ ID NO: 313) |
| tAb0042 | CD3:HSA: | EVQLVESGGGLVQPGGSLKLSCAASGFTFNKYAINWVRQAPGKG |
| | CD19 | LEWVARIRSKYNNYATYYADQVKDRFTISRDDSKNTAYLQMNNL |
| | | KTEDTAVYYCVRHANFGNSYISYWAYWGQGTLVTVSSGGGGSG |
| | | GGGSGGGGSQTVVTQEPSLTVSPGGTVTLTCASSTGAVTSGNYPN |
| | | WVQQKPGQAPRGLIGGTKFLVPGTPARFSGSLLGGKAALTLSGVQ |
| | | PEDEAEYYCTLWYSNRWVFGGGTKLTVLGGGGSGGGSEVQLVES |
| | | GGGLVQPGNSLRLSCAASGFTFSSFGMSWVRQAPGKGLEWVSSIS |
| | | GSGSDTLYADSVKGRFTISRDNAKTTLYLQMNSLRPEDTAVYYCT |
| | | IGGSLSRSSQGTLVTVSSGGGGSGGGSQVQLQESGPGLVKPSQTLS |
| | | LTCTVSGGSISTSGMGVGWIRQHPGKGLEWIGHIWWDDDKRYNP |
| | | ALKSRVTISVDTSKNQFSLKLSSVTAADTAVYYCARMELWSYYFD |
| | | YWGQGTLVTVSSGGGGSGGGGSGGGGSEIVLTQSPATLSLSPGER |
| | | ATLSCSASSSVSYMHWYQQKPGQAPRLLIYDTSKLASGIPARFSGS |
| | | GSGTDFTLTISSLEPEDFAVYYCFQGSVYPFTFGQGTKLEIKRHHH |
| | | HHHHHHH (SEQ ID NO: 406) |

The cultures were harvested by centrifugation at 4000 rpm, and the supernatant filtered through a 0.22 mm filter. The multi-specific binding proteins, which carried a 10×His tag at the C-terminus, were purified in two steps. The first step was Nickel affinity chromatography with elution using PBS containing 400 mM imidazole. The second step was size exclusion chromatography with elution in PBS (phosphate buffered saline) pH7.2. Multi-specific binding protein concentrations were determined by UV spectroscopy, and the protein samples were concentrated when necessary. The purity of the proteins was determined by sodium dodecyl sulphate polyacrylamide gel electrophoresis (SDS-PAGE) and high performance liquid chromatography (HPLC). Specifically, HPLC was performed on an Agilent® 1100 series instrument using MabPac size exclusion column run in PBS at 0.2 mL/min. The fractions with an elution time of about 225-240 minutes were collected for further characterization.

As noted above, the constructs produced contained an anti-CD19 scFv having the amino acid sequence set forth in SEQ ID NO: 9 or 18. The binding affinity of the two CD19 binding domains to CD19 were measured by SPR using a monomeric CD19 extracellular domain and a dimeric CD19 extracellular domain fused with human IgG1 Fc. Binding kinetic parameters were measured using an instrument (available under the tradename ForteBio™ from Pall Corporation®) generally as previously described (see, Estep et al. (2013) MAbs, 5(2): 270-78). When measured with the monomeric CD19 protein, the K_{D} value of the CD19 binding domain having the sequence of SEQ ID NO: 9 was 7 nM, and the K_{D} value of the CD19 binding domain having the sequence of SEQ ID NO: 18 was 11 nM. When measured with the dimeric CD19 protein, the K_{D} value of the CD19 binding domain having the sequence of SEQ ID NO: 9 was 5 nM, and the K_{D} value of the CD19 binding domain having the sequence of SEQ ID NO: 18 was 15 nM.

### Example 2. Multi-specific Binding Proteins Induce T Cell Cytotoxicity Against CD19⁺ Target Cells

This example describes the cytotoxic activity of multi-specific binding proteins.

The T cell redirection activity of multi-specific binding proteins and BiTE® proteins were evaluated using a cell model available under the trade name KILR® Raji from Eurofins DiscoverX®. Briefly, pan T cells were isolated from primary human PBMCs from a single healthy donor by negative selection using a commercial kit (e.g. available under the name Easy Sep Human T Cell Enrichment Kit, StemCell Technologies®). T cells were maintained in RPMI 1640 medium supplemented with 10% serum and 300 IU/mL IL-2 to expand T cells. The harvested T cells were washed twice to remove any serum.

KILR® Raji cells, which expressed CD19 on the surface, were used as target cells. To opsonize the target cells, each multi-specific binding protein or BiTE® (see Table 5) was incubated with the target cells for 30 minutes at 37° C. in RPMI 1640 medium supplemented with 5% heat inactivated low IgG fetal bovine serum and penicillin-streptomycin-glutamine. The proteins were added in serial dilution at 10 different doses, with each dose run in duplicate. Human serum albumin was added to the medium of certain samples at a final concentration of 15 mg/mL. Selective proteins were also evaluated with KILR® SKOV3 cells, which were CD19-negative, as negative controls.

After opsonization, the target cells were incubated with the pan T cells at an effector-to-target (E:T) ratio of 10:1 for 6 hours at 37° C. Killing of the KILR® Raji cells resulted in release of a labeled housekeeping protein from these cells into the medium, which was quantified by addition of a KILR® detection reagent (Eurofins DiscoverX®). The luminescence signals from all wells were read on an Envision plate reader. Spontaneous release and total lysis controls were included on each plate to allow calculation of percent killing.

Percent killing was calculated from the luminescence signal values using the following formula:

$$\% killing = ( value from test protein sample - mean value from spontaneous release control ) / ( mean value from total lysis control - mean value from spontaneous release control ) \times 100. The EC 50 values were calculated from the percent killing by fitting with a dose - response curve using software available under the trade name GraphPad ® from GraphPad Software , LLC .$$

Table 6 lists the EC50 values of T cell-redirected killing in the absence and presence of human serum albumin for exemplary multi-specific binding proteins and comparator anti-CD19 BiTE® protein. No substantial killing was observed with the CD19-negative KILR® SKOV3 cells.

TABLE 6
Cytotoxic Activity of Multi-specific Binding Proteins

| | | EC50 (pg/mL) | | Fold |
|---|---|---|---|---|
| Construct | Format | −HSA | +HSA | change |
| tAb0027 | CD19:CD3:HSA | 2.33 | 67.9 | 29.1 |
| tAb0029 | CD3:CD19:HSA | 5.48 | 188.1 | 34.3 |
| tAb0030 | CD3:HSA:CD19 | 3.17 | 141.7 | 44.7 |
| tAb0031 | HSA:CD3:CD19 | 6.55 | 189.3 | 28.9 |
| tAb0032 | HSA:CD19:CD3 | 5.71 | 88.4 | 15.5 |
| tAb0033 | CD19:CD3:HSA | 20.4 | 1294 | 63.4 |
| tAb0034 | CD19:HSA:CD3 | 59.3 | 2376 | 40.1 |
| tAb0035 | CD3:CD19:HSA | 51.4 | 2184 | 42.5 |
| tAb0036 | CD3:HSA:CD19 | 103.8 | 4195 | 40.4 |
| tAb0037 | HSA:CD3:CD19 | 175.1 | 2151 | 12.3 |
| tAb0038 | HSA:CD19:CD3 | 52.6 | 288 | 5.5 |
| tAb0042 | CD3:HSA:CD19 | 69.9 | 13290 | 190.1 |
| blinatumomab | CD19:CD3 | 2854 | 10750 | 3.8 |

As shown in Table 6, the multi-specific binding proteins containing the anti-CD19 scFv having the amino acid sequence of SEQ ID NO: 9 showed stronger cytotoxic activity than those containing the anti-CD19 scFv having the amino acid sequence of SEQ ID NO: 18, regardless of the construct format, CD3 binding domain, HSA binding domain, and the presence or absence of HSA in the assay medium. From this data, it is contemplated that constructs containing this anti-CD19 scFv with the higher binding affinity to CD19 will demonstrate stronger therapeutic activity than constructs containing the other anti-CD19 scFv with the lower binding affinity.

Furthermore, all the multi-specific binding proteins tested showed lower EC₅₀ value (namely, stronger ability to induce cytotoxicity) in the absence of HSA than in the presence of HSA. Without wishing to be bound by theory, it appears that the presence of HSA causes a change in the protein complex, which was specific to the multi-specific binding proteins containing an HSA binding domain, rather than a nonspecific effect as observed with blinatumomab. The ratio of the EC₅₀ value in the presence of HSA to the EC₅₀ value in the absence of HSA, also called “fold change” herein, was used to assess the effect of HSA on the potential therapeutic activity of the multi-specific binding protein. As shown in Table 6, the construct formats with the HSA binding domain N-terminal to both the CD19 binding domain and the CD3 binding domain (namely, tAb0031, tAb0032, tAb0037, and tAb0038) showed lower fold changes than the other construct formats, regardless of which CD19 binding domain was used in the construct.

Furthermore, among the constructs having the same CD19 binding domain, CD3 binding domain, and HSA binding domain, the constructs in the CD19:CD3:HSA format (i.e., the CD19 binding domain positioned N-terminal to the CD3 binding domain, and the CD3 binding domain positioned N-terminal to the HSA binding domain), namely, tAb0027 and tAb0033, showed the lowest or second lowest EC₅₀ values both in the absence and in the presence of HSA.

### Example 3. Cytotoxicity of Multi-specific Binding proteins Against CD19⁺ Target Cells

This example provides alternative methods for determining the cytotoxic activity of a multi-specific binding protein.

The multi-specific binding proteins disclosed herein can be evaluated in in vitro assays on their mediation of T cell dependent cytotoxicity to B cell antigen positive target cells. For example, the CD19-binding multi-specific binding protein disclosed herein is evaluated in in vitro assays on its mediation of T cell dependent cytotoxicity to CD19⁺ target cells.

Fluorescence labeled CD19⁺ MEC-1 cells (a CD19⁺ human chronic B cell leukemia cell line) are incubated with isolated PBMC of random donors or CB15 T-cells (standardized T-cell line) as effector cells in the presence of the CD19-binding multi-specific binding protein. After incubation for 4 hours at 37° C. in a humidified incubator, the release of the fluorescent dye from the target cells into the supernatant is determined in a spectrofluorimeter. Target cells incubated without the CD19-binding multi-specific binding protein and target cells totally lysed by the addition of saponin at the end of the incubation serve as negative and positive controls, respectively. Based on the measured remaining living target cells, the percentage of specific cell lysis can be calculated according to the following formula: [1-(number of living targets₍ₛₐₘₚₗₑ₎/number of living targets₍ₛₚₒₙₜₐₙₑₒᵤₛ₎]×100%. Sigmoidal dose response curves and EC₅₀ values are calculated by non-linear regression/4-parameter logistic fit using software available under the trade name GraphPad® from GraphPad Software, LLC. The lysis values obtained for a given multi-specific binding protein concentration are used to calculate sigmoidal dose-response curves by 4 parameter logistic fit analysis using the Prism software. It is expected that the target cell lysis rate induced by CD19-binding multi-specific binding protein is higher than the target cell lysis rate induced by similar constructs lacking either a CD19-binding domain or a CD3-binding domain.

Alternatively, a human T-cell dependent cellular cytotoxicity (TDCC) assay is used to measure the ability of the multi-specific binding protein to direct T cells to kill tumor cells (Nazarian et al. 2015, J. Biomol. Screen, 20:519-27). In this assay, T cells and target cancer cell line cells are mixed together at a 10:1 ratio in a 384 wells plate, and varying amounts of the multi-specific binding proteins are added. After 48 hours, the T cells are washed away leaving attached to the plate target cells that were not killed by the T cells. To quantitate the remaining viable cells, a luminescent cell viability assay (available under the tradename CellTiter-GLO® from Promega®) is used. It is contemplated that the killing rate of B-cell antigen expressing cancer cell induced by CD19-binding multi-specific binding protein will be higher than that induced by similar constructs lacking either a CD19-binding domain or a CD3-binding domain and/or other negative control molecules.

### Example 4. Pharmacokinetics of Multi-specific Binding Proteins with HSA Binding Domain

This example is designed to determine the pharmacokinetics of multi-specific binding proteins.

Multi-specific binding proteins containing a domain that binds CD19, a domain that binds CD3, and a domain that binds serum albumin are tested in the cynomolgus monkey in the context of pharmacokinetic (PK) studies to evaluate the serum elimination time of the multi-specific binding protein.

The multi-specific binding proteins are administered as intravenous bolus or intravenous infusion. The multi-specific binding proteins are administered in a dose-linear, pharmacokinetic relevant range of 0.5 μg/kg to 3 μg/kg, 6 μg/kg, 12 μg/kg, and 15 μg/kg, respectively. For purposes of comparability, the serum concentrations of the multi-specific binding proteins are does-normalized and molecular weight-normalized (described in nmol).

For each multi-specific binding protein, a group of at least two to three animals are used. Blood samples are collected and serum is prepared for determination of serum concentrations of the multi-specific binding proteins. Serum multi-specific binding protein levels are measured using an immunoassay. The assay is performed by capturing the multi-specific binding protein via its CD19-binding domain, while an antibody directed against the CD3-binding domain of the multi-specific binding protein is used for detection. The serum concentration-time profiles are used to determine PK parameters using known analytical methods such as those described in Ritschel W A and Kearns G L, 1999, IN: Handbook Of Basic Pharmacokinetics Including Clinical Applications, 5th edition, American Pharmaceutical Assoc., Washington, D.C. and software available under the trade name WinNonlin® professional V. 3.1 from Certara, L.P.).

Alternatively, the serum half-life of the various multi-specific binding proteins containing the serum albumin binding domain is compared to that of control constructs capable of binding CD19 and CD3 but lacking a serum albumin binding domain by including in the experiment another cynomolgus monkey group that receives the control constructs. Additional domains can be included such that the control constructs are similar in size to the multi-specific binding proteins.

It is expected that CD19-binding multi-specific binding protein will have significantly longer serum half-life compared to similar constructs capable of binding CD19 and CD3 but lacking a serum albumin binding domain and/or other negative control molecules.

### Example 5. Determination of Antigen Affinity by Flow Cytometry

This example is designed to determine the affinity of a multi-specific binding protein to an antigen.

Various multi-specific binding proteins disclosed herein are tested for their binding affinities to human CD3⁺ cells and the corresponding B cell surface antigen positive cells, such as human CD19⁺ cells. The multi-specific binding proteins are also tested for their binding affinities to cynomolgus CD3⁺ cells and the corresponding B cell surface antigen positive cells, such as cynomolgus CD19⁺ cells.

CD3⁺ and CD19⁺ cells are incubated with 100 μL of serial dilutions of the multi-specific binding protein. After washing three times with FACS buffer the cells are incubated with 0.1 mL of 10 μg/mL mouse monoclonal anti-idiotype antibody in the same buffer for 45 mins on ice. After a second washing cycle, the cells are incubated with 0.1 mL of 15 μg/mL FITC-conjugated goat anti-mouse IgG antibodies under the same conditions as before. As a control, cells are incubated with the anti-His IgG followed by the FITC-conjugated goat anti-mouse IgG antibodies without the multi-specific binding protein. The cells are then washed again and resuspended in 0.2 mL of FACS buffer containing 2 μg/mL propidium iodide (PI) in order to exclude dead cells. The fluorescence of 1×10⁴ living cells is measured using a commercially available flow cytometer and software. Mean fluorescence intensities of the cell samples are calculated using software available under the trade names CXP software (Beckman-Coulter®, Krefeld, Germany) or Incyte™ software (Merck Millipore, Schwalbach, Germany). K_{D} values for one-site binding can be calculated using normalized fluorescence intensity values with known computational equations such as those supplied under the trade name GraphPad Prism® software from GraphPad® Software, La Jolla Calif. USA. CD3 binding affinity and cross-reactivity are evaluated in titration and flow cytometric experiments on CD3⁺ Jurkat cells and the cynomolgus CD3⁺ HSC-F cell line. CD19 binding and cross-reactivity are assessed on the human CD19⁺ tumor cell lines. The K_{D} ratio of cross-reactivity can be calculated using the K_{D} values determined on the CHO cell lines expressing either recombinant human or recombinant cynomolgus antigens.

### Example 6. Cytokine Production Induced by Multi-Specific Binding Proteins

This example is designed to determine the ability of a multi-specific binding protein to induce cytokine production from immune cells.

Assays available under the trade name AlphaLISA® (Perkin Elmer®) for TNFα and Interferon γ are used to obtain evidence that T cells are activated by the multi-specific binding proteins of current invention, such as CD19-binding multi-specific binding protein, in the presence of target cells, such as CD19⁺ B cells. For this assay, primary human T cells and human tumor cells expressing B cell surface antigen are incubated in the presence of the CD19-binding multi-specific binding protein as described under cytotoxicity assays. After 48 hours of incubation, 2 microliter aliquots of the assay supernatants are analyzed according to the manufacturer's instructions. It is contemplated that the TNFα or Interferon γ level induced by CD19-binding multi-specific binding protein is higher than that induced by similar constructs lacking either a CD19-binding domain or a CD3-binding domain and/or other negative control molecules.

### Example 7. Identification of scFv Variants that Bind Human CD3ε

This example is designed to identify variants of the antigen-binding sites disclosed herein that bind human CD3ε.

The binding properties of the parental CD3ε binding construct to biotin-CD3ε and to biotin-HSA are characterized. To construct the anti-CD3ε scFv phage libraries, a single substitution library is provided for the heavy chain CDR1, heavy chain CDR2, heavy chain CDR3, light chain CDR1, light chain CDR2, and light chain CDR3 domains. Residues are varied one at a time via mutagenesis. For selection of clones and determination of binding affinity, single substitution libraries are bound to biotinylated human CD3E, washed, eluted, and counted. Biotinylated cynomolgus CD3ε is used as the round 1 selection target, and washed for 4 hours after combinatorial phage binding from the two independent libraries (˜2× selection). Biotinylated human CD3ε is used as the round 2 selection target, and washed for 3 hours after binding of both libraries (<2× selection). PCRed inserts from the second round of selection are sub-cloned into the pcDNA3.4 His6 expression vector. 180 clones are picked and DNA is purified, sequenced, and transfected into an expression system available under the trade name Expi293® from Thermo Fisher®. A panel of sixteen clones with a range of affinities for human CD3ε are selected for more precise determination of the parameters such as the dissociation constant (K_{D}), the dissociation rate (k_{d} or k_{off}), and the association rate (kₐ or kₒₙ).

### INCORPORATION BY REFERENCE

All publications and patents cited throughout the text of this specification (including all patents, patent applications, scientific publications, manufacturer's specifications, instructions, etc.), whether supra or infra, are hereby incorporated by reference in their entirety for all purposes. To the extent the material incorporated by reference contradicts or is inconsistent with this specification, the specification will supersede any such material.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A multi-specific binding protein comprising:
(a) a first antigen-binding site that binds to human CD19;
(b) a second antigen-binding site that binds to human CD3;
(c) a third antigen-binding site that binds to human serum albumin (HSA);
and wherein the multi-specific binding protein further comprises a sequence selected from the group consisting of SEQ ID NO: 303, 304, 305, 306, 307, 308, 309, 310, 311, 312, 313, and 406.

2. The multi-specific binding protein of claim 1, wherein the first antigen-binding site binds to human CD19 with a dissociation constant (K_{D}) equal to or lower than 20 nM.

3. The multi-specific binding protein of claim 1, wherein the first antigen-binding site has a melting temperature of at least 60° C.

4. The multi-specific binding protein of claim 1, wherein the second antigen-binding site binds to human CD3 with a K_{D} equal to or lower than 10 nM.

5. The multi-specific binding protein of claim 1, wherein the second antigen-binding site has a melting temperature of at least 60° C.

6. The multi-specific binding protein of claim 1, wherein the third antigen-binding site binds to HSA with a K_{D} equal to or lower than 20 nM.

7. The multi-specific binding protein of claim 1, wherein the third antigen-binding site has a melting temperature of at least 60° C.

8. The multi-specific binding protein of claim 1, wherein the serum half-life of the multi-specific binding protein is at least 24 hours.

9. A pharmaceutical composition comprising the multi-specific binding protein of claim 1 and a pharmaceutically acceptable carrier.

10. An isolated polynucleotide encoding the multi-specific binding protein of claim 1.

11. A vector comprising the polynucleotide of claim 10.

12. A isolated recombinant host cell comprising the polynucleotide of claim 10.

13. A method of producing a multi-specific binding protein, the method comprising culturing the host cell of claim 12 under suitable conditions that allow expression of the multi-specific binding protein.

14. A complex comprising a T cell expressing CD3, a B cell expressing CD19, and the multi-specific binding protein of claim 1, wherein the multi-specific binding protein is capable of simultaneously binding to both the T cell and the B cell.


SEQUENCE LISTING (407 sequences)

SEQ ID NO: 1 - protein, 121 aa, synthetic construct
  REGION 1..121: note = Description of Artificial Sequence: Synthetic polypeptide
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSS

SEQ ID NO: 2 - protein, 112 aa, synthetic construct
  REGION 1..112: note = Description of Artificial Sequence: Synthetic polypeptide
DIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIK

SEQ ID NO: 3 - protein, 5 aa, synthetic construct
  REGION 1..5: note = Description of Artificial Sequence: Synthetic peptide
DYIMH

SEQ ID NO: 4 - protein, 17 aa, synthetic construct
  REGION 1..17: note = Description of Artificial Sequence: Synthetic peptide
YINPYNDGSKYTEKFQG

SEQ ID NO: 5 - protein, 12 aa, synthetic construct
  REGION 1..12: note = Description of Artificial Sequence: Synthetic peptide
GTYYYGPQLFDY

SEQ ID NO: 6 - protein, 16 aa, synthetic construct
  REGION 1..16: note = Description of Artificial Sequence: Synthetic peptide
KSSQSLETSTGTTYLN

SEQ ID NO: 7 - protein, 7 aa, synthetic construct
  REGION 1..7: note = Description of Artificial Sequence: Synthetic peptide
RVSKRFS

SEQ ID NO: 8 - protein, 9 aa, synthetic construct
  REGION 1..9: note = Description of Artificial Sequence: Synthetic peptide
LQLLEDPYT

SEQ ID NO: 9 - protein, 248 aa, synthetic construct
  REGION 1..248: note = Description of Artificial Sequence: Synthetic polypeptide
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSGGGGSGGGGSGGGGSDIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIK

SEQ ID NO: 10 - protein, 120 aa, synthetic construct
  REGION 1..120: note = Description of Artificial Sequence: Synthetic polypeptide
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSS

SEQ ID NO: 11 - protein, 107 aa, synthetic construct
  REGION 1..107: note = Description of Artificial Sequence: Synthetic polypeptide
EIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKR

SEQ ID NO: 12 - protein, 7 aa, synthetic construct
  REGION 1..7: note = Description of Artificial Sequence: Synthetic peptide
TSGMGVG

SEQ ID NO: 13 - protein, 16 aa, synthetic construct
  REGION 1..16: note = Description of Artificial Sequence: Synthetic peptide
HIWWDDDKRYNPALKS

SEQ ID NO: 14 - protein, 10 aa, synthetic construct
  REGION 1..10: note = Description of Artificial Sequence: Synthetic peptide
MELWSYYFDY

SEQ ID NO: 15 - protein, 10 aa, synthetic construct
  REGION 1..10: note = Description of Artificial Sequence: Synthetic peptide
SASSSVSYMH

SEQ ID NO: 16 - protein, 7 aa, synthetic construct
  REGION 1..7: note = Description of Artificial Sequence: Synthetic peptide
DTSKLAS

SEQ ID NO: 17 - protein, 9 aa, synthetic construct
  REGION 1..9: note = Description of Artificial Sequence: Synthetic peptide
FQGSVYPFT

SEQ ID NO: 18 - protein, 242 aa, synthetic construct
  REGION 1..242: note = Description of Artificial Sequence: Synthetic polypeptide
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKR

SEQ ID NO: 19 - protein, 121 aa, synthetic construct
  REGION 1..121: note = Description of Artificial Sequence: Synthetic polypeptide
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 20 - protein, 112 aa, synthetic construct
  REGION 1..112: note = Description of Artificial Sequence: Synthetic polypeptide
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 21 - protein, 5 aa, synthetic construct
  REGION 1..5: note = Description of Artificial Sequence: Synthetic peptide
SYVMH

SEQ ID NO: 22 - protein, 14 aa, synthetic construct
  REGION 1..14: note = Description of Artificial Sequence: Synthetic peptide
WIGYINPYNDGTKY

SEQ ID NO: 23 - protein, 12 aa, synthetic construct
  REGION 1..12: note = Description of Artificial Sequence: Synthetic peptide
GTYYYGTRVFDY

SEQ ID NO: 24 - protein, 16 aa, synthetic construct
  REGION 1..16: note = Description of Artificial Sequence: Synthetic peptide
RSSKSLQNVNGNTYLY

SEQ ID NO: 25 - protein, 7 aa, synthetic construct
  REGION 1..7: note = Description of Artificial Sequence: Synthetic peptide
RMSNLNS

SEQ ID NO: 26 - protein, 9 aa, synthetic construct
  REGION 1..9: note = Description of Artificial Sequence: Synthetic peptide
MQHLEYPIT

SEQ ID NO: 27 - protein, 124 aa, synthetic construct
  REGION 1..124: note = Description of Artificial Sequence: Synthetic polypeptide
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTSVTVSS

SEQ ID NO: 28 - protein, 111 aa, synthetic construct
  REGION 1..111: note = Description of Artificial Sequence: Synthetic polypeptide
DILLTQTPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 29 - protein, 5 aa, synthetic construct
  REGION 1..5: note = Description of Artificial Sequence: Synthetic peptide
SYWMN

SEQ ID NO: 30 - protein, 17 aa, synthetic construct
  REGION 1..17: note = Description of Artificial Sequence: Synthetic peptide
QIWPGDGDTNYNGKFKG

SEQ ID NO: 31 - protein, 15 aa, synthetic construct
  REGION 1..15: note = Description of Artificial Sequence: Synthetic peptide
RETTTVGRYYYAMDY

SEQ ID NO: 32 - protein, 15 aa, synthetic construct
  REGION 1..15: note = Description of Artificial Sequence: Synthetic peptide
KASQSVDYDGDSYLN

SEQ ID NO: 33 - protein, 7 aa, synthetic construct
  REGION 1..7: note = Description of Artificial Sequence: Synthetic peptide
DASNLVS

SEQ ID NO: 34 - protein, 9 aa, synthetic construct
  REGION 1..9: note = Description of Artificial Sequence: Synthetic peptide
QQSTEDPWT

SEQ ID NO: 35 - protein, 124 aa, synthetic construct
  REGION 1..124: note = Description of Artificial Sequence: Synthetic polypeptide
QVQLQQSGAEVKKPGSSVKVSCKASGYAFSSYWMNWVRQRPGQGLEWIGQIWPGDGDTNYNGKFKGRATITADESTNTAYMELSSLRSEDTAFYSCARRETTTVGRYYYAMDYWGQGTTVTVSS

SEQ ID NO: 36 - protein, 112 aa, synthetic construct
  REGION 1..112: note = Description of Artificial Sequence: Synthetic polypeptide
DIQLTQSPSSLSASVGDRVTITCKASQSVDYDGDSYLNWYQQIPGKAPKLLIYDASNLVSGIPPRFSGSGSGTDYTFTISSLQPEDIATYHCQQSTEDPWTFGGGTKLQIKR

SEQ ID NO: 37 - protein, 120 aa, synthetic construct
  REGION 1..120: note = Description of Artificial Sequence: Synthetic polypeptide
QVQLEQPGAEVVKPGASVKVSCKTSGYTFTSNWMHWVKQTPGKGLEWIGEIDPSDSYTNYNQKFDGKAKLTVDKSSSTAYMEVSDLTAEDSATYYCARGSNPYYYAMDYWGQGTSVTVSS

SEQ ID NO: 38 - protein, 104 aa, synthetic construct
  REGION 1..104: note = Description of Artificial Sequence: Synthetic polypeptide
QIVLTQSPATLSASPGEKATMTCSASSGVNYMHWYQQKPGTSPKRWIYDTDKTASGVPARFSGSGSGTSYSLTISSMEAEDAATYYCHQRGSYTFGGGTKLEIK

SEQ ID NO: 39 - protein, 5 aa, synthetic construct
  REGION 1..5: note = Description of Artificial Sequence: Synthetic peptide
SNWMH

SEQ ID NO: 40 - protein, 10 aa, synthetic construct
  REGION 1..10: note = Description of Artificial Sequence: Synthetic peptide
EIDPSDSYTN

SEQ ID NO: 41 - protein, 11 aa, synthetic construct
  REGION 1..11: note = Description of Artificial Sequence: Synthetic peptide
GSNPYYYAMDY

SEQ ID NO: 42 - protein, 10 aa, synthetic construct
  REGION 1..10: note = Description of Artificial Sequence: Synthetic peptide
SASSGVNYMH

SEQ ID NO: 43 - protein, 7 aa, synthetic construct
  REGION 1..7: note = Description of Artificial Sequence: Synthetic peptide
DTDKTAS

SEQ ID NO: 44 - protein, 7 aa, synthetic construct
  REGION 1..7: note = Description of Artificial Sequence: Synthetic peptide
HQRGSYT

SEQ ID NO: 45 - protein, 121 aa, synthetic construct
  REGION 1..121: note = Description of Artificial Sequence: Synthetic polypeptide
EVQLVQSGAEVKKPGESLKISCKGSGYSFSSSWIGWVRQMPGKGLEWMGIIYPDDSDTRYSPSFQGQVTISADKSIRTAYLQWSSLKASDTAMYYCARHVTMIWGVIIDFWGQGTLVTVSS

SEQ ID NO: 46 - protein, 107 aa, synthetic construct
  REGION 1..107: note = Description of Artificial Sequence: Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIK

SEQ ID NO: 47 - protein, 5 aa, synthetic construct
  REGION 1..5: note = Description of Artificial Sequence: Synthetic peptide
SSWIG

SEQ ID NO: 48 - protein, 17 aa, synthetic construct
  REGION 1..17: note = Description of Artificial Sequence: Synthetic peptide
IIYPDDSDTRYSPSFQG

SEQ ID NO: 49 - protein, 12 aa, synthetic construct
  REGION 1..12: note = Description of Artificial Sequence: Synthetic peptide
HVTMIWGVIIDF

SEQ ID NO: 50 - protein, 11 aa, synthetic construct
  REGION 1..11: note = Description of Artificial Sequence: Synthetic peptide
RASQGISSALA

SEQ ID NO: 51 - protein, 7 aa, synthetic construct
  REGION 1..7: note = Description of Artificial Sequence: Synthetic peptide
DASSLES

SEQ ID NO: 52 - protein, 9 aa, synthetic construct
  REGION 1..9: note = Description of Artificial Sequence: Synthetic peptide
QQFNSYPFT

SEQ ID NO: 53 - protein, 107 aa, synthetic construct
  REGION 1..107: note = Description of Artificial Sequence: Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 54 - protein, 9 aa, synthetic construct
  REGION 1..9: note = Description of Artificial Sequence: Synthetic peptide
QQFNSYPYT

SEQ ID NO: 55 - protein, 119 aa, synthetic construct
  REGION 1..119: note = Description of Artificial Sequence: Synthetic polypeptide
QVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 56 - protein, 108 aa, synthetic construct
  REGION 1..108: note = Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIK

SEQ ID NO: 57 - protein, 5 aa, synthetic construct
  REGION 1..5: note = Description of Artificial Sequence: Synthetic peptide
SYAIS

SEQ ID NO: 58 - protein, 17 aa, synthetic construct
  REGION 1..17: note = Description of Artificial Sequence: Synthetic peptide
GIIPIFGTTNYAQQFQG

SEQ ID NO: 59 - protein, 10 aa, synthetic construct
  REGION 1..10: note = Description of Artificial Sequence: Synthetic peptide
EAVAADWLDP

SEQ ID NO: 60 - protein, 12 aa, synthetic construct
  REGION 1..12: note = Description of Artificial Sequence: Synthetic peptide
RASQSVSSSYLA

SEQ ID NO: 61 - protein, 7 aa, synthetic construct
  REGION 1..7: note = Description of Artificial Sequence: Synthetic peptide
GASSRAT

SEQ ID NO: 62 - protein, 9 aa, synthetic construct
  REGION 1..9: note = Description of Artificial Sequence: Synthetic peptide
QQYGSSRFT

SEQ ID NO: 63 - protein, 124 aa, synthetic construct
  REGION 1..124: note = Description of Artificial Sequence: Synthetic polypeptide
EVQLVQSGAEVKKPGESLKISCKGSGYSFTSYWIAWVRQMPGKGLEWMGIIYPGDSDTRYSPSFQGQVTISADKSISTAYLQWSSLKASDTAMYYCARQGYSSGWDSYYGMGVWGQGTTVTVSS

SEQ ID NO: 64 - protein, 107 aa, synthetic construct; identical to SEQ ID NO: 53
  REGION 1..107: note = Description of Artificial Sequence: Synthetic polypeptide

SEQ ID NO: 65 - protein, 5 aa, synthetic construct
  REGION 1..5: note = Description of Artificial Sequence: Synthetic peptide
SYWIA

SEQ ID NO: 66 - protein, 17 aa, synthetic construct
  REGION 1..17: note = Description of Artificial Sequence: Synthetic peptide
IIYPGDSDTRYSPSFQG

SEQ ID NO: 67 - protein, 15 aa, synthetic construct
  REGION 1..15: note = Description of Artificial Sequence: Synthetic peptide
QGYSSGWDSYYGMGV

SEQ ID NO: 68 - protein, 123 aa, synthetic construct
  REGION 1..123: note = Description of Artificial Sequence: Synthetic polypeptide
QVQLVQSGAEVKKPGSSVKVSCKASGGTFSSYTINWVRQAPGQGLEWMGGIIPIFGIPNYAQKFQGRVTITADESTNTAYMELSSLRAEDTAVYYCARASGGSADYSYGMDVWGQGTAVTVSS

SEQ ID NO: 69 - protein, 107 aa, synthetic construct
  REGION 1..107: note = Description of Artificial Sequence: Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYKRYPYTFGQGTKLEIK

SEQ ID NO: 70 - protein, 5 aa, synthetic construct
  REGION 1..5: note = Description of Artificial Sequence: Synthetic peptide
SYTIN

SEQ ID NO: 71 - protein, 17 aa, synthetic construct
  REGION 1..17: note = Description of Artificial Sequence: Synthetic peptide
GIIPIFGIPNYAQKFQG

SEQ ID NO: 72 - protein, 14 aa, synthetic construct
  REGION 1..14: note = Description of Artificial Sequence: Synthetic peptide
ASGGSADYSYGMDV

SEQ ID NO: 73 - protein, 11 aa, synthetic construct
  REGION 1..11: note = Description of Artificial Sequence: Synthetic peptide
RASQGISSWLA

SEQ ID NO: 74 - protein, 7 aa, synthetic construct
  REGION 1..7: note = Description of Artificial Sequence: Synthetic peptide
AASSLQS

SEQ ID NO: 75 - protein, 9 aa, synthetic construct
  REGION 1..9: note = Description of Artificial Sequence: Synthetic peptide
QQYKRYPYT

SEQ ID NO: 76 - protein, 121 aa, synthetic construct
  REGION 1..121: note = Description of Artificial Sequence: Synthetic polypeptide
EVQLVQSGAEVKKPGESLNISCKGSGYSFTSYWIGWVRQMPGKGLEWMGIIYPGDSDTRYSPSFQGQVTISADKSINTAYLQWSSLKASDTAMYYCARGVSMIWGVIMDVWGQGTTVTVSS

SEQ ID NO: 77 - protein, 107 aa, synthetic construct
  REGION 1..107: note = Description of Artificial Sequence: Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYPWTFGQGTKVEIK

SEQ ID NO: 78 - protein, 5 aa, synthetic construct
  REGION 1..5: note = Description of Artificial Sequence: Synthetic peptide
SYWIG

SEQ ID NO: 79 - protein, 12 aa, synthetic construct
  REGION 1..12: note = Description of Artificial Sequence: Synthetic peptide
GVSMIWGVIMDV

SEQ ID NO: 80 - protein, 9 aa, synthetic construct
  REGION 1..9: note = Description of Artificial Sequence: Synthetic peptide
QQFNSYPWT

SEQ ID NO: 81 - protein, 124 aa, synthetic construct
  REGION 1..124: note = Description of Artificial Sequence: Synthetic polypeptide
EVQLVQSGAEVKKPGESLQISCKGSGYTFTNYWIAWVRQMPGKGLEWMGIIYPGDSDTRYSPSFQGQVTISADKSISTAYLQWSGLKASDTAMYYCARQGYSSGWRSYYGMGVWGQGTTVTVSS

SEQ ID NO: 82 - protein, 107 aa, synthetic construct
  REGION 1..107: note = Description of Artificial Sequence: Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYPHTFGQGTKLEIK

SEQ ID NO: 83 - protein, 5 aa, synthetic construct
  REGION 1..5: note = Description of Artificial Sequence: Synthetic peptide
NYWIA

SEQ ID NO: 84 - protein, 15 aa, synthetic construct
  REGION 1..15: note = Description of Artificial Sequence: Synthetic peptide
QGYSSGWRSYYGMGV

SEQ ID NO: 85 - protein, 9 aa, synthetic construct
  REGION 1..9: note = Description of Artificial Sequence: Synthetic peptide
QQFNSYPHT

SEQ ID NO: 86 - protein, 122 aa, synthetic construct
  REGION 1..122: note = Description of Artificial Sequence: Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGVSGIYNLHGFDIWGQGTLVTVSS

SEQ ID NO: 87 - protein, 108 aa, synthetic construct
  REGION 1..108: note = Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQGRFGSPFTFGQGTKVEIK

SEQ ID NO: 88 - protein, 8 aa, synthetic construct
  REGION 1..8: note = Description of Artificial Sequence: Synthetic peptide
GYSFTSYW

SEQ ID NO: 89 - protein, 8 aa, synthetic construct
  REGION 1..8: note = Description of Artificial Sequence: Synthetic peptide
IYPGDSDT

SEQ ID NO: 90 - protein, 15 aa, synthetic construct
  REGION 1..15: note = Description of Artificial Sequence: Synthetic peptide
ARGVSGIYNLHGFDI

SEQ ID NO: 91 - protein, 6 aa, synthetic construct
  REGION 1..6: note = Description of Artificial Sequence: Synthetic peptide
QSISSY

SEQ ID NO: 92 - skipped

SEQ ID NO: 93 - protein, 10 aa, synthetic construct
  REGION 1..10: note = Description of Artificial Sequence: Synthetic peptide
QQGRFGSPFT

SEQ ID NO: 94 - protein, 117 aa, synthetic construct
  REGION 1..117: note = Description of Artificial Sequence: Synthetic polypeptide
QVQLVETGGGLVQPGGSLRLSCAASGFTFSSYAMSWVRQAPGKGLEWVSAISGSGGSTYYADSVKGRFTISRDNSKNTLYLQMNSLRAEDTAVYYCARYYYSRLDYWGQGTLVTVSS

SEQ ID NO: 95 - protein, 113 aa, synthetic construct
  REGION 1..113: note = Description of Artificial Sequence: Synthetic polypeptide
QTVVTQEPSVSAAPGQKVTISCSGSSSNIGNNYVSWYQQLPGTAPKLLIYDNNKRPSGIPDRFSGSKSGTSATLGITGLQTGDEADYYCGTWDSSLSAGVFGTGTKLTVLGSR

SEQ ID NO: 96 - protein, 249 aa, synthetic construct
  REGION 1..249: note = Description of Artificial Sequence: Synthetic polypeptide
QTVVTQEPSVSAAPGQKVTISCSGSSSNIGNNYVSWYQQLPGTAPKLLIYDNNKRPSGIPDRFSGSKSGTSATLGITGLQTGDEADYYCGTWDSSLSAGVFGTGTKLTVLGSRGGGGSGGGGSGGGGSLEMAQVQLVETGGGLVQPGGSLRLSCAASGFTFSSYAMSWVRQAPGKGLEWVSAISGSGGSTYYADSVKGRFTISRDNSKNTLYLQMNSLRAEDTAVYYCARYYYSRLDYWGQGTLVTVSS

SEQ ID NO: 97 - protein, 119 aa, synthetic construct
  REGION 1..119: note = Description of Artificial Sequence: Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSS

SEQ ID NO: 98 - protein, 112 aa, synthetic construct
  REGION 1..112: note = Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIK

SEQ ID NO: 99 - protein, 9 aa, synthetic construct
  REGION 1..9: note = Description of Artificial Sequence: Synthetic peptide
FNIKDYYMH

SEQ ID NO: 100 - protein, 17 aa, synthetic construct
  REGION 1..17: note = Description of Artificial Sequence: Synthetic peptide
WIDLENANTIYDAKFQG

SEQ ID NO: 101 - protein, 12 aa, synthetic construct
  REGION 1..12: note = Description of Artificial Sequence: Synthetic peptide
ARDAYGRYFYDV

SEQ ID NO: 102 - protein, 17 aa, synthetic construct
  REGION 1..17: note = Description of Artificial Sequence: Synthetic peptide
KSSQSLLNARTGKNYLA

SEQ ID NO: 103 - protein, 7 aa, synthetic construct
  REGION 1..7: note = Description of Artificial Sequence: Synthetic peptide
WASTRES

SEQ ID NO: 104 - protein, 8 aa, synthetic construct
  REGION 1..8: note = Description of Artificial Sequence: Synthetic peptide
KQSYSRRT

SEQ ID NO: 105 - protein, 251 aa, synthetic construct
  REGION 1..251: note = Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSS

SEQ ID NO: 106 - protein, 125 aa, synthetic construct
  REGION 1..125: note = Description of Artificial Sequence: Synthetic polypeptide
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSS

SEQ ID NO: 107 - protein, 109 aa, synthetic construct
  REGION 1..109: note = Description of Artificial Sequence: Synthetic polypeptide
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 108 - protein, 10 aa, synthetic construct
  REGION 1..10: note = Description of Artificial Sequence: Synthetic peptide
GFTFNKYAIN

SEQ ID NO: 109 - protein, 18 aa, synthetic construct
  REGION 1..18: note = Description of Artificial Sequence: Synthetic peptide
RIRSKYNNYATYYADQVK

SEQ ID NO: 110 - protein, 14 aa, synthetic construct
  REGION 1..14: note = Description of Artificial Sequence: Synthetic peptide
HANFGNSYISYWAY

SEQ ID NO: 111 - protein, 14 aa, synthetic construct
  REGION 1..14: note = Description of Artificial Sequence: Synthetic peptide
ASSTGAVTSGNYPN

SEQ ID NO: 112 - protein, 7 aa, synthetic construct
  REGION 1..7: note = Description of Artificial Sequence: Synthetic peptide
GTKFLVP

SEQ ID NO: 113 - protein, 9 aa, synthetic construct
  REGION 1..9: note = Description of Artificial Sequence: Synthetic peptide
TLWYSNRWV

SEQ ID NO: 114 - protein, 249 aa, synthetic construct
  REGION 1..249: note = Description of Artificial Sequence: Synthetic polypeptide
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 115 - protein, 119 aa, synthetic construct
  REGION 1..119: note = Description of Artificial Sequence: Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDSYGRYFYDVWGQGTLVTVSS

SEQ ID NO: 116 - protein, 112 aa, synthetic construct
  REGION 1..112: note = Description of Artificial Sequence: Synthetic polypeptide
DIVMTQSPDSLAVSLGERATINCKSSQSLLNSRTGKNYLAWYQQKPGQPPKLLIYWASTRESGVPDRFSGSGSGTDFTLTISSLQAEDVAVYYCKQSYSRRTFGGGTKVEIK

SEQ ID NO: 117 - protein, 119 aa, synthetic construct
  REGION 1..119: note = Description of Artificial Sequence: Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDVYGRYFYDLWGQGTLVTVSS

SEQ ID NO: 118 - protein, 112 aa, synthetic construct
  REGION 1..112: note = Description of Artificial Sequence: Synthetic polypeptide
DIVMTQSPDSLAVSLGERATINCKSSQSLLESRTGKNYLAWYQQKPGQPPKLLIYWASTRESGVPDRFSGSGSGTDFTLTISSLQAEDVAVYYCKQSYSRRTFGGGTKVEIK

SEQ ID NO: 119 - protein, 121 aa, synthetic construct
  REGION 1..121: note = Description of Artificial Sequence: Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVE

SEQ ID NO: 120 - protein, 108 aa, synthetic construct
  REGION 1..108: note = Description of Artificial Sequence: Synthetic polypeptide
VDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 121 - protein, 115 aa, synthetic construct [sequence omitted]

SEQ ID NO: 122 - protein, 5 aa, synthetic construct
  REGION 1..5: note = Description of Artificial Sequence: Synthetic peptide
SFGMS

SEQ ID NO: 123 - protein, 10 aa, synthetic construct
  REGION 1..10: note = Description of Artificial Sequence: Synthetic peptide
GFTFSSFGMS

SEQ ID NO: 124 - protein, 10 aa, synthetic construct
  REGION 1..10: note = Description of Artificial Sequence: Synthetic peptide
SISGSGSDTL

SEQ ID NO: 125 - protein, 17 aa, synthetic construct
  REGION 1..17: note = Description of Artificial Sequence: Synthetic peptide
SISGSGSDTLYADSVKG

SEQ ID NO: 126 - protein, 6 aa, synthetic construct
  REGION 1..6: note = Description of Artificial Sequence: Synthetic peptide
GGSLSR

SEQ ID NO: 127 - protein, 115 aa, synthetic construct [sequence omitted]

SEQ ID NO: 128 - protein, 10 aa, synthetic construct
  REGION 1..10: note = Description of Artificial Sequence: Synthetic peptide
GFTFRSFGMS

SEQ ID NO: 129 - protein, 115 aa, synthetic construct [sequence omitted]

SEQ ID NO: 130 - protein, 115 aa, synthetic construct [sequence omitted]

SEQ ID NO: 131 - protein, 115 aa, synthetic construct [sequence omitted]

SEQ ID NO: 132 - protein, 115 aa, synthetic construct [sequence omitted]

SEQ ID NO: 133 - protein, 115 aa, synthetic construct [sequence omitted]

SEQ ID NO: 134 - protein, 115 aa, synthetic construct [sequence omitted]

SEQ ID NO: 135 - protein, 115 aa, synthetic construct [sequence omitted]

SEQ ID NO: 136 - protein, 115 aa, synthetic construct [sequence omitted]

SEQ ID NO: 137 - protein, 116 aa, synthetic construct
  REGION 1..116: note = Description of Artificial Sequence: Synthetic polypeptide
AVQLVDSGGGLVQPGGSLRLSCAASGFSFGSFGMSWVRQYPGKEPEWVSSINGRGDDTRYADSVKGRFSISRDNAKNTLYLQMNSLKPEDTAEYYCTIGRSVSRSRTQGTQVTVSS

SEQ ID NO: 138 - protein, 10 aa, synthetic construct
  REGION 1..10: note = Description of Artificial Sequence: Synthetic peptide
GFSFGSFGMS

SEQ ID NO: 139 - protein, 10 aa, synthetic construct
  REGION 1..10: note = Description of Artificial Sequence: Synthetic peptide
SINGRGDDTR

SEQ ID NO: 140 - protein, 17 aa, synthetic construct
  REGION 1..17: note = Description of Artificial Sequence: Synthetic peptide
SINGRGDDTRYADSVKG

SEQ ID NO: 141 - protein, 7 aa, synthetic construct
  REGION 1..7: note = Description of Artificial Sequence: Synthetic peptide
GRSVSRS

SEQ ID NO: 142 - protein, 114 aa, synthetic construct
  REGION 1..114: note = Description of Artificial Sequence: Synthetic polypeptide
AVQLVESGGGLVQPGGSLRLTCTASGFTFRSFGMSWVRQAPGKDQEWVSAISADSSTKNYADSVKGRFTISRDNAKKMLYLEMNSLKPEDTAVYYCVIGRGSPSSPGTQVTVSS

SEQ ID NO: 143 - protein, 10 aa, synthetic construct
  REGION 1..10: note = Description of Artificial Sequence: Synthetic peptide
AISADSSTKN

SEQ ID NO: 144 - protein, 17 aa, synthetic construct
  REGION 1..17: note = Description of Artificial Sequence: Synthetic peptide
AISADSSTKNYADSVKG

SEQ ID NO: 145 - protein, 5 aa, synthetic construct
  REGION 1..5: note = Description of Artificial Sequence: Synthetic peptide
GRGSP

SEQ ID NO: 146 - protein, 114 aa, synthetic construct
  REGION 1..114: note = Description of Artificial Sequence: Synthetic polypeptide
QVQLAESGGGLVQPGGSLRLTCTASGFTFGSFGMSWVRQAPGEGLEWVSAISADSSDKRYADSVKGRFTISRDNAKKMLYLEMNSLKSEDTAVYYCVIGRGSPASQGTQVTVSS

SEQ ID NO: 147 - protein, 10 aa, synthetic construct
  REGION 1..10: note = Description of Artificial Sequence: Synthetic peptide
GFTFGSFGMS

SEQ ID NO: 148 - protein, 10 aa, synthetic construct
  REGION 1..10: note = Description of Artificial Sequence: Synthetic peptide
AISADSSDKR

SEQ ID NO: 149 - protein, 17 aa, synthetic construct
  REGION 1..17: note = Description of Artificial Sequence: Synthetic peptide
AISADSSDKRYADSVKG

SEQ ID NO: 150 - protein, 123 aa, synthetic construct
  REGION 1..123: note = Description of Artificial Sequence: Synthetic polypeptide
QVQLVESGGGLVQPGGSLRLSCAASGFTFSNYWMYWVRVAPGKGLERISRDISTGGGYSYYADSVKGRFTISRDNAKNTLYLQMNSLKPEDTALYYCAKDREAQVDTLDFDYRGQGTQVTVSS

SEQ ID NO: 151 - protein, 5 aa, synthetic construct
  REGION 1..5: note = Description of Artificial Sequence: Synthetic peptide
NYWMY

SEQ ID NO: 152 - protein, 10 aa, synthetic construct
  REGION 1..10: note = Description of Artificial Sequence: Synthetic peptide
GFTFSNYWMY

SEQ ID NO: 153 - protein, 11 aa, synthetic construct
  REGION 1..11: note = Description of Artificial Sequence: Synthetic peptide
RDISTGGGYSY

SEQ ID NO: 154 - protein, 18 aa, synthetic construct
  REGION 1..18: note = Description of Artificial Sequence: Synthetic peptide
RDISTGGGYSYYADSVKG

SEQ ID NO: 155 - protein, 13 aa, synthetic construct
  REGION 1..13: note = Description of Artificial Sequence: Synthetic peptide
DREAQVDTLDFDY

SEQ ID NO: 156 - protein, 117 aa, synthetic construct
  REGION 1..117: note = Description of Artificial Sequence: Synthetic polypeptide
AVQLVESGGGLVQGGGSLRLACAASERIFDLNLMGWYRQGPGNERELVATCITVGDSTNYADSVKGRFTISMDYTKQTVYLHMNSLRPEDTGLYYCKIRRTWHSELWGQGTQVTVSS

SEQ ID NO: 157 - protein, 5 aa, synthetic construct
  REGION 1..5: note = Description of Artificial Sequence: Synthetic peptide
LNLMG

SEQ ID NO: 158 - protein, 11 aa, synthetic construct
  REGION 1..11: note = Description of Artificial Sequence: Synthetic peptide
SERIFDLNLMG

SEQ ID NO: 159 - protein, 10 aa, synthetic construct
  REGION 1..10: note = Description of Artificial Sequence: Synthetic peptide
TCITVGDSTN

SEQ ID NO: 160 - protein, 17 aa, synthetic construct
  REGION 1..17: note = Description of Artificial Sequence: Synthetic peptide
TCITVGDSTNYADSVKG

SEQ ID NO: 161 - protein, 8 aa, synthetic construct
  REGION 1..8: note = Description of Artificial Sequence: Synthetic peptide
RRTWHSEL

SEQ ID NO: 162 - protein, 116 aa, synthetic construct
  REGION 1..116: note = Description of Artificial Sequence: Synthetic polypeptide
EVQLVESGGGLVQEGGSLRLACAASERIWDINLLGWYRQGPGNERELVATITVGDSTSYADSVKGRFTISRDYDKNTLYLQMNSLRPEDTGLYYCKIRRTWHSELWGQGTQVTVSS

SEQ ID NO: 163 - protein, 5 aa, synthetic construct
  REGION 1..5: note = Description of Artificial Sequence: Synthetic peptide
INLLG

SEQ ID NO: 164 - protein, 11 aa, synthetic construct
  REGION 1..11: note = Description of Artificial Sequence: Synthetic peptide
SERIWDINLLG

SEQ ID NO: 165 - protein, 9 aa, synthetic construct
  REGION 1..9: note = Description of Artificial Sequence: Synthetic peptide
TITVGDSTS

SEQ ID NO: 166 - protein, 16 aa, synthetic construct
  REGION 1..16: note = Description of Artificial Sequence: Synthetic peptide
TITVGDSTSYADSVKG

SEQ ID NO: 167 - protein, 116 aa, synthetic construct
  REGION 1..116: note = Description of Artificial Sequence: Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSSA

SEQ ID NO: 168 - protein, 116 aa, synthetic construct
  REGION 1..116: note = Description of Artificial Sequence: Synthetic polypeptide
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSSA

SEQ ID NO: 169 - protein, 116 aa, synthetic construct
  REGION 1..116: note = Description of Artificial Sequence: Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSSA

SEQ ID NO: 170 - protein, 115 aa, synthetic construct
  REGION 1..115: note = Description of Artificial Sequence: Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 171 - protein, 116 aa, synthetic construct
  REGION 1..116: note = Description of Artificial Sequence: Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 172 - protein, 117 aa, synthetic construct
  REGION 1..117: note = Description of Artificial Sequence: Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAA

SEQ ID NO: 173 - protein, 118 aa, synthetic construct
  REGION 1..118: note = Description of Artificial Sequence: Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAAA

SEQ ID NO: 174 - protein, 116 aa, synthetic construct
  REGION 1..116: note = Description of Artificial Sequence: Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSG

SEQ ID NO: 175 - protein, 117 aa, synthetic construct
  REGION 1..117: note = Description of Artificial Sequence: Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGG

SEQ ID NO: 176 - protein, 118 aa, synthetic construct
  REGION 1..118: note = Description of Artificial Sequence: Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGGG

SEQ ID NO: 177 - protein, 115 aa, synthetic construct
  REGION 1..115: note = Description of Artificial Sequence: Synthetic polypeptide
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 178 - protein, 10 aa, synthetic construct
  REGION 1..10: note = Description of Artificial Sequence: Synthetic peptide
GFTFSKFGMS

SEQ ID NO: 179 - protein, 16 aa, synthetic construct
  REGION 1..16: note = Description of Artificial Sequence: Synthetic peptide
SISGSGRDTLYAESVK

SEQ ID NO: 180 - protein, 6 aa, synthetic construct
  REGION 1..6: note = Description of Artificial Sequence: Synthetic peptide
GGSLSV

SEQ ID NO: 181 - protein, 115 aa, synthetic construct
  REGION 1..115: note = Description of Artificial Sequence: Synthetic polypeptide
EVQLVESGGGLVQPGNSLRLSCAASGFTFSRFGMSWVRQAPGKGLEWVSSISGSGSDTLYAESVKGRFTISRDNAKTTLYLQMNSLRPEDTAVYYCTIGGSLSRSSQGTLVTVSS

SEQ ID NO: 182 - protein, 10 aa, synthetic construct
  REGION 1..10: note = Description of Artificial Sequence: Synthetic peptide
GFTFSRFGMS

SEQ ID NO: 183 - protein, 16 aa, synthetic construct
  REGION 1..16: note = Description of Artificial Sequence: Synthetic peptide
SISGSGSDTLYAESVK

SEQ ID NO: 184 - protein, 115 aa, synthetic construct
  REGION 1..115: note = Description of Artificial Sequence: Synthetic polypeptide
EVQLVESGGGLVQPGNSLRLSCAASGFTFSKFGMSWVRQAPGKGLEWVSSISGSGTDTLYAESVKGRFTISRDNAKTTLYLQMNSLRPEDTAVYYCTIGGSLSRSSQGTLVTVSS

SEQ ID NO: 185 - protein, 16 aa, synthetic construct
  REGION 1..16: note = Description of Artificial Sequence: Synthetic peptide
SISGSGTDTLYAESVK

SEQ ID NO: 186 - protein, 119 aa, synthetic construct
  REGION 1..119: note = Description of Artificial Sequence: Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGGYFYDVWGQGTLVTVSS

SEQ ID NO: 187 - protein, 112 aa, synthetic construct
  REGION 1..112: note = Description of Artificial Sequence: Synthetic polypeptide
DIVMTQSPDSLAVSLGERATINCKSSQSLLNSRTGKNYLAWYQQKPGQPPKLLIYWASTRESGVPDRFSGSGSGTDFTLTISSLQAEDVAVYYCKQSYSRRTFGCGTKVEIK

SEQ ID NO: 188 - protein, 119 aa, synthetic construct
  REGION 1..119: note = Description of Artificial Sequence: Synthetic polypeptide
QVQLVQSGAEVKKPGASVKVSCKASGFNIKDYYMHWVRQAPGQRLEWMGWIDLENGNTIYDAKFQGRVTITRDTSASTAYMELSSLRSEDTAVYYCARDGYGRYFYDVWGQGTLVTVSS

SEQ ID NO: 189 - protein, 112 aa, synthetic construct
  REGION 1..112: note = Description of Artificial Sequence: Synthetic polypeptide
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYSRRTFGGGTKVEIK

SEQ ID NO: 190 - protein, 119 aa, synthetic construct
  REGION 1..119: note = Description of Artificial Sequence: Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDVYGRYLYDVWGQGTLVTVSS

SEQ ID NO: 191 - protein, 112 aa, synthetic construct
  REGION 1..112: note = Description of Artificial Sequence: Synthetic polypeptide
DIVMTQSPDSLAVSLGERATINCKSSQSLLNNRTRKNYLAWYQQKPGQPPKLLIYWASTRESGVPDRFSGSGSGTDFTLTISSLQAEDVAVYYCKQSYSRRTFGGGTKVEIK

SEQ ID NO: 192 - protein, 119 aa, synthetic construct
  REGION 1..119: note = Description of Artificial Sequence: Synthetic polypeptide
QVQLVQSGAEVKKPGASVKVSCKASGFNIKDYYMHWVRQAPGQRLEWMGWIDLENGNTIYDPKFQGRVTITRDTSASTAYMELSSLRSEDTAVYYCARDAYGRYFYDVWGQGTLVTVSS

SEQ ID NO: 193 - protein, 112 aa, synthetic construct
  REGION 1..112: note = Description of Artificial Sequence: Synthetic polypeptide
DIVMTQSPDSLAVSLGERATINCRSSQSLLNSRTRKNYLAWYQQKPGQPPKLLIYWASTRESGVPDRFSGSGSGTDFTLTISSLQAEDVAVYYCKQSYSRRTFGGGTKVEIK

SEQ ID NO: 194 - protein, 119 aa, synthetic construct
  REGION 1..119: note = Description of Artificial Sequence: Synthetic polypeptide
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSS

SEQ ID NO: 195 - protein, 112 aa, synthetic construct
  REGION 1..112: note = Description of Artificial Sequence: Synthetic polypeptide
DIVMTQSPDSLAVSLGERATINCKSSQSLLNGRTRKNYLAWYQQKPGQPPKLLIYWASTRESGVPDRFSGSGTGTDFTLTISSLQAEDVAVYYCKQSYSRRTFGGGTKVEIK

SEQ ID NO: 196 - protein, 119 aa, synthetic construct
  REGION 1..119: note = Description of Artificial Sequence: Synthetic polypeptide
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDNYGGYFYDVWGQGTLVTVSS

SEQ ID NO: 197 - protein, 112 aa, synthetic construct
  REGION 1..112: note = Description of Artificial Sequence: Synthetic polypeptide
DIVMTQSPDSLAVPLGERATINCKSSQSLLNSRTRKNYLAWYQQKPGQPPKLLIYWASTRESGVPDRFSGSGSGTDFTLTISSLQAEDVAVYYCKQSYSRRTFGGGTKVEIK

SEQ ID NO: 198 - protein, 119 aa, synthetic construct
  REGION 1..119: note = Description of Artificial Sequence: Synthetic polypeptide
QVQLVQSGAEVKKPGASVKVSCKASGFNIKDYYMHWVRQAPGQRLEWMGWIDLENGNTIYDPKFQGRVTITRDTSASTAYMELSSLRSEDTAVYYCARDGYGRYFYDVWGQGTLVTVSS

SEQ ID NO: 199 - protein, 112 aa, synthetic construct
  REGION 1..112: note = Description of Artificial Sequence: Synthetic polypeptide
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYSLRTFGGGTKVEIK

SEQ ID NO: 200 - protein, 119 aa, synthetic construct
  REGION 1..119: note = Description of Artificial Sequence: Synthetic polypeptide
QVQLVQSGAEVKKPGASVKVSCKASGFNIKDYYMHWVRQAPGQRLEWMGWIDLENGNTIYDPKFQGRVTITRDTSASTAYMELSSLRSEDTAVYYCARDGYGRYFFDVWGQGTLVTVSS

SEQ ID NO: 201 - protein, 112 aa, synthetic construct
  REGION 1..112: note = Description of Artificial Sequence: Synthetic polypeptide
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYNLRTFGGGTKVEIK

SEQ ID NO: 202 - protein, 119 aa, synthetic construct
  REGION 1..119: note = Description of Artificial Sequence: Synthetic polypeptide
QVQLVQSGAEVKKPGASVKVSCKASGFNIKDYYMHWVRQAPGQRLEWMGWIDLENGNTIYDPKFQGRVTITRDTSASTAYMELSSLRSEDTAVYYCAREGYGRYFYDVWGQGTLVTVSS

SEQ ID NO: 203 - protein, 112 aa, synthetic construct
  REGION 1..112: note = Description of Artificial Sequence: Synthetic polypeptide
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYFRRAFGGGTKVEIK

SEQ ID NO: 204 - protein, 119 aa, synthetic construct
  REGION 1..119: note = Description of Artificial Sequence: Synthetic polypeptide
QVQLVQSGAEVKKPGASVKVSCKASGFNIKDYYMHWVRQAPGQRLEWMGWIDLENGNTIYDPKFQGRVTITRDTSASTAYMELSSLRSEDTAVYYCARDGYGRYYYDVWGQGTLVTVSS

SEQ ID NO: 205 - protein, 112 aa, synthetic construct
  REGION 1..112: note = Description of Artificial Sequence: Synthetic polypeptide
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYNLRTFGGGTKLEIK

SEQ ID NO: 206 - protein, 119 aa, synthetic construct
  REGION 1..119: note = Description of Artificial Sequence: Synthetic polypeptide
QVQLVQSGAEVKKPGASVKVSCKASGFNIKDYYMHWVRQAPGQRLEWMGWIDLENGNTIYQPKFQGRVTITRDTSASTAYMELSSLRSEDTAVYYCARDGYGRYFYDVWGQGTLVTVSS

SEQ ID NO: 207 - protein, 112 aa, synthetic construct
  REGION 1..112: note = Description of Artificial Sequence: Synthetic polypeptide
DIVMTQSPDSLAVSLGERATINCKSSQSLLNSRTRKNYLAWYQQKPGQSPKLLIYWASTRESGVPDRFTGSGSGTDFTLTISSLQAEDVAVYYCKQSYSLRTFGGGTKVEIK

SEQ ID NO: 208 - protein, 119 aa, synthetic construct
  REGION 1..119: note = Description of Artificial Sequence: Synthetic polypeptide
QVQLVQSGAEVKKPGASVKVSCKASGFNIKDYYMHWVRQAPGQRLEWMGWIDLENGNTIYDPKFQGRVTITRDTSASTAYMELSSLRSEDTAVYYCARDGYGRYFYDYWGQGTLVTVSS

SEQ ID NO: 209 - protein, 112 aa, synthetic construct
  REGION 1..112: note = Description of Artificial Sequence: Synthetic polypeptide
DIVMTQSPDSLAVSLGERATINCKSSQSLLESRTGKNYLAWYQQKPGQPPKLLIYWASTRESGVPDRFSGSGSGTDFTLTISSLQAEDVAVYYCKQSYSLRTFGGGTKVEIK

SEQ ID NO: 210 - protein, 117 aa, synthetic construct
  REGION 1..117: note = Description of Artificial Sequence: Synthetic polypeptide
QVQLVESGGGLVQPGGSLRLSCAASGFTFSSYAMSWVRQAPGKGLEWVSGISASGGSTYYADSVKGRFTISRDNSKNTLYLQMNSLRAEDTAVYYCARYYLSQIDSWGQGTLVTVSS

SEQ ID NO: 211 - protein, 110 aa, synthetic construct
  REGION 1..110: note = Description of Artificial Sequence: Synthetic polypeptide
QSVLTQPPSVSAAPGQKVTISCSGSSSNIGNNYVSWYRQLPGTAPKLLIYENNKRPSGIPDRFSGSKSGTSATLGITGLQTGDEADYYCGTWDSSLRAGVFGTGTKVTVL

SEQ ID NO: 212 - protein, 249 aa, synthetic construct
  REGION 1..249: note = Description of Artificial Sequence: Synthetic polypeptide
QSVLTQPPSVSAAPGQKVTISCSGSSSNIGNNYVSWYRQLPGTAPKLLIYENNKRPSGIPDRFSGSKSGTSATLGITGLQTGDEADYYCGTWDSSLRAGVFGTGTKVTVLGSRGGGGSGGGGSGGGGSLEMAQVQLVESGGGLVQPGGSLRLSCAASGFTFSSYAMSWVRQAPGKGLEWVSGISASGGSTYYADSVKGRFTISRDNSKNTLYLQMNSLRAEDTAVYYCARYYLSQIDSWGQGTLVTVSS

SEQ ID NO: 213 - protein, 118 aa, synthetic construct
  REGION 1..118: note = Description of Artificial Sequence: Synthetic polypeptide
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATGIYSRPLGYWGQGTLVTVSS

SEQ ID NO: 214 - protein, 111 aa, synthetic construct
  REGION 1..111: note = Description of Artificial Sequence: Synthetic polypeptide
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGHVVFGGGTKLTVL

SEQ ID NO: 215 - protein, 251 aa, synthetic construct
  REGION 1..251: note = Description of Artificial Sequence: Synthetic polypeptide
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGHVVFGGGTKLTVLGSRGGGGSGGGGSGGGGSLEMAEVQLVQSGAEVKKPGATVKISCKVSGYTFTDYYMHWVQQAPGKGLEWMGLVDPEDGETIYAEKFQGRVTITADTSTDTAYMELSSLRSEDTAVYYCATGIYSRPLGYWGQGTLVTVSS

SEQ ID NO: 216 - protein, 121 aa, synthetic construct
  REGION 1..121: note = Description of Artificial Sequence: Synthetic polypeptide
EVQLVETGGGLVQPGGSLRLSCAASGFTFSSYAMSWVRQAPGKGLEWVSAISGSGGSTYYADSVKGRFTISRDNSKNTLYLQMNSLRAEDTAVYYCARSDGKHFWQQYDAWGQGTLVTVSS

SEQ ID NO: 217 - protein, 110 aa, synthetic construct
  REGION 1..110: note = Description of Artificial Sequence: Synthetic polypeptide
SYVLTQPPSASGTPGQRVTISCSGSSSNIGSHTVNWYQQLPGTAPKLLIYSNNQRPSGVPDRFSGSKSGTSASLAISGLQSEDEADYYCAAWDDSLNGYVFGTGTKVTVL

SEQ ID NO: 218 - protein, 253 aa, synthetic construct
  REGION 1..253: note = Description of Artificial Sequence: Synthetic polypeptide
SYVLTQPPSASGTPGQRVTISCSGSSSNIGSHTVNWYQQLPGTAPKLLIYSNNQRPSGVPDRFSGSKSGTSASLAISGLQSEDEADYYCAAWDDSLNGYVFGTGTKVTVLGSRGGGGSGGGGSGGGGSLEMAEVQLVETGGGLVQPGGSLRLSCAASGFTFSSYAMSWVRQAPGKGLEWVSAISGSGGSTYYADSVKGRFTISRDNSKNTLYLQMNSLRAEDTAVYYCARSDGKHFWQQYDAWGQGTLVTVSS

SEQ ID NO: 219 - protein, 114 aa, synthetic construct
  REGION 1..114: note = Description of Artificial Sequence: Synthetic polypeptide
EVQLVESGGGLVQPGGSLRLSCAASGFTVSSNYMSWVRQAPGKGLEWVSAISGSGGSTYYADSVKGRFTISRDNSKNTLYLQMNSLRAEDTAVYYCARMNIDYWGQGTLVTVSS

SEQ ID NO: 220 - protein, 107 aa, synthetic construct
  REGION 1..107: note = Description of Artificial Sequence: Synthetic polypeptide
DIQLTQSPSSLSAYVGDRVTITCRASQGITNSLAWYQQKPGKAPKLLLHAASRLESGVPSRFSGSGFGTDFTLTISSLQPEDFAVYYCQHYLGTPYSFGQGTKVEIK

SEQ ID NO: 221 - protein, 243 aa, synthetic construct
  REGION 1..243: note = Description of Artificial Sequence: Synthetic polypeptide
DIQLTQSPSSLSAYVGDRVTITCRASQGITNSLAWYQQKPGKAPKLLLHAASRLESGVPSRFSGSGFGTDFTLTISSLQPEDFAVYYCQHYLGTPYSFGQGTKVEIKRSRGGGGSGGGGSGGGGSLEMAEVQLVESGGGLVQPGGSLRLSCAASGFTVSSNYMSWVRQAPGKGLEWVSAISGSGGSTYYADSVKGRFTISRDNSKNTLYLQMNSLRAEDTAVYYCARMNIDYWGQGTLVTVSS

SEQ ID NO: 222 - protein, 124 aa, synthetic construct
  REGION 1..124: note = Description of Artificial Sequence: Synthetic polypeptide
EVQLVQSGAEVKRPGESLTISCKGSEYSFASYWITWVRQMPGKGLEWMGRIDPSDSYTNYSPSFQGHVTISADKSISTAYLQWSSLKASDTAIYYCARPFQYDYGGYSDAFDIWGQGTMVTVSS

SEQ ID NO: 223 - protein, 107 aa, synthetic construct
  REGION 1..107: note = Description of Artificial Sequence: Synthetic polypeptide
EIVLTQSPSSLSASVGDRVTISCRASQSVSRFLNWYQQKPGKAPKLLIYGVSTLERGVPSRFSGSGSGTDFTLTISSLQPEDFATYYCQESYIIPLTFGGGTKLEIK

SEQ ID NO: 224 - protein, 253 aa, synthetic construct
  REGION 1..253: note = Description of Artificial Sequence: Synthetic polypeptide
EIVLTQSPSSLSASVGDRVTISCRASQSVSRFLNWYQQKPGKAPKLLIYGVSTLERGVPSRFSGSGSGTDFTLTISSLQPEDFATYYCQESYIIPLTFGGGTKLEIKRSRGGGGSGGGGSGGGGSLEMAEVQLVQSGAEVKRPGESLTISCKGSEYSFASYWITWVRQMPGKGLEWMGRIDPSDSYTNYSPSFQGHVTISADKSISTAYLQWSSLKASDTAIYYCARPFQYDYGGYSDAFDIWGQGTMVTVSS

SEQ ID NO: 225 - protein, 118 aa, synthetic construct
  REGION 1..118: note = Description of Artificial Sequence: Synthetic polypeptide
QMQLVQSGAEVKKAGSSVKVSCETSGGTFSSSSVNWVRQAPGQGLEWMGGIIPIVGTPNYAQKFQDRVTITAVESTFTAYMELSGLRSEDTAVYYCARGGYRDYMDVWGRGTTVTVSS

SEQ ID NO: 226 - protein, 111 aa, synthetic construct
  REGION 1..111: note = Description of Artificial Sequence: Synthetic polypeptide
EIVMTQSPLSLSVTPGEPASISCRSSQSLLDSNGFNSLDWYLQKPGQSPQLLIHLGSDRASGVPDRFSGSGSGTDFTLKISRVEAEDVGIYYCMQSLQIPTFGQGTKVEIK

SEQ ID NO: 227 - protein, 251 aa, synthetic construct
  REGION 1..251: note = Description of Artificial Sequence: Synthetic polypeptide
EIVMTQSPLSLSVTPGEPASISCRSSQSLLDSNGFNSLDWYLQKPGQSPQLLIHLGSDRASGVPDRFSGSGSGTDFTLKISRVEAEDVGIYYCMQSLQIPTFGQGTKVEIKRSRGGGGSGGGGSGGGGSLEMAQMQLVQSGAEVKKAGSSVKVSCETSGGTFSSSSVNWVRQAPGQGLEWMGGIIPIVGTPNYAQKFQDRVTITAVESTFTAYMELSGLRSEDTAVYYCARGGYRDYMDVWGRGTTVTVSS

SEQ ID NO: 228 - protein, 117 aa, synthetic construct
  REGION 1..117: note = Description of Artificial Sequence: Synthetic polypeptide
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARGGFGAEFDYWGQGTLVTVSS

SEQ ID NO: 229 - protein, 110 aa, synthetic construct
  REGION 1..110: note = Description of Artificial Sequence: Synthetic polypeptide
SYELTQPPSASGTPGQRVTISCSGSSSNIGSNYVYWYQQLPGTAPKLLIYRNNQRPSGVPDRFSGSKSGTSASLAISGLRSEDEADYYCAAWDDSLSGYVFGTGTKVTVL

SEQ ID NO: 230 - protein, 249 aa, synthetic construct
  REGION 1..249: note = Description of Artificial Sequence: Synthetic polypeptide
SYELTQPPSASGTPGQRVTISCSGSSSNIGSNYVYWYQQLPGTAPKLLIYRNNQRPSGVPDRFSGSKSGTSASLAISGLRSEDEADYYCAAWDDSLSGYVFGTGTKVTVLGSRGGGGSGGGGSGGGGSLEMAEVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARGGFGAEFDYWGQGTLVTVSS

SEQ ID NO: 231 - protein, 123 aa, synthetic construct
  REGION 1..123: note = Description of Artificial Sequence: Synthetic polypeptide
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARGGISDDYYGSGSYDNWGQGTLVTVSS

SEQ ID NO: 232 - protein, 107 aa, synthetic construct
  REGION 1..107: note = Description of Artificial Sequence: Synthetic polypeptide
SYVLTQPPSVSVSPGQTASITCSGDKLGDKYASWYQQKPGQSPVLVIYQDNKRPSGIPERFSGSNSGNTATLTISGTQAMDEADYYCQAWDSSTEDVFGPGTKVTVL

SEQ ID NO: 233 - protein, 252 aa, synthetic construct
  REGION 1..252: note = Description of Artificial Sequence: Synthetic polypeptide
SYVLTQPPSVSVSPGQTASITCSGDKLGDKYASWYQQKPGQSPVLVIYQDNKRPSGIPERFSGSNSGNTATLTISGTQAMDEADYYCQAWDSSTEDVFGPGTKVTVLGSRGGGGSGGGGSGGGGSLEMAEVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARGGISDDYYGSGSYDNWGQGTLVTVSS

SEQ ID NO: 234 - protein, 118 aa, synthetic construct
  REGION 1..118: note = Description of Artificial Sequence: Synthetic polypeptide
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCARERGMGYAFDIWGQGTMVTVSS

SEQ ID NO: 235 - protein, 107 aa, synthetic construct
  REGION 1..107: note = Description of Artificial Sequence: Synthetic polypeptide
DIQLTQSPSSLSASVGDRVTITCRASQSISSYLNWYQQKPGKAPKLLIYAASSLQSGVPSRFSGSGSGTDFTLTISSLQPEDFATYYCQQSYSTPFTFGGGTKVEIK

SEQ ID NO: 236 - protein, 247 aa, synthetic construct
  REGION 1..247: note = Description of Artificial Sequence: Synthetic polypeptide
DIQLTQSPSSLSASVGDRVTITCRASQSISSYLNWYQQKPGKAPKLLIYAASSLQSGVPSRFSGSGSGTDFTLTISSLQPEDFATYYCQQSYSTPFTFGGGTKVEIKRSRGGGGSGGGGSGGGGSLEMAEVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCARERGMGYAFDIWGQGTMVTVSS

SEQ ID NO: 237 - protein, 114 aa, synthetic construct
  REGION 1..114: note = Description of Artificial Sequence: Synthetic polypeptide
QLQLQESGPGLVKPSETLSLTCSVSGVSMSENYWSWIRQPPGKRLEWIGCAHYTGDTHYNPSLKGRVTISLDTSMNQFSLRLNSVTAADTAVYYCASYHPFNYWGQGTLVTVSS

SEQ ID NO: 238 - protein, 104 aa, synthetic construct
  REGION 1..104: note = Description of Artificial Sequence: Synthetic polypeptide
DIQMTQSPSSLSASVGDRVTITCRASQGIGSYLAWYQQKPGKAPKLLIYPASTLQSGVPSRFSGSGSGTEFTLTISSLQPEDFATYYCQQLNSLFGQGTRLEIK

SEQ ID NO: 239 - protein, 240 aa, synthetic construct
  REGION 1..240: note = Description of Artificial Sequence: Synthetic polypeptide
DIQMTQSPSSLSASVGDRVTITCRASQGIGSYLAWYQQKPGKAPKLLIYPASTLQSGVPSRFSGSGSGTEFTLTISSLQPEDFATYYCQQLNSLFGQGTRLEIKRSRGGGGSGGGGSGGGGSLEMAQLQLQESGPGLVKPSETLSLTCSVSGVSMSENYWSWIRQPPGKRLEWIGCAHYTGDTHYNPSLKGRVTISLDTSMNQFSLRLNSVTAADTAVYYCASYHPFNYWGQGTLVTVSS

SEQ ID NO: 240 - protein, 126 aa, synthetic construct
  REGION 1..126: note = Description of Artificial Sequence: Synthetic polypeptide
EVQLVQSGAEVRRPGATVKISCKVSGYTFNDFYLHWVRQAPGKGLEWMGRIDPEDGKTRYAEKFQGRLTITADTSTDTLYMQLGGLTSDDTAVYYCTTDWGYSSSLREEDIWYDCWGQGTLVTVSS

SEQ ID NO: 241 - protein, 110 aa, synthetic construct
  REGION 1..110: note = Description of Artificial Sequence: Synthetic polypeptide
QAVLTQPPSASGTPGQRVTISCSGSSSNIGTKTVNWYQVLPGTAPKLLIYSNYRRPSGVPDRFSGSKSGTSASLAISGLQSDDEADYYCALWDDSLDGYVFGTGTKVTVL

SEQ ID NO: 242 - protein, 258 aa, synthetic construct
  REGION 1..258: note = Description of Artificial Sequence: Synthetic polypeptide
QAVLTQPPSASGTPGQRVTISCSGSSSNIGTKTVNWYQVLPGTAPKLLIYSNYRRPSGVPDRFSGSKSGTSASLAISGLQSDDEADYYCALWDDSLDGYVFGTGTKVTVLGSRGGGGSGGGGSGGGGSLEMAEVQLVQSGAEVRRPGATVKISCKVSGYTFNDFYLHWVRQAPGKGLEWMGRIDPEDGKTRYAEKFQGRLTITADTSTDTLYMQLGGLTSDDTAVYYCTTDWGYSSSLREEDIWYDCWGQGTLVTVSS

SEQ ID NO: 243 - protein, 122 aa, synthetic construct
  REGION 1..122: note = Description of Artificial Sequence: Synthetic polypeptide
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDYGYGDYGDAFDIWGQGTMVTVSS

SEQ ID NO: 244 - protein, 108 aa, synthetic construct
  REGION 1..108: note = Description of Artificial Sequence: Synthetic polypeptide
SYELTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVL

SEQ ID NO: 245 - protein, 252 aa, synthetic construct
  REGION 1..252: note = Description of Artificial Sequence: Synthetic polypeptide
SYELTQPPSVSVAPGKTARITCGGNNIGSKSVHWYQQKPGQAPVLVIYYDSDRPSGIPERFSGSNSGNTATLTISRVEAGDEADYYCQVWDSSSDHYVFGTGTKVTVLGSRGGGGSGGGGSGGGGSLEMAEVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDYGYGDYGDAFDIWGQGTMVTVSS

SEQ ID NO: 246 - protein, 121 aa, synthetic construct
  REGION 1..121: note = Description of Artificial Sequence: Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARVVGTIYSMQYDVWGQGTLVTVSS

SEQ ID NO: 247 - protein, 108 aa, synthetic construct
  REGION 1..108: note = Description of Artificial Sequence: Synthetic polypeptide
SYVLTQPPSVSVAPGKTARITCGGNNIGSKSVHWYQQRPGQAPVLVVYDDSDRPSGIPERFSGSNSGNTATLTISRVEAGDEADYSCQVWDSSSDHYVFGPGTKVTVL

SEQ ID NO: 248 - protein, 251 aa, synthetic construct
  REGION 1..251: note = Description of Artificial Sequence: Synthetic polypeptide
SYVLTQPPSVSVAPGKTARITCGGNNIGSKSVHWYQQRPGQAPVLVVYDDSDRPSGIPERFSGSNSGNTATLTISRVEAGDEADYSCQVWDSSSDHYVFGPGTKVTVLGSRGGGGSGGGGSGGGGSLEMAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARVVGTIYSMQYDVWGQGTLVTVSS

SEQ ID NO: 249 - protein, 130 aa, synthetic construct
  REGION 1..130: note = Description of Artificial Sequence: Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQVWGWQGGMYPRSNWWYNMDSWGQGTLVTVSS

SEQ ID NO: 250 - protein, 108 aa, synthetic construct
  REGION 1..108: note = Description of Artificial Sequence: Synthetic polypeptide
LPVLTQPPSVSVAPGKTARITCGGNNIGSKSVHWYQQKPGQAPVLVVYDDSDRPSGIPERFSGSNSGNTATLTISRVEAGDEADYYCQVWDSSSDYVVFGGGTKLTVL

SEQ ID NO: 251 - protein, 260 aa, synthetic construct
  REGION 1..260: note = Description of Artificial Sequence: Synthetic polypeptide
LPVLTQPPSVSVAPGKTARITCGGNNIGSKSVHWYQQKPGQAPVLVVYDDSDRPSGIPERFSGSNSGNTATLTISRVEAGDEADYYCQVWDSSSDYVVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQVWGWQGGMYPRSNWWYNMDSWGQGTLVTVSS

SEQ ID NO: 252 - protein, 121 aa, synthetic construct
  REGION 1..121: note = Description of Artificial Sequence: Synthetic polypeptide
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARWSSTWDSMYMDYWGQGTLVTVSS

SEQ ID NO: 253 - protein, 110 aa, synthetic construct
  REGION 1..110: note = Description of Artificial Sequence: Synthetic polypeptide
QAVLTQPPSVSEAPRQRVTISCSGSSSNVGNNAVNWYQQVPGKAPKLLIYYDDLLSSGVSDRFSGSKSGTSASLAISGLQSEDEADYYCAAWDDSLNGPVFGGGTKLTVL

SEQ ID NO: 254 - protein, 253 aa, synthetic construct
  REGION 1..253: note = Description of Artificial Sequence: Synthetic polypeptide
QAVLTQPPSVSEAPRQRVTISCSGSSSNVGNNAVNWYQQVPGKAPKLLIYYDDLLSSGVSDRFSGSKSGTSASLAISGLQSEDEADYYCAAWDDSLNGPVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARWSSTWDSMYMDYWGQGTLVTVSS

SEQ ID NO: 255 - protein, 121 aa, synthetic construct
  REGION 1..121: note = Description of Artificial Sequence: Synthetic polypeptide
EVQLVQSGAEVKKPGESLRISCKGSGYSFTSYWIGWVRQMPGKGLEWMGIIYPGDSDTRYSPSFQGQVTISADKSISTAYLQWSSLKASDTAMYYCARVTYSMDSYYFDSWGQGTLVTVSS

SEQ ID NO: 256 - protein, 108 aa, synthetic construct
  REGION 1..108: note = Description of Artificial Sequence: Synthetic polypeptide
QPVLTQPPSVSVAPGKTARITCGGNNIGSESVHWYQQKPGQAPMVVIYYDSNRPSGIPERFSGSNSGNTATLTVSRVEAEDEADYYCQVWNSSSDHRGVFGGGTKLTV

SEQ ID NO: 257 - protein, 252 aa, synthetic construct
  REGION 1..252: note = Description of Artificial Sequence: Synthetic polypeptide
QPVLTQPPSVSVAPGKTARITCGGNNIGSESVHWYQQKPGQAPMVVIYYDSNRPSGIPERFSGSNSGNTATLTVSRVEAEDEADYYCQVWNSSSDHRGVFGGGTKLTVLGSRGGGGSGGGGSGGGGSLEMAEVQLVQSGAEVKKPGESLRISCKGSGYSFTSYWIGWVRQMPGKGLEWMGIIYPGDSDTRYSPSFQGQVTISADKSISTAYLQWSSLKASDTAMYYCARVTYSMDSYYFDSWGQGTLVTVSS

SEQ ID NO: 258 - protein, 243 aa, synthetic construct
  REGION 1..243: note = Description of Artificial Sequence: Synthetic polypeptide
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 259 - protein, 118 aa, synthetic construct [sequence omitted]

SEQ ID NO: 260 - protein, 108 aa, synthetic construct [sequence omitted]

SEQ ID NO: 261 - protein, 5 aa, synthetic construct
  REGION 1..5: note = Description of Artificial Sequence: Synthetic peptide
GYGMH

SEQ ID NO: 262 - protein, 17 aa, synthetic construct
  REGION 1..17: note = Description of Artificial Sequence: Synthetic peptide
VIWYDGSKKYYVDSVKG

SEQ ID NO: 263 - protein, 9 aa, synthetic construct
  REGION 1..9: note = Description of Artificial Sequence: Synthetic peptide
QMGYWHFDL

SEQ ID NO: 264 - protein, 11 aa, synthetic construct
  REGION 1..11: note = Description of Artificial Sequence: Synthetic peptide
RASQSVSSYLA

SEQ ID NO: 265 - protein, 7 aa, synthetic construct
  REGION 1..7: note = Description of Artificial Sequence: Synthetic peptide
DASNRAT

SEQ ID NO: 266 - protein, 10 aa, synthetic construct
  REGION 1..10: note = Description of Artificial Sequence: Synthetic peptide
QQRSNWPPLT

SEQ ID NO: 267 - protein, 119 aa, synthetic construct [sequence omitted]

SEQ ID NO: 268 - protein, 101 aa, synthetic construct
  REGION 1..101: note = Description of Artificial Sequence: Synthetic polypeptide
DFMLTQPHSVSESPGKTVIISCWYQQRPGRAPTTVIFGVPDRFSGSIDRSSNSASLTISGLQTEDEADYYCFGGGTKLTVLGQPKAAPSVTLFPPSSEELQ

SEQ ID NO: 269 - protein, 119 aa, synthetic construct [sequence omitted]

SEQ ID NO: 270 - protein, 111 aa, synthetic construct
  REGION 1..111: note = Description of Artificial Sequence: Synthetic polypeptide
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLR

SEQ ID NO: 271 - protein, 119 aa, synthetic construct
  REGION 1..119: note = Description of Artificial Sequence: Synthetic polypeptide
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 272 - protein, 106 aa, synthetic construct
  REGION 1..106: note = Description of Artificial Sequence: Synthetic polypeptide
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 273 - protein, 243 aa, synthetic construct
  REGION 1..243: note = Description of Artificial Sequence: Synthetic polypeptide
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 274 - protein, 119 aa, synthetic construct [sequence omitted]

SEQ ID NO: 275 - protein, 107 aa, synthetic construct
  REGION 1..107: note = Description of Artificial Sequence: Synthetic polypeptide
DDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQIT

SEQ ID NO: 276 - protein, 119 aa, synthetic construct
  REGION 1..119: note = Description of Artificial Sequence: Synthetic polypeptide
QVQLVQSGGGVVQPGRSLRLSCKASGYTFTRYTMHWVRQAPGKGLEWIGYINPSRGYTNYNQKFKDRFTISTDKSKSTAFLQMDSLRPEDTAVYYCARYYDDHYCLDYWGQGTPVTVSS

SEQ ID NO: 277 - protein, 107 aa, synthetic construct
  REGION 1..107: note = Description of Artificial Sequence: Synthetic polypeptide
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 278 - protein, 125 aa, synthetic construct
  REGION 1..125: note = Description of Artificial Sequence: Synthetic polypeptide
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 279 - protein, 109 aa, synthetic construct
  REGION 1..109: note = Description of Artificial Sequence: Synthetic polypeptide
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 280 - protein, 123 aa, synthetic construct
  REGION 1..123: note = Description of Artificial Sequence: Synthetic polypeptide
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVAS

SEQ ID NO: 281 - protein, 108 aa, synthetic construct
  REGION 1..108: note = Description of Artificial Sequence: Synthetic polypeptide
AEIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIK

SEQ ID NO: 282 - protein, 45 aa, synthetic construct
  REGION 1..45: note = Description of Artificial Sequence: Synthetic polypeptide
LKEAKEKAIEELKKAGITSDYYFDLINKAKTVEGVNALKDEILKA

SEQ ID NO: 283 - protein, 123 aa, synthetic construct
  REGION 1..123: note = Description of Artificial Sequence: Synthetic polypeptide
EVQLLESGGGLVQPGGSLRLSCAASGFTFSKYWMSWVRQAPGKGLEWVSSIDFMGPHTYYADSVKGRFTISRDNSKNTLYLQMNSLRAEDTAVYYCAKGRTSMLPMKGKFDYWGQGTLVTVSS

SEQ ID NO: 284 - protein, 118 aa, synthetic construct
  REGION 1..118: note = Description of Artificial Sequence: Synthetic polypeptide
EVQLLESGGGLVQPGGSLRLSCTASGFTFDEYNMSWVRQAPGKGLEWVSTILPHGDRTYYADSVKGRFTISRDNSKNTLYLQMNSLRAEDTAVYYCAKQDPLYRFDYWGQGTLVTVSS

SEQ ID NO: 285 - protein, 108 aa, synthetic construct
  REGION 1..108: note = Description of Artificial Sequence: Synthetic polypeptide
DIQMTQSPSSLSASVGDRVTITCRASQKIATYLNWYQQKPGKAPKLLIYRSSSLQSAVPSRFSGSGSGTVFTLTISSLQPEDFATYYCQQTYAVPPTFGQGTKVEIKR

SEQ ID NO: 286 - protein, 108 aa, synthetic construct
  REGION 1..108: note = Description of Artificial Sequence: Synthetic polypeptide
DIQMTQSPSSLSASVGDRVTITCRASQSISSYLNWYQQKPGKAPKLLIYRNSPLQSGVPSRFSGSGSGTDFTLTISSLQPEDFATYYCQQTYRVPPTFGQGTKVEIKR

SEQ ID NO: 287 - protein, 115 aa, synthetic construct
  REGION 1..115: note = Description of Artificial Sequence: Synthetic polypeptide
QVQLQESGGGLVQPGGSLRLSCEASGFTFSRFGMTWVRQAPGKGVEWVSGISSLGDSTLYADSVKGRFTISRDNAKNTLYLQMNSLKPEDTAVYYCTIGGSLNPGGQGTQVTVSS

SEQ ID NO: 288 - protein, 121 aa, synthetic construct
  REGION 1..121: note = Description of Artificial Sequence: Synthetic polypeptide
EVQLLESGGGLVQPGGSLRLSCAVSGIDLSNYAINWVRQAPGKCLEWIGIIWASGTTFYATWAKGRFTISRDNSKNTVYLQMNSLRAEDTAVYYCARTVPGYSTAPYFDLWGQGTLVTVSS

SEQ ID NO: 289 - protein, 112 aa, synthetic construct
  REGION 1..112: note = Description of Artificial Sequence: Synthetic polypeptide
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGCGTKVEIKRT

SEQ ID NO: 290 - protein, 121 aa, synthetic construct
  REGION 1..121: note = Description of Artificial Sequence: Synthetic polypeptide
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSS

SEQ ID NO: 291 - protein, 112 aa, synthetic construct
  REGION 1..112: note = Description of Artificial Sequence: Synthetic polypeptide
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIKRT

SEQ ID NO: 292 - protein, 20 aa, synthetic construct
  REGION 1..20: note = Description of Artificial Sequence: Synthetic peptide
GSGSGSGSGSGSGSGSGSGS

SEQ ID NO: 293 - protein, 30 aa, synthetic construct
  REGION 1..30: note = Description of Artificial Sequence: Synthetic polypeptide
GGSGGSGGSGGSGGSGGSGGSGGSGGSGGS

SEQ ID NO: 294 - protein, 40 aa, synthetic construct
  REGION 1..40: note = Description of Artificial Sequence: Synthetic polypeptide
GGGSGGGSGGGSGGGSGGGSGGGSGGGSGGGSGGGSGGGS

SEQ ID NO: 295 - protein, 40 aa, synthetic construct
  REGION 1..40: note = Description of Artificial Sequence: Synthetic polypeptide
GGSGGGSGGGSGGGSGGGSGGGSGGGSGGGSGGGSGGGSG

SEQ ID NO: 296 - protein, 50 aa, synthetic construct
  REGION 1..50: note = Description of Artificial Sequence: Synthetic polypeptide
GGSGGGGSGGGGSGGGGSGGGGSGGGGSGGGGSGGGGSGGGGSGGGGSGG

SEQ ID NO: 297 - protein, 50 aa, synthetic construct
  REGION 1..50: note = Description of Artificial Sequence: Synthetic polypeptide
GGGGSGGGGSGGGGSGGGGSGGGGSGGGGSGGGGSGGGGSGGGGSGGGGS

SEQ ID NO: 298 - protein, 20 aa, synthetic construct
  REGION 1..20: note = Description of Artificial Sequence: Synthetic peptide
GGGGSGGGGSGGGGSGGGGS

SEQ ID NO: 299 - protein, 15 aa, synthetic construct
  REGION 1..15: note = Description of Artificial Sequence: Synthetic peptide
GGGGSGGGGSGGGGS

SEQ ID NO: 300 - protein, 100 aa, synthetic construct
  REGION 1..100: note = Description of Artificial Sequence: Synthetic polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 301 - protein, 100 aa, synthetic construct
  REGION 1..100: note = Description of Artificial Sequence: Synthetic polypeptide
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 302 - protein, 9 aa, synthetic construct
  REGION 1..9: note = Description of Artificial Sequence: Synthetic peptide
GGGGSGGGS

SEQ ID NO: 303 - protein, 642 aa, synthetic construct
  REGION 1..642: note = Description of Artificial Sequence: Synthetic polypeptide
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSGGGGSGGGGSGGGGSDIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKGGGGSGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHHHHHH

SEQ ID NO: 304 - protein, 642 aa, synthetic construct
  REGION 1..642: note = Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSQVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSGGGGSGGGGSGGGGSDIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHHHHHH

SEQ ID NO: 305 - protein, 642 aa, synthetic construct
  REGION 1..642: note = Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSGGGGSGGGGSGGGGSDIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKHHHHHHHHHH

SEQ ID NO: 306 - protein, 642 aa, synthetic construct
  REGION 1..642: note = Description of Artificial Sequence: Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSQVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSGGGGSGGGGSGGGGSDIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKHHHHHHHHHH

SEQ ID NO: 307 - protein, 642 aa, synthetic construct
  REGION 1..642: note = Description of Artificial Sequence: Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSGGGGSGGGGSGGGGSDIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKGGGGSGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSHHHHHHHHHH

SEQ ID NO: 308 - protein, 636 aa, synthetic construct
  REGION 1..636: note = Description of Artificial Sequence: Synthetic polypeptide
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRGGGGSGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHHHHHH

SEQ ID NO: 309 - protein, 636 aa, synthetic construct
  REGION 1..636: note = Description of Artificial Sequence: Synthetic polypeptide
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSHHHHHHHHHH

SEQ ID NO: 310 - protein, 636 aa, synthetic construct
  REGION 1..636: note = Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSQVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSHHHHHHHHHH

SEQ ID NO: 311 - protein, 636 aa, synthetic construct
  REGION 1..636: note = Description of Artificial Sequence: Synthetic polypeptide
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRHHHHHHHHHH

SEQ ID NO: 312 - protein, 636 aa, synthetic construct
  REGION 1..636: note = Description of Artificial Sequence: Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGSQVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRHHHHHHHHHH

SEQ ID NO: 313 - protein, 636 aa, synthetic construct
  REGION 1..636: note = Description of Artificial Sequence: Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRGGGGSGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSHHHHHHHHHH

SEQ ID NO: 314 - protein, 124 aa, synthetic construct; identical to SEQ ID NO: 81
  REGION 1..124: note = Description of Artificial Sequence: Synthetic polypeptide

SEQ ID NO: 315 - protein, 107 aa, synthetic construct; identical to SEQ ID NO: 82
  REGION 1..107: note = Description of Artificial Sequence: Synthetic polypeptide

SEQ ID NO: 316 - protein, 121 aa, synthetic construct
  REGION 1..121: note = Description of Artificial Sequence: Synthetic polypeptide
EVQLQQSGPELVKPGASVKMSCKASGYTFTNYVIHWVKQKPGQGLEWIGYFNPYNDGTEYNEKFKAKATLTSDKSSSTAYMELSSLTSEDSAVYYCAKGPYYYGSSPFDYWGQGTTLTVSS

SEQ ID NO: 317 - protein, 112 aa, synthetic construct
  REGION 1..112: note = Description of Artificial Sequence: Synthetic polypeptide
DAVMTQTPLSLPVSLGDQASISCRSSQSLENSNGNTYLNWYLQKPGQSPQLLIYRVSNRFSGVLDRFSGSGSGTDFTLKISRVEAEDLGVYFCLQVTHVPYTFGGGTKLEIK

SEQ ID NO: 318 - protein, 10 aa, synthetic construct
  REGION 1..10: note = Description of Artificial Sequence: Synthetic peptide
GYTFTNYVIH

SEQ ID NO: 319 - protein, 17 aa, synthetic construct
  REGION 1..17: note = Description of Artificial Sequence: Synthetic peptide
YFNPYNDGTEYNEKFKA

SEQ ID NO: 320 - protein, 12 aa, synthetic construct
  REGION 1..12: note = Description of Artificial Sequence: Synthetic peptide
GPYYYGSSPFDY

SEQ ID NO: 321 - protein, 16 aa, synthetic construct
  REGION 1..16: note = Description of Artificial Sequence: Synthetic peptide
RSSQSLENSNGNTYLN

SEQ ID NO: 322 - protein, 7 aa, synthetic construct
  REGION 1..7: note = Description of Artificial Sequence: Synthetic peptide
RVSNRFS

SEQ ID NO: 323 - protein, 7 aa, synthetic construct
  REGION 1..7: note = Description of Artificial Sequence: Synthetic peptide
RVSNRFS

SEQ ID NO: 324 - protein, 121 aa, synthetic construct
  REGION 1..121: note = Description of Artificial Sequence: Synthetic polypeptide
QVQLQQSGAELVRPGSSVKISCKASGYAFSTYWMNWVKQRPGQGLEWIGQIYPGDDDTKYNGKFKGKASLTADKSSSTAYMQLISLTSEDSAVYFCARRYFRYDYWYSDVWGAGTTVTVTS

SEQ ID NO: 325 - protein, 107 aa, synthetic construct
  REGION 1..107: note = Description of Artificial Sequence: Synthetic polypeptide
DIQMTQTTSSLSASLGDRVTISCRASQDISNYLNWYQQKPDGTVKLLIYYTSRLHSGVPARFSGSGSGTDYSLTISNLEQEDIATYFCHQGNTLPLTFGAGTKLELK

SEQ ID NO: 326 - protein, 10 aa, synthetic construct
  REGION 1..10: note = Description of Artificial Sequence: Synthetic peptide
GYAFSTYWMN

SEQ ID NO: 327 - protein, 17 aa, synthetic construct
  REGION 1..17: note = Description of Artificial Sequence: Synthetic peptide
QIYPGDDDTKYNGKFKG

SEQ ID NO: 328 - protein, 12 aa, synthetic construct
  REGION 1..12: note = Description of Artificial Sequence: Synthetic peptide
RYFRYDYWYSDV

SEQ ID NO: 329 - protein, 11 aa, synthetic construct
  REGION 1..11: note = Description of Artificial Sequence: Synthetic peptide
RASQDISNYLN

SEQ ID NO: 330 - protein, 7 aa, synthetic construct
  REGION 1..7: note = Description of Artificial Sequence: Synthetic peptide
YTSRLHS

SEQ ID NO: 331 - protein, 9 aa, synthetic construct
  REGION 1..9: note = Description of Artificial Sequence: Synthetic peptide
HQGNTLPLT

SEQ ID NO: 332 - protein, 116 aa, synthetic construct
  REGION 1..116: note = Description of Artificial Sequence: Synthetic polypeptide
EIQLQQSGPELVKPGASVKVSCKASGYAFTSYNMYWVKQSHGKSLEWIGYIDPYNGDTTYNQKFKGKATLTVDKSSSTAYMHLNSLTSEDSAVYYCLTTAYAMDYWGQGTSVTVSS

SEQ ID NO: 333 - protein, 106 aa, synthetic construct
  REGION 1..106: note = Description of Artificial Sequence: Synthetic polypeptide
QIVLTQSPAIMSASLGEEITLTCSASSTVNYMHWYQQKSGTSPKLLIYSTSNLASGVPSRFSGSGSGTFYSLTIRSVEAEDAADYYCHQWSSYPYTFGGGTKLEIK

SEQ ID NO: 334 - protein, 10 aa, synthetic construct
  REGION 1..10: note = Description of Artificial Sequence: Synthetic peptide
GYAFTSYNMY

SEQ ID NO: 335 - protein, 17 aa, synthetic construct
  REGION 1..17: note = Description of Artificial Sequence: Synthetic peptide
YIDPYNGDTTYNQKFKG

SEQ ID NO: 336 - protein, 7 aa, synthetic construct
  REGION 1..7: note = Description of Artificial Sequence: Synthetic peptide
TAYAMDY

SEQ ID NO: 337 - protein, 10 aa, synthetic construct
  REGION 1..10: note = Description of Artificial Sequence: Synthetic peptide
SASSTVNYMH

SEQ ID NO: 338 - protein, 7 aa, synthetic construct
  REGION 1..7: note = Description of Artificial Sequence: Synthetic peptide
STSNLAS

SEQ ID NO: 339 - protein, 9 aa, synthetic construct
  REGION 1..9: note = Description of Artificial Sequence: Synthetic peptide
HQWSSYPYT

SEQ ID NO: 340 - protein, 121 aa, synthetic construct
  REGION 1..121: note = Description of Artificial Sequence: Synthetic polypeptide
QVQLVQSGAEVKKPGSSVKVSCKASGYTFTDYVIHWVRQAPGQGLEWMGYFNPYNDGTEYNEKFKARVTITADKSTSTAYMELSSLRSEDTAVYYCARGPYYYGSSPFDYWGQGTTVTVSS

SEQ ID NO: 341 - protein, 10 aa, synthetic construct
  REGION 1..10: note = Description of Artificial Sequence: Synthetic peptide
GYTFTNYVIH

SEQ ID NO: 342 - protein, 17 aa, synthetic construct
  REGION 1..17: note = Description of Artificial Sequence: Synthetic peptide
YFNPYNDGTEYNEKFKA

SEQ ID NO: 343 - protein, 12 aa, synthetic construct
  REGION 1..12: note = Description of Artificial Sequence: Synthetic peptide
GPYYYGSSPFDY

SEQ ID NO: 344 - protein, 112 aa, synthetic construct
  REGION 1..112: note = Description of Artificial Sequence: Synthetic polypeptide
DIVMTQTPLSLPVTPGEPASISCRSSQSLENSNHNTYINWYLQKPGQSPQLLIYRVSKRFSGVPDRFSGSGSGTDFTLKISRVEAEDVGVYYCHQVTHVPYTFGQGTKLEIK

SEQ ID NO: 345 - protein, 16 aa, synthetic construct
  REGION 1..16: note = Description of Artificial Sequence: Synthetic peptide
RSSQSLENSNHNTYIN

SEQ ID NO: 346 - protein, 7 aa, synthetic construct
  REGION 1..7: note = Description of Artificial Sequence: Synthetic peptide
RVSKRFS

SEQ ID NO: 347 - protein, 9 aa, synthetic construct
  REGION 1..9: note = Description of Artificial Sequence: Synthetic peptide
HQVTHVPYT

SEQ ID NO: 348 - protein, 121 aa, synthetic construct
  REGION 1..121: note = Description of Artificial Sequence: Synthetic polypeptide
QVQLVQSGAEVKKPGASVKVSCKASGYAFSTYWMNWVRQAPGQGLEWMGQIYPGDDDTKYSGKFKGRVTITADKSTSTAYMELSSLRSEDTAVYYCARRYFRYDYWYSDVWGQGTTVTVSS

SEQ ID NO: 349 - protein, 107 aa, synthetic construct
  REGION 1..107: note = Description of Artificial Sequence: Synthetic polypeptide
DIQMTQSPSSLSASVGDRVTITCRASQDISNYLNWYQQKPGKVPKLLIYYTSRLHSGVPSRFSGSGSGTDFTLTISSLQPEDVATYYCHQGNTLPLTFGQGTKLEIK

SEQ ID NO: 350 - protein, 10 aa, synthetic construct
  REGION 1..10: note = Description of Artificial Sequence: Synthetic peptide
GYAFSTYWMN

SEQ ID NO: 351 - protein, 17 aa, synthetic construct
  REGION 1..17: note = Description of Artificial Sequence: Synthetic peptide
QIYPGDDDTKYSGKFKG

SEQ ID NO: 352 - protein, 12 aa, synthetic construct
  REGION 1..12: note = Description of Artificial Sequence: Synthetic peptide
RYFRYDYWYSDV

SEQ ID NO: 353 - protein, 11 aa, synthetic construct
  REGION 1..11: note = Description of Artificial Sequence: Synthetic peptide
RASQDISNYLN

SEQ ID NO: 354 - protein, 7 aa, synthetic construct
  REGION 1..7: note = Description of Artificial Sequence: Synthetic peptide
YTSRLHS

SEQ ID NO: 355 - protein, 9 aa, synthetic construct
  REGION 1..9: note = Description of Artificial Sequence: Synthetic peptide
HQGNTLPLT

SEQ ID NO: 356 - protein, 116 aa, synthetic construct
  REGION 1..116: note = Description of Artificial Sequence: Synthetic polypeptide
QMQLVQSGPEVKKPGTSVKVSCKASGYAFTSYNMYWVRQARGQRLEWIGYIDPYNADTTYNQKFKGRVTITRDMSTSTAYMELSSLRSEDTAVYYCLTTAYAMDYWGQGTLVTVSS

SEQ ID NO: 357 - protein, 106 aa, synthetic construct
  REGION 1..106: note = Description of Artificial Sequence: Synthetic polypeptide
DIQLTQSPSFLSASVGDRVTITCSASSTVNYMHWYQQKPGKAPKLLIYSTSNLASGVPSRFSGSGSGTEFTLTISSLQPEDFATYYCHQWSSYPYTFGQGTKLEIK

SEQ ID NO: 358 - protein, 10 aa, synthetic construct
  REGION 1..10: note = Description of Artificial Sequence: Synthetic peptide
GYAFTSYNMY

SEQ ID NO: 359 - protein, 17 aa, synthetic construct
  REGION 1..17: note = Description of Artificial Sequence: Synthetic peptide
YIDPYNADTTYNQKFKG

SEQ ID NO: 360 - protein, 7 aa, synthetic construct
  REGION 1..7: note = Description of Artificial Sequence: Synthetic peptide
TAYAMDY

SEQ ID NO: 361 - protein, 10 aa, synthetic construct
  REGION 1..10: note = Description of Artificial Sequence: Synthetic peptide
SASSTVNYMH

SEQ ID NO: 362 - protein, 7 aa, synthetic construct
  REGION 1..7: note = Description of Artificial Sequence: Synthetic peptide
STSNLAS

SEQ ID NO: 363 - protein, 9 aa, synthetic construct
  REGION 1..9: note = Description of Artificial Sequence: Synthetic peptide
HQWSSYPYT

SEQ ID NO: 364 - protein, 117 aa, synthetic construct
  REGION 1..117: note = Description of Artificial Sequence: Synthetic polypeptide
QVKLEESGGELVQPGGPLRLSCAASGNIFSINRMGWYRQAPGKQRAFVASITVRGITNYADSVKGRFTISVDKSKNTIYLQMNALKPEDTAVYYCNAVSSNRDPDYWGQGTQVTVSS

SEQ ID NO: 365 - protein, 5 aa, synthetic construct
  REGION 1..5: note = Description of Artificial Sequence: Synthetic peptide
INRMG

SEQ ID NO: 366 - protein, 16 aa, synthetic construct
  REGION 1..16: note = Description of Artificial Sequence: Synthetic peptide
SITVRGITNYADSVKG

SEQ ID NO: 367 - protein, 9 aa, synthetic construct
  REGION 1..9: note = Description of Artificial Sequence: Synthetic peptide
VSSNRDPDY

SEQ ID NO: 368 - protein, 12 aa, synthetic construct
  REGION 1..12: note = Description of Artificial Sequence: Synthetic peptide
ARDSYGRYFYDV

SEQ ID NO: 369 - protein, 12 aa, synthetic construct
  REGION 1..12: note = Description of Artificial Sequence: Synthetic peptide
ARDVYGRYFYDL

SEQ ID NO: 370 - protein, 12 aa, synthetic construct
  REGION 1..12: note = Description of Artificial Sequence: Synthetic peptide
ARDAYGGYFYDV

SEQ ID NO: 371 - protein, 125 aa, synthetic construct
  REGION 1..125: note = Description of Artificial Sequence: Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHGNFGNYAVSWFAHWGQGTLVTVSS

SEQ ID NO: 372 - protein, 109 aa, synthetic construct
  REGION 1..109: note = Description of Artificial Sequence: Synthetic polypeptide
QTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 373 - protein, 9 aa, synthetic construct
  REGION 1..9: note = Description of Artificial Sequence: Synthetic peptide
FTFDTYAMN

SEQ ID NO: 374 - protein, 19 aa, synthetic construct
  REGION 1..19: note = Description of Artificial Sequence: Synthetic peptide
RIRSKYNNYATYYADSVKD

SEQ ID NO: 375 - protein, 16 aa, synthetic construct
  REGION 1..16: note = Description of Artificial Sequence: Synthetic peptide
VRHGNFGNYAVSWFAH

SEQ ID NO: 376 - protein, 14 aa, synthetic construct
  REGION 1..14: note = Description of Artificial Sequence: Synthetic peptide
GSSTGAVTTSNYAN

SEQ ID NO: 377 - protein, 7 aa, synthetic construct
  REGION 1..7: note = Description of Artificial Sequence: Synthetic peptide
GTDKRAP

SEQ ID NO: 378 - protein, 9 aa, synthetic construct
  REGION 1..9: note = Description of Artificial Sequence: Synthetic peptide
ALWYSNHWV

SEQ ID NO: 379 - protein, 125 aa, synthetic construct
  REGION 1..125: note = Description of Artificial Sequence: Synthetic polypeptide
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHGSFGNHIVSWFAHWGQGTLVTVSS

SEQ ID NO: 380 - protein, 119 aa, synthetic construct
  REGION 1..119: note = Description of Artificial Sequence: Synthetic polypeptide
QVQLVQSGAEVKKPGSSVKVSCKASGYSFTTYYIHWVRQAPGQGLEWMGWIFPGNDNIKYSEKFKGRVTITADKSTSTAYMELSSLRSEDTAVYYCAIDSVSIYYFDYWGQGTLVTVSS

SEQ ID NO: 381 - protein, 112 aa, synthetic construct
  REGION 1..112: note = Description of Artificial Sequence: Synthetic polypeptide
DIVMTQSPDSLAVSLGERATINCKSSQSLLNSRTRKNYLAWYQQKPGQPPKLLIYWASTRKSGVPDRFSGSGSGTDFTLTISSLQAEDVAVYYCTQSFILRTFGGGTKVEIK

SEQ ID NO: 382 - protein, 10 aa, synthetic construct
  REGION 1..10: note = Description of Artificial Sequence: Synthetic peptide
GYSFTTYYIH

SEQ ID NO: 383 - protein, 17 aa, synthetic construct
  REGION 1..17: note = Description of Artificial Sequence: Synthetic peptide
WIFPGNDNIKYSEKFKG

SEQ ID NO: 384 - protein, 10 aa, synthetic construct
  REGION 1..10: note = Description of Artificial Sequence: Synthetic peptide
DSVSIYYFDY

SEQ ID NO: 385 - protein, 17 aa, synthetic construct
  REGION 1..17: note = Description of Artificial Sequence: Synthetic peptide
KSSQSLLNSRTRKNYLA

SEQ ID NO: 386 - protein, 7 aa, synthetic construct
  REGION 1..7: note = Description of Artificial Sequence: Synthetic peptide
WASTRKS

SEQ ID NO: 387 - protein, 8 aa, synthetic construct
  REGION 1..8: note = Description of Artificial Sequence: Synthetic peptide
TQSFILRT

SEQ ID NO: 388 - protein, 119 aa, synthetic construct
  REGION 1..119: note = Description of Artificial Sequence: Synthetic polypeptide
QVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVSS

SEQ ID NO: 389 - protein, 112 aa, synthetic construct
  REGION 1..112: note = Description of Artificial Sequence: Synthetic polypeptide
DIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGGGTKVEIK

SEQ ID NO: 390 - protein, 10 aa, synthetic construct
  REGION 1..10: note = Description of Artificial Sequence: Synthetic peptide
GFAFTDYYIH

SEQ ID NO: 391 - protein, 17 aa, synthetic construct
  REGION 1..17: note = Description of Artificial Sequence: Synthetic peptide
WISPGNVNTKYNENFKG

SEQ ID NO: 392 - protein, 10 aa, synthetic construct
  REGION 1..10: note = Description of Artificial Sequence: Synthetic peptide
DGYSLYYFDY

SEQ ID NO: 393 - protein, 7 aa, synthetic construct
  REGION 1..7: note = Description of Artificial Sequence: Synthetic peptide
WASTRQS

SEQ ID NO: 394 - protein, 8 aa, synthetic construct
  REGION 1..8: note = Description of Artificial Sequence: Synthetic peptide
TQSHTLRT

SEQ ID NO: 395 - protein, 120 aa, synthetic construct
  REGION 1..120: note = Description of Artificial Sequence: Synthetic polypeptide
MAESGGGSVQTGGSLRLSCAYTASSVCMAWFRQAPGKEREGVAVTREGLTKTGYADSVKGRFAISQDYAKKTLYLQMSSLKPEDTARYYCAARPTSPCTVDGELLASTYNYWGQGTQVTV

SEQ ID NO: 396 - protein, 120 aa, synthetic construct
  REGION 1..120: note = Description of Artificial Sequence: Synthetic polypeptide
MAESGGGSVQTGGSLRLSCAYTASSVCMAWFRQAPGKEREGVAVTREGLTKTGYADSVKGRFAISQDYAKKTLYLQMSSLKPEDTARYYCAARPTSPCTVDGELLASTYDYWGQGTQVTV

SEQ ID NO: 397 - protein, 120 aa, synthetic construct
  REGION 1..120: note = Description of Artificial Sequence: Synthetic polypeptide
MAESGGGSVQTGGSLRLSCAYTASSVCMAWFRQAPGKEREGVAVTREGLTQTGYADSVKGRFAISQDYAKKTLYLQMSSLKPEDTARYYCAARPTSPCTVDGELLASTYNYWGQGTQVTV

SEQ ID NO: 398 - protein, 120 aa, synthetic construct
  REGION 1..120: note = Description of Artificial Sequence: Synthetic polypeptide
MAESGGGSVQTGGSLRLSCAYTASSVCMAWFRQAPGKEREGVAVTREGLTQTGYADSVKGRFAISQDYAKKTLYLQMSSLKPEDTARYYCAARPTSPCTVDGELLASTYDYWGQGTQVTV

SEQ ID NO: 399 - protein, 16 aa, synthetic construct
  REGION 1..16: note = Description of Artificial Sequence: Synthetic peptide
VRHGSFGNHIVSWFAH

SEQ ID NO: 400 - protein, 40 aa, synthetic construct
  REGION 1..40: note = Description of Artificial Sequence: Synthetic polypeptide
  REGION 1..40: note = MISC_FEATURE - This sequence may encompass 1-20 "Gly Ser" repeating units
GSGSGSGSGSGSGSGSGSGSGSGSGSGSGSGSGSGSGSGS

SEQ ID NO: 401 - protein, 60 aa, synthetic construct
  REGION 1..60: note = Description of Artificial Sequence: Synthetic polypeptide
  REGION 1..60: note = MISC_FEATURE - This sequence may encompass 1-20 "Gly Gly Ser" repeating units
GGSGGSGGSGGSGGSGGSGGSGGSGGSGGSGGSGGSGGSGGSGGSGGSGGSGGSGGSGGS

SEQ ID NO: 402 - protein, 80 aa, synthetic construct
  REGION 1..80: note = Description of Artificial Sequence: Synthetic polypeptide
  REGION 1..80: note = MISC_FEATURE - This sequence may encompass 1-20 "Gly Gly Gly Ser" repeating units
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 403 - protein, 80 aa, synthetic construct
  REGION 1..80: note = Description of Artificial Sequence: Synthetic polypeptide
  REGION 1..80: note = MISC_FEATURE - This sequence may encompass 1-20 "Gly Gly Ser Gly" repeating units
GGSGGGSGGGSGGGSGGGSGGGSGGGSGGGSGGGSGGGSGGGSGGGSGGGSGGGSGGGSGGGSGGGSGGGSGGGSGGGSG

SEQ ID NO: 404 - protein, 100 aa, synthetic construct; identical to SEQ ID NO: 301
  REGION 1..100: note = Description of Artificial Sequence: Synthetic polypeptide
  REGION 1..100: note = MISC_FEATURE - This sequence may encompass 1-20 "Gly Gly Ser Gly Gly" repeating units

SEQ ID NO: 405 - protein, 100 aa, synthetic construct; identical to SEQ ID NO: 300
  REGION 1..100: note = Description of Artificial Sequence: Synthetic polypeptide
  REGION 1..100: note = MISC_FEATURE - This sequence may encompass 1-20 "Gly Gly Gly Gly Ser" repeating units

SEQ ID NO: 406 - protein, 634 aa, synthetic construct
  REGION 1..634: note = Description of Artificial Sequence: Synthetic polypeptide
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSSGGGGSGGGGSGGGGSEIVLTQSPATLSLSPGERATLSCSASSSVSYMHWYQQKPGQAPRLLIYDTSKLASGIPARFSGSGSGTDFTLTISSLEPEDFAVYYCFQGSVYPFTFGQGTKLEIKRHHHHHHHHHH

SEQ ID NO: 407 - protein, 10 aa, synthetic construct
  REGION 1..10: note = Description of Artificial Sequence: Synthetic 10xHis tag
HHHHHHHHHH